(19) United States
(12) Patent Application Publication
(10) Pub. No.: US 2015/0018239 A1
(43) Pub. Date: Jan. 15, 2015

(54) BIOMARKER SET FOR IDENTIFYING A SEVERE FORM OF CANCER

(71) Applicant: Deutsches Krebsforschungszentrum, Heidelberg (DE)

(72) Inventors: Johanna Sonntag, Neuenstadt (DE); Christian Bender, Mainz (DE); Ulrike Korf, Heidelberg (DE); Stefan Wiemann, Sandhausen (DE)

(73) Assignee: Deutsches Krebsforschungszentrum, Heidelberg (DE)

(21) Appl. No.: 14/377,374

(22) PCT Filed: Feb. 8, 2013
(86) PCT No.: PCT/EP2013/052541

§ 371 Date: Aug. 7, 2014

(30) Foreign Application Priority Data
Feb. 10, 2012 (EP) 12154916.6

(51) Int. Cl. G01N 33/574

(52) U.S. Cl. CPC G01N 33/57415; G01N 33/57488; G01N 2800/52; G01N 2800/56
USPC 506/9; 506/18; 435/7.1; 435/7.4; 435/7.23

(57) ABSTRACT

The present invention relates to a method for differentiating between i) a severe form of cancer and ii) a mild form of cancer, comprising a) determining the amounts of gene product of at least the genes coding for ribosomal protein S6 (RPS6), nucleoside diphosphate kinase (NME/NDKA), and caveolin-1, in a sample from a subject, b) comparing the amounts obtained in step a) to reference amounts, and c) differentiating between a severe form of cancer and a mild form of cancer, wherein an increased amount of products of the genes coding for RPS6 and NME/NDKA and a decreased amount of product of the gene coding for caveolin-1 are indicative of a severe form of cancer. The invention further relates to the use of antibodies specifically recognizing a polypeptide selected from the list consisting of RPS6, NME/NDKA, and caveolin-1, for differentiating between a severe form of cancer and a mild form of cancer. Furthermore, the invention relates to a detection agent specifically recognizing a polypeptide selected from the list consisting of RPS6, NME/NDKA, and caveolin-1, for use in diagnosing, a device and a kit for differentiating between a severe form of cancer and a mild form of cancer.

The present invention relates to a method for differentiating between i) a severe form of cancer and ii) a mild form of cancer, comprising a) determining the amounts of gene product of at least the genes coding for ribosomal protein S6 (RPS6), nucleoside diphosphate kinase (NME/NDKA), and caveolin-1, in a sample from a subject, b) comparing the amounts obtained in step a) to reference amounts, and c) differentiating between a severe form of cancer and a mild form of cancer, wherein an increased amount of products of the genes coding for RPS6 and NME/NDKA and a decreased amount of product of the gene coding for caveolin-1 are indicative of a severe form of cancer. The invention further relates to the use of antibodies specifically recognizing a polypeptide selected from the list consisting of RPS6, NME/NDKA, and caveolin-1, for differentiating between a severe form of cancer and a mild form of cancer. Furthermore, the invention relates to a detection agent specifically recognizing a polypeptide selected from the list consisting of RPS6, NME/NDKA, and caveolin-1, for use in diagnosing, a device and a kit for differentiating between a severe form of cancer and a mild form of cancer.

Cancer has been recognized as a heterogeneous disease that consists of different intrinsic molecular subtypes. Typically, not all molecular subtypes react in the same or in a similar way to a specific treatment applied to a patient. Thus, treatment—frequently afflicted with severe side effects—often is applied to patients whose tumors are not in a molecular state to be affected by said treatment, which makes treatment futile at best. It is therefore of high importance to find means and methods allowing a prediction on possible therapy outcome to be made before therapy is started.

One such heterogenous form of cancer is breast cancer, of which hormone receptor positive breast cancer or luminal breast cancer presents the largest group with 70-80% of newly diagnosed breast cancer patients (Perou C M, Sorlie T, Eisen M B, van de Rijn M, Jeffrey S S, Rees C A, et al. Molecular portraits of human breast tumours. Nature. 2000 Aug. 17; 406(6797):747-52. Sorlie T, Tibshirani R, Parker J, Hastie T, Marron J S, Nobel A, et al. Repeated observation of breast tumor subtypes in independent gene expression data sets. Proc Natl Acad Sci USA. 2003 Jul. 8; 100(14):8418-23.). Current guidelines for a molecular classification of breast cancer are based on a quantification of estrogen receptor α (ESR1) and progesteron receptor (PR), HER2, and the cell cycle progression marker Ki-67 by immunohistochemistry to approximate the intrinsic molecular suptypes (Goldhirsch A, Wood W C, Coates A S, Gelber R D, Thurlimann B, Senn H J. Strategies for subtypes—dealing with the diversity of breast cancer: highlights of the St. Gallen International Expert Consensus on the Primary Therapy of Early Breast Cancer 2011. Ann Oncol. August; 22(8):1736-47. Cheang M C, et al., loc cit). In case histologic grading is not available to distinguish luminal A breast cancer from the clinically more aggressive subtype luminal B, other clinicopathological paramenters such as Ki-67 quantification can be used for the distinction between luminal A and luminal B (Goldhirsch A, Wood W C, Coates A S, Gelber R D, Thurlimann B, Senn H J. Strategies for subtypes—dealing with the diversity of breast cancer: highlights of the St. Gallen International Expert Consensus on the Primary Therapy of Early Breast Cancer 2011. Ann Oncol. August; 22(8):1736-47. Cheang M C, et al., loc cit).

The histologic grade is determined by semi-quantitative methods describing morphologic features related to the differentiation state of tumor specimen ranging from well differentiated “grade 1” tumors to poorly differentiated “grade 3” tumors. Tumors with intermediate differentiation constitute the class of “grade 2” tumors (Elston, C. W., and Ellis, I. O. (1991), Pathological prognostic factors in breast cancer. I. The value of histological grade in breast cancer: experience from a large study with long-term follow-up. Histopathology, 19(5):403-10.).

Breast tumors characterized as luminal “grade 3” tumor stain strongly for Ki-67 and generally respond well to chemotherapeutic treatments. Chemotherapies were less successful in patients with luminal “grade 1” tumors (Fasching P A, Heusinger K, Haeberle L, Niklos M, Hein A, Bayer C M, et al. Ki67, chemotherapy response, and prognosis in breast cancer patients receiving neoadjuvant treatment. BMC Cancer. 11:486.), however, due to the slow progression of luminal grade 1 tumors, patients typically can be treated adequately with anti-estrogens alone. Treatment decisions are most difficult for the majority of patients with luminal breast cancer since their tumors were classified as “grade 2”. Luminal “grade 2” tumors constitute a highly heterogeneous class as demonstrated by expression profiling and resulted in the introduction of the genomic grade index (Sotiriou C, Wirapati P, Loi S, Harris A, Fox S, Smeds J, et al. Gene expression profiling in breast cancer: understanding the molecular basis of histologic grade to improve prognosis. J Natl Cancer Inst. 2006 Feb. 15; 98(4):262-72.). This 97 gene signature separates “grade 2” tumors into “grade 1”-like (low risk) and “grade 3”-like (high risk) tumors and hence provides information for treatment decisions (Filho O M, Ignatiadis M, Sotiriou C. Genomic Grade Index: An important tool for assessing breast cancer tumor grade and prognosis. Crit Rev Oncol Hematol. January; 77(1):20-9.). However, the determination of intrinsic gene signatures by genetic testing requires the preparation of mRNA from tumors or biopsy samples and thus present working steps that consume additional tumor material, additional analysis time, and are costly. In conclusion, extending the panel of molecular features that can be determined by routine immunohistochemistry would present a faster and more cost-effective approach to identify those breast cancer patients that truly benefit from chemotherapeutic treatment regimens.

Accordingly, the technical problem underlying the present invention can be seen as the provision of means and methods for complying with the aforementioned needs. The technical problem is solved by the embodiments characterized in the claims and herein below.

Therefore, the present invention relates to a method for differentiating between i) a severe form of cancer and ii) a mild form of cancer, comprising a) determining the amounts of gene product of at least the genes coding for ribosomal protein S6 (RPS6), nucleoside diphosphate kinase (NME/NDKA), and caveolin-1, in a sample from a subject, b) comparing the amounts obtained in step a) to reference amounts, and c) differentiating between a severe form of cancer and a mild form of cancer, wherein an increased amount of products of the genes coding for RPS6 and NME/NDKA and a decreased amount of product of the gene coding for caveolin-1 are indicative of a severe form of cancer.

The method of the present invention, preferably, is an in vitro method. Moreover, it may comprise steps in addition to those explicitly mentioned above. For example, further steps may relate, e.g., to pre-treatment of the sample of step a) or evaluation of the results obtained by the method. Additionally, internal controls, such as sample quality controls or performance controls may be used. The method may be carried out manually or assisted by automation. Preferably, steps (a) to (c) may in total or in part be assisted by automation, e.g. by suitable robotic equipment for determining the amounts of gene products in step (a).

The term “differentiating”, as used herein, means to distinguish between a severe form of cancer and a mild form of cancer in a subject. As will be understood by those skilled in the art, the aforementioned differentiation is usually not intended to be correct for 100% of the subjects to be analyzed. The term, however, requires that the differentiation will be valid for a statistically significant portion of the subjects to be analyzed. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the differentiation will be correct for at least 60%, at least 70%, at least 80%, or at least 90% of the subjects of a given cohort or population.

The term “cancer”, as used in this specification, relates to a solid malignant neoplasm. Preferably, the cancer is breast cancer. More preferably, the cancer is hormone-receptor positive breast cancer, most preferably with intermediate grading. The skilled person understands the term hormone-receptor positive breast cancer, relating to a subclass of breast cancer expressing estrogen-receptor α (ERα). Grading of hormone-receptor positive breast cancer relates to histologic grade determination by semi-quantitative methods describing morphologic features related to the differentiation state of a tumor sample and ranging from well differentiated “grade 1” tumors to poorly differentiated “grade 3” tumors. The term “intermediate grading” thus relates to tumors with intermediate differentiation, graded according to the criteria specified above as “grade 2” tumors.

The term “mild form of cancer”, preferably, relates to a form of cancer progressing slowly. Preferably, the term relates to a form of cancer progressing and responding to therapy to a similar extent as a grade 1 tumor does. More preferably, a mild form of cancer is a form of cancer with a high probability to respond to therapy, e.g., preferably, anti-estrogen therapy or chemotherapy. Most preferably, a mild form of cancer is a form of cancer not requiring chemotherapy, e.g. a hormone-receptor positive breast cancer being manageable by anti-estrogen therapy alone or without anti-tumor treatment. Preferably, the term “cancer responding to chemotherapy” relates to a cancer not progressing under chemotherapy, more preferably, a cancer responding to chemotherapy is a cancer regressing under chemotherapy, and most preferably, the cancer responding to chemotherapy is a cancer completely regressing and not relapsing within five years after chemotherapy.

As used herein, the term “severe form of cancer”, preferably, relates to a form of cancer progressing fast and, preferably, responding to therapy, preferably chemotherapy and/or targeted therapy, to a similar extent as a grade 3 tumor does. More preferably, a severe form of cancer is a form of cancer requiring chemotherapy, e.g. a hormone-receptor positive breast cancer requiring chemotherapy and/or targeted therapy, most preferably with a low probability to respond to chemotherapy. Preferably, the term “cancer not responding to chemotherapy” relates to a cancer resulting in a relapse within five years after chemotherapy, more preferably, the cancer not responding to chemotherapy is a cancer not completely regressing under chemotherapy. Most preferably, the cancer not responding to chemotherapy is a cancer progressing under chemotherapy. In a preferred embodiment, the term “severe form of hormone receptor positive breast cancer not responding to chemotherapy” relates to a cancer not completely regressing under hormone therapy, more preferably, the term relates to a cancer resulting in a relapse within five years after anti-estrogen therapy. Most preferably, the term relates to a cancer progressing under anti-hormone therapy and therefore requiring chemotherapy.

The term “chemotherapy” is understood by the skilled person, relating to cancer treatment with an antineoplastic drug or a combination of such drugs. It is clear to the skilled person that chemotherapy according to the present invention may be accompanied by other forms of therapy, e.g. surgical removal of the tumor.

The term “anti-estrogen” therapy relates to the treatment of breast cancer and other diseases by administering to a subject at least one aromatase-inhibitor inhibiting aromatase (EC 1.14.14.1), the enzyme responsible for the aromatization of androgens into estrogens. The anti-estrogen is selected from the groups of steroidal aromatase inhibitors, like, e.g. exemestane, and non-steroidal aromatase inhibitors, like, e.g. anastrozole. Also included as anti-estrogen therapy is a treatment comprising administering to a subject a selective estrogen receptor modulator, e.g. tamoxifen, raloxifene, lasofoxifene or toremifene.

As used herein, the term “gene product” relates to a, preferably macromolecular, physical entity, the presence of which in a cell depends on the expression of said gene in said cell. The mechanisms of gene expression are well-known to the one skilled in the art to include the basic mechanisms of transcription, i.e. formation of RNA corresponding to the said gene or parts thereof, and translation, i.e. production of polypeptide molecules having an amino acid sequence encoded by said RNA according to the genetic code; it is well-known to the one skilled in the art that other cellular processes may be involved in gene expression as well, e.g. RNA processing, RNA editing, proteolytic processing, protein editing, and the like. The term gene product thus includes RNA, preferably mRNA, as well as polypeptides expressed from said gene. It is clear from the above that the term gene product also includes fragments of said RNA(s), preferably with a length of at least ten, at least twelve, at least 20, at least 50, or at least 100 nucleotides, and fragments (peptides) from said polypeptides, preferably with a length of at least eight, at least ten, at least twelve, at least 15, at least 20 amino acids.

“Determining” the amount of a gene product relates to measuring the amount of said gene product, preferably semi-quantitatively or quantitatively. Measuring can be done directly or indirectly. Preferably, measuring is performed on a processed sample, said processing comprising extraction of polynucleotides or polypeptides from the sample. Also preferably, the amount of gene product is determined on a tissue section from said sample. The amount of the polynucleotides of the present invention can be determined with several methods well-known in the art. Quantification preferably is absolute, i.e. relating to a specific number of polynucleotides or, more preferably, relative, i.e. measured in arbitrary normalized units. Preferably, a normalization is carried out by calculating the ratio of a number of specific polynucleotides and total number of polynucleotides or a reference amplification product comprised by a sample as set forth elsewhere herein in detail. Methods allowing for absolute or relative quantification are well known in the art. E.g., quantitative PCR methods are methods for relative quantification; if a calibration curve is incorporated in such an assay, the relative quantification can be used to obtain an absolute quantification. Other methods known are, e.g. nucleic acid sequence-based amplification (NASBA) or the Branched DNA Signal Amplification Assay method in combination with dot blot or luminex detection of amplified polynucleotides. Preferably, the polynucleotide amounts are normalized polynucleotide amounts, i.e. the polynucleotide amounts obtained are set into relation to at least one reference amplification product, thereby, preferably, setting the polynucleotide amounts into relation to the number of cells in the sample and/or the efficiency of polynucleotide amplification. Thus, preferably, the reference amplification product is a product obtained from a polynucleotide known to have a constant abundancy in each cell, i.e. a polynucleotide comprised in most, preferably all, cells of a sample in approximately the same amount. More preferably, the reference amplification product is amplified from a chromosomal or mitochondrial gene or from the mRNA of a housekeeping gene.

The amount of peptides or polypeptides of the present invention can be determined in various ways. Direct measuring relates to measuring the amount of the peptide or polypeptide based on a signal which is obtained from the peptide or polypeptide itself and the intensity of which directly correlates with the number of molecules of the peptide present in the sample. Such a signal—sometimes referred to herein as intensity signal—may be obtained, e.g., by measuring an intensity value of a specific physical or chemical property of the peptide or polypeptide. Indirect measuring includes measuring of a signal obtained from a secondary component (i.e. a component not being the peptide or polypeptide itself) or a biological read out system, e.g., measurable cellular responses, ligands, labels, or enzymatic reaction products.

In accordance with the present invention, determining the amount of a peptide or polypeptide can be achieved by all known means for determining the amount of a peptide in a sample. Said means comprise immunoassay and/or immunohistochemistry devices and methods which may utilize labeled molecules in various sandwich, competition, or other assay formats. Said assays will develop a signal which is indicative for the presence or absence of the peptide or polypeptide. Moreover, the signal strength can, preferably, be correlated directly or indirectly (e.g. reverse-proportional) to the amount of polypeptide present in a sample. Further suitable methods comprise measuring a physical or chemical property specific for the peptide or polypeptide such as its precise molecular mass or NMR spectrum. Said methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays, Cobalt Binding Assays, and latex agglutination assays.

Also preferably, determining the amount of a peptide or polypeptide comprises the step of measuring a specific intensity signal obtainable from the peptide or polypeptide in the sample. As described above, such a signal may be the signal intensity observed at an m/z variable specific for the peptide or polypeptide observed in mass spectra or a NMR spectrum specific for the peptide or polypeptide.

Determining the amount of a peptide or polypeptide may, preferably, comprise the steps of (a) contacting the peptide with a specific ligand, (b) (optionally) removing non-bound ligand, (c) measuring the amount of bound ligand. The bound ligand will generate an intensity signal. Binding according to the present invention includes both covalent and non-covalent binding. A ligand according to the present invention can be any compound, e.g., a peptide, polypeptide, nucleic acid, or small molecule, binding to the peptide or polypeptide described herein. Preferred ligands include antibodies, nucleic acids, peptides or polypeptides such as receptors or binding partners for the peptide or polypeptide and fragments thereof comprising the binding domains for the peptides, and aptamers, e.g. nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g. phage display. Antibodies as referred to herein include both polyclonal and monoclonal antibodies, as well as fragments thereof, such as Fv, Fab and F(ab)2 fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the peptide or polypeptide. Specific binding according to the present invention means that the ligand or agent should not bind substantially to (“cross-react” with) another peptide, polypeptide or substance present in the sample to be analyzed. Preferably, the specifically bound peptide or polypeptide should be bound with at least 3 times higher, more preferably at least 10 times higher and even more preferably at least 50 times higher affinity than any other relevant peptide or polypeptide. Nonspecific binding may be tolerable, if it can still be distinguished and measured unequivocally, e.g. according to its size on a Western Blot, or by its relatively higher abundance in the sample. Binding of the ligand can be measured by any method known in the art. Preferably, said method is semi-quantitative or quantitative. Suitable methods are described in the following.

First, binding of a ligand may be measured directly, e.g. by NMR or surface plasmon resonance. Second, if the ligand also serves as a substrate of an enzymatic activity of the peptide or polypeptide of interest, an enzymatic reaction product may be measured (e.g. the amount of a protease can be measured by measuring the amount of cleaved substrate, e.g. on a Western Blot). Alternatively, the ligand may exhibit enzymatic properties itself and the “ligand/peptide or polypeptide” complex or the ligand which was bound by the peptide or polypeptide, respectively, may be contacted with a suitable substrate allowing detection by the generation of an intensity signal. For measurement of enzymatic reaction products, preferably the amount of substrate is saturating. The substrate may also be labeled with a detectable label prior to the reaction. Preferably, the sample is contacted with the substrate for an adequate period of time. An adequate period of time refers to the time necessary for a detectable, preferably measurable, amount of product to be produced. Instead of measuring the amount of product, the time necessary for appearance of a given (e.g. detectable) amount of product can be measured. Third, the ligand may be coupled covalently or non-covalently to a label allowing detection and measurement of the ligand. Labelling may be done by direct or indirect methods. Direct labelling involves coupling of the label directly (covalently or non-covalently) to the ligand. Indirect labelling involves binding (covalently or non-covalently) of a secondary ligand to the first ligand. The secondary ligand should specifically bind to the first ligand. Said secondary ligand may be coupled with a suitable label and/or be the target (receptor) of tertiary ligand binding to the secondary ligand. The use of secondary, tertiary or even higher order ligands is often used to increase the signal intensity. Suitable secondary and higher order ligands may include antibodies, secondary antibodies, and the well-known streptavidin-biotin system (Vector Laboratories, Inc.). The ligand or substrate may also be “tagged” with one or more tags as known in the art. Such tags may then be targets for higher order ligands. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate), CDP-Star™ (Amersham Biosciences), ECF™ (Amersham Biosciences). A suitable enzyme-substrate combination may result in a colored reaction product, fluorescence or chemo luminescence, which can be measured according to methods known in the art (e.g. using a light-sensitive film or a suitable camera system). As for measuring the enzymatic reaction, the criteria given above apply analogously. Typical fluorescent labels include fluorescent proteins (such as GFP and its derivatives), Cy3, Cy5, Texas Red, Fluorescein, and the Alexa dyes (e.g. Alexa 568). Further fluorescent labels are available e.g. from Molecular Probes (Oregon). Also the use of quantum dots as fluorescent labels is contemplated. Typical radioactive labels include 35S, I25I, 32P, 33P and the like. A radioactive label can be detected by any method known and appropriate, e.g. a light-sensitive film or a phosphor imager. Suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests, like e.g. reverse phase protein arrays or antibody arrays. Further methods known in the art (such as gel electrophoresis, 2D gel electrophoresis, SDS polyacrylamid gel electrophoresis (SDS-PAGE), Western Blotting, and mass spectrometry), can be used alone or in combination with labelling or other detection methods as described above.

The amount of a peptide or polypeptide may be, also preferably, determined as follows: (a) contacting a solid support comprising a ligand for the peptide or polypeptide as specified above with a sample comprising the peptide or polypeptide and (b) measuring the amount peptide or polypeptide which is bound to the support. The ligand, preferably chosen from the group consisting of nucleic acids, peptides, polypeptides, antibodies and aptamers, is preferably present on a solid support in immobilized form. Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Suitable methods for fixing/immobilizing said ligand are well known and include, but are not limited to ionic, hydrophobic, covalent interactions and the like. It is also contemplated to use “suspension arrays” as arrays according to the present invention (Nolan 2002, Trends Biotechnol. 20(1):9-12). In such suspension arrays, the carrier, e.g. a microbead or microsphere, is present in suspension. The array consists of different microbeads or microspheres, possibly labeled, carrying different ligands. Methods of producing such arrays, for example based on solid-phase chemistry and photo-labile protective groups, are generally known (U.S. Pat. No. 5,744,305).

The method of the present invention comprises determining the amounts of gene product of at least the genes coding for ribosomal protein S6 (RPS6), nucleoside diphosphate kinase (NME/NDKA), and caveolin-1. Preferably, the method of the present invention further comprises determining the amount of gene product of the gene coding for KI-67 and/or the amount of gene product of the gene coding for DNA topoisomerase 2-alpha (TOP2A). Said genes and their preferred products are known to the skilled person and the respective sequences have been deposited in databases; relevant accession numbers and SEQ ID NOs are compiled in Table 1. It is understood by the skilled person that the gene products are referenced as biomarkers, not as specific polynucleotides or polypeptides. Accordingly, the aforementioned polynucleotides and polypeptides having the specific sequences deposited under the Genbank accession numbers are to be understood as exemplary sequences representing a biomarker. Encompassed as gene products according to the present invention are also variant polynucleotides which vary due to at least one nucleotide addition, substitution and/or deletion form the polynucleotide having the specific sequence as long as they are also suitable as biomarkers for expression of one of the genes as discussed above. Preferably, the variant polynucleotides are at least 70%, at least 75%, at least 80%, at least 85%, at least 90%, at least 95%, at least 98% or at least 99% identical to the specific polynucleotides. The term “identical” as used herein refers to sequence identity characterized by determining the number of identical nucleotides between two nucleic acid sequences or amino acid sequences wherein the sequences are aligned so that the highest order match is obtained. It can be calculated using published techniques or methods codified in computer programs such as, for example, BLASTP, BLASTN or FASTA (Altschul 1990, J Mol Biol 215, 403). The percent identity values are, in one aspect, calculated over the entire nucleic acid or amino acid sequence. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (Higgins 1989, CABIOS 5, 151) or the programs Gap and BestFit (Needleman 1970, J Mol Biol 48; 443; Smith 1981, Adv Appl Math 2, 482), which are part of the GCG software packet (Genetics Computer Group 1991, 575 Science Drive, Madison, Wis., USA 53711), may be used. The sequence identity values recited above in percent (%) are to be determined, in another aspect of the invention, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments. If a variant polynucleotide is suitable as a biomarker for expression of one of the genes can be assessed by determining according to the methods specified herein if the variant polynucleotide has essentially the same expression pattern as the biomarker it is a variant of. Also encompassed according to the present invention are variant polypeptides which vary due to at least one amino acid addition, substitution and/or deletion form the polypeptide having the specific sequence as long as they are also suitable as biomarkers for expression of one of the genes as discussed above. Preferably, the variant polypeptides are at least 70%, at least 75%, at least 80%, at least 85%, at least 90%, at least 95%, at least 98% or at least 99% identical to the specific polypeptides. The term “identical” as used herein refers to sequence identity characterized by determining the number of identical amino acids between two nucleic acid sequences or amino acid sequences according to the methods specified herein above. It also understood by the skilled person that the method of the present invention may comprise determining the amounts of further gene products, e.g. non-limiting, products of the genes coding for HER2, estrogen receptor α, or progesterone receptor.

TABLE 1
Accession numbers and SEQ ID NOs of the polypeptides/biomarkers of the present specification

| | | |
|---|---|---|
| gene | mRNA/cDNA | protein |

| polypeptide/ | | SEQ ID | | SEQ ID | | SEQ |
|---|---|---|---|---|---|---|
| biomarker name | Genbank Acc No | NO | Genbank Acc No | NO | Genbank Acc No | ID NO |
| ribosomal protein | NC_000009.11 | 1 | NM_001010.2 | 2 | NP_001001.2 | 3 |
| S6 (RPS6) | GI: 224589821 | | GI: 17158043 | | GI: 17158044 | |
| nucleoside | NC_000017.10 | 4 | NM_198175.1 | 5 | NP_937818.1 | 6 |
| diphosphate | GI: 224589808 | | GI: 38045912 | | GI: 38045913 | |
| kinase | | | | | | |
| (NME/NDKA) | | | | | | |
| caveolin-1 | NC_000007.13 | 7 | NM_001753.4 | 8 | NP_001744.2 | 9 |
| | GI: 224589819 | | GI: 290542357 | | GI: 15451856 | |
| KI-67 antigen | NC_000010.10 | 10 | NM_002417.4 | 11 | NP_002408.3 | 12 |
| (KI67) | GI: 224589801 | | GI: 225543213 | | GI: 103472005 | |
| DNA | NC_000017.10 | 13 | NM_001067.3 | 14 | NP_001058.2 | 15 |
| topoisomerase | GI: 224589808 | | GI: 300193028 | | GI: 19913406 | |
| 2-alpha (TOP2A) | | | | | | |

The term “subject”, as used herein, relates to a mammal and, preferably, to a human. The subject, preferably, suffers from cancer. More preferably, the subject is a female or a male suffering from breast cancer.

The term “sample” refers to a sample from a tissue or an organ or to a sample of wash/rinse fluid obtained from an outer or inner body surface, preferably comprising at least 70%, at least 80%, or at least 90% cancer cells. Samples can be obtained by use of brushes, (cotton) swabs, spatula, rinse/wash fluids, punch biopsy devices, puncture of cavities with needles or surgical instrumentation. However, samples obtained by well known techniques including, preferably, biopsies from the urogenital tract, perianal regions, anal canal, the oral cavity, the upper aerodigestive tract are also included as samples of the present invention. More preferably, samples are tumor tissue or biopsy material from a solid tumor.

“Comparing” as used herein encompasses comparing the amount of the gene products referred to herein which are comprised by the sample to be analyzed with an amount of the said gene products in a suitable reference sample as specified elsewhere herein in this description. It is to be understood that comparing as used herein refers to a comparison of corresponding parameters or values, e.g., an absolute amount of the gene products as referred to herein is compared to an absolute reference amount of said gene products; a concentration of the gene products as referred to herein is compared to a reference concentration of said gene products; or an intensity signal obtained from the gene products as referred to herein in a test sample is compared to the same type of intensity signal of said gene products in a reference sample. The comparison referred to in the methods of the present invention may be carried out manually or computer assisted. For a computer assisted comparison, the value of the determined amount or ratio may be compared to values corresponding to suitable references which are stored in a database by a computer program. The computer program may further evaluate the result of the comparison by means of an expert system. Accordingly, the result of the identification referred to herein may be automatically provided in a suitable output format.

The term “reference amount” as used herein refers to an amount of gene products, which allows assessing if a mild form of cancer or a severe form of cancer is to be assumed for the subject from which the sample is derived. A suitable reference value may be determined from a reference sample to be analyzed together, i.e. simultaneously or subsequently, with the sample. It is clear for the skilled person that the reference value for one gene product of the present invention preferably is different from the reference value of a second gene product, i.e. preferably, each gene product has an independent reference value.

Reference amounts can, in principle, be calculated for a group or cohort of subjects as specified herein based on the average or mean values for a given gene product by applying standard methods of statistics. In particular, accuracy of a test such as a method aiming to diagnose an event, or not, is best described by its receiver-operating characteristics (ROC) (see especially Zweig 1993, Clin. Chem. 39:561-577). The ROC graph is a plot of all of the sensitivity versus specificity pairs resulting from continuously varying the decision threshold over the entire range of data observed. The clinical performance of a diagnostic method depends on its accuracy, i.e. its ability to correctly allocate subjects to a certain prognosis or diagnosis. The ROC plot indicates the overlap between the two distributions by plotting the sensitivity versus 1-specificity for the complete range of thresholds suitable for making a distinction. On the y-axis is sensitivity, or the true-positive fraction, which is defined as the ratio of number of true-positive test results to the product of number of true-positive and number of false-negative test results. This has also been referred to as positivity in the presence of a disease or condition. It is calculated solely from the affected subgroup. On the x-axis is the false-positive fraction, or 1-specificity, which is defined as the ratio of number of false-positive results to the product of number of true-negative and number of false-positive results. It is an index of specificity and is calculated entirely from the unaffected subgroup. Because the true- and false-positive fractions are calculated entirely separately, by using the test results from two different subgroups, the ROC plot is independent of the prevalence of the event in the cohort. Each point on the ROC plot represents a sensitivity/-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the overall accuracy of the test. Dependent on a desired confidence interval, a threshold can be derived from the ROC curve allowing for the diagnosis or prediction for a given event with a proper balance of sensitivity and specificity, respectively. Accordingly, the reference to be used for the methods of the present invention can be generated, preferably, by establishing a ROC for said cohort as described above and deriving a threshold amount there from. Dependent on a desired sensitivity and specificity for a diagnostic method, the ROC plot allows deriving suitable thresholds.

Preferably, the reference amount as used herein is derived from samples from a mild form of cancer and of a severe form of cancer as specified herein above. E.g. samples from grade 1 tumors are suitable for deriving reference amounts for a mild form of cancer, and/or samples from grade 3 tumors are suitable for deriving reference amounts for a severe form of cancer. Also preferably, the reference amount is derived from samples of subjects obtained before treatment, but for which it is known if their donors required or responded to chemotherapy treatment or not. This reference amount level may be a discrete figure or may be a range of figures. Evidently, the reference level or amount may vary between individual species of gene products. The reference amount applicable for an individual subject may vary depending on various physiological parameters such as age, gender, or subpopulation. Thus, a suitable reference amount may be determined by the methods of the present invention from a reference sample to be analyzed together, i.e. simultaneously or subsequently, with the test sample. Moreover, a threshold amount can be preferably used as a reference amount. Preferably, an amount of gene products which is above the threshold amount is indicative of a mild form of cancer; and an amount of gene products which is equal or below the threshold amount will be indicative for a severe form of cancer. It is to be understood that the aforementioned amounts may vary due to statistics and errors of measurement.

It has been found that, preferably, an increased amount of products of the genes coding for RPS6 and NME/NDKA and a decreased amount of product of the gene coding for caveolin-1 are indicative of a severe form of cancer. Thus, a decreased amount of products of the genes coding for RPS6 and NME/NDKA and an increased amount of product of the gene coding for caveolin-1 are indicative of a mild form of cancer. It has been further found that an increased amount of product of the gene coding for KI-67 and/or of the gene coding for TOP2A is further indicative of a severe form of cancer, and thus, that a decreased amount of product of the gene coding for KI-67 and/or of the gene coding for TOP2A is further indicative of a mild form of cancer.

The definitions made above apply mutatis mutandis to the following:

In a further preferred embodiment, the present invention relates to the use of at least three antibodies, each of said antibodies specifically recognizing a different polypeptide selected from the list consisting of RPS6, NME/NDKA, and caveolin-1, for differentiating between i) a severe form of cancer and ii) a mild form of cancer.

Antibodies against the polypeptides of the invention can be prepared by well known methods using a purified polypeptide according to the invention or a suitable fragment derived therefrom as an antigen. A fragment which is suitable as an antigen may be identified by antigenicity determining algorithms well known in the art. Such fragments may be obtained either from the polypeptide of the invention by proteolytic digestion or may be a synthetic peptide. Preferably, the antibody of the present invention is a monoclonal antibody, a polyclonal antibody, a single chain antibody, a human or humanized antibody or primatized, chimerized or fragment thereof. Also comprised as antibodies by the present invention are a bispecific antibody, a synthetic antibody, an antibody fragment, such as Fab, Fv or scFv fragments etc., or a chemically modified derivative of any of these. The antibody of the present invention shall specifically bind (i.e. does not cross react with other polypeptides or peptides) to the polypeptide of the invention. Specific binding can be tested by various well known techniques. Antibodies or fragments thereof can be obtained by using methods which are described, e.g., in Harlow and Lane “Antibodies, A Laboratory Manual”, CSH Press, Cold Spring Harbor, 1988. Monoclonal antibodies can be prepared by the techniques originally described in Köhler and Milstein, Nature 256 (1975), 495, and Galfré, Meth. Enzymol. 73 (1981), 3, which comprise the fusion of mouse myeloma cells to spleen cells derived from immunized mammals.

In a further preferred embodiment, the present invention relates to a detection reagent specifically recognizing a polypeptide selected from the list consisting of RPS6, NME/NDKA, and caveolin-1, for use in diagnosing i) a severe form of cancer or ii) a mild form of cancer, comprising a) applying said detection agent to said subject, b) determining the amount of RPS6, NME/NDKA, and caveolin-1 in a tissue suspected to comprise cancer cells, c) comparing the amount determined in step b) to a reference amount determined from tissue not suspected to comprise cancer cells, d) determining a difference between the amount determined in step a) and the amount determined in step b), and e) diagnosing a severe form of cancer or a mild form of cancer.

As used herein, the term “detection agent” relates to an agent specifically interacting with, and thus recognizing, a polypeptide of the present invention, said detection agent being labelled in a way allowing detection of said detection agent inside the human body. Preferably, said detection agent is a polypeptide, e.g. an anticalin, a DARPin, a single-chain T-cell receptor, or an antibody. Preferably, the detection agent is water soluble and can be transported to the tumor tissue via the bloodstream. Also preferably, the detection agent recognizes the polypeptide of the present invention by the presence of said polypeptide or a peptide fragment thereof on the surface of a tumor cell, like e.g. a single-chain T-cell receptor or an antibody specifically recognizing calveolin. Most preferably, the detection agent enters the cell, e.g. by endocytosis, by receptor mediated endocytosis or mediated by a protein transduction domain (e.g. Tat13, Ant16, R13) or the like. Preferably, the label allowing detection of the detection reagent inside the human body is a label as described herein above. More preferably, said label is detectable by computer tomography (CT, e.g. Iodine), by magnet resonance tomography (MRT, e.g. gadolinium), or by positron emission tomography (PET, e.g. ¹⁸F, ^{99m}Tc, ¹¹¹In, ¹³¹I, or ¹⁸⁶Re; van Don et al. (2007), “Immuno-PET: A Navigator in Monoclonal Antibody Development and Applications”, The Oncologist, December 2007 vol. 12 no. 12 1379-1389) and non-invasive tomography methods well known to the skilled artisan.

The term “diagnosing” as used herein refers to assessing the probability according to which a subject is suffering or will suffer from a disease or condition referred to in this specification. As will be understood by those skilled in the art, such an assessment is usually not intended to be correct for 100% of the subjects to be diagnosed. The term, however, requires that a statistically significant portion of subjects can be correctly diagnosed to suffer from the disease or condition. Whether a portion is statistically significant can be determined without further ado by the methods referred to herein above. It is to be understood that the diagnosing of the present invention requires the presence of the subjects at least for steps a) to d), as will be detailed below.

As used herein, the term “applying” a detection agent, preferably, relates to applying said detection agent to the bloodstream of the subject. Preferably, the detection agent is allowed to get distributed in the blood system before the determining and comparing steps of b) and c) are performed. It is, however, also envisaged by the present invention that the detection agent is applied directly to a tissue suspected to comprise tumor cells.

The term “tissue suspected to comprise cancer cells” is understood by the skilled artisan. Preferably, a neoplasm, preferably in the breast, is suspected to comprise cancer cells. It is, however, also envisaged that the complete body of a subject is suspected to comprise cancer cells. The diagnosing will then comprise determining the amount of RPS6, NME/NDKA, and caveolin-1 in the whole body of a subject or at least one part thereof. Likewise, the term “tissue not suspected to comprise cancer cells” is as well understood by the skilled person. The medical practitioner recognizes said tissue not suspected to comprise cancer cells by the absence of neoplasms as evidenced by e.g. CT, MRT, PET, sonography, or radiography, e.g. mammography. Preferably, the tissue suspected to comprise cancer cells and tissue not suspected to comprise cancer cells are tissues from the same subject. More preferably, said tissues are analysed simultaneously.

The determining of the amount of the RPS6, NME/NDKA, and caveolin-1 polypeptides is accomplished by said detection agent of the present invention. Thus, the method of determining the amount of RPS6, NME/NDKA, and caveolin-1 polypeptide in a tissue suspected to comprise cancer cells depends on the label used for the detection agent as specified herein above.

In a further preferred embodiment, the present invention relates to a device for differentiating in a subject with cancer between i) a severe form of cancer and ii) a mild form of cancer, comprising a detection unit for determining the amounts of at least the gene products of claim 1 and an analysing unit for comparing said amounts to reference amounts, allowing differentiating between i) a severe form of cancer and ii) a mild form of cancer

The term “device” as used herein relates to a system of means comprising at least the aforementioned means operatively linked to each other as to allow the differentiation. Preferred means for determining the amount of the said gene products and means for carrying out the comparison are disclosed above in connection with the methods of the invention. How to link the means in an operating manner will depend on the type of means included into the device. For example, where means for automatically determining the amount of the gene products are applied, the data obtained by said automatically operating means can be processed by, e.g., a computer program in order to establish a diagnosis (i.e. identifying a subject being susceptible for the interferon treatment). Preferably, the means are comprised by a single device in such a case. Said device may accordingly include an analyzing unit for the measurement of the amount of the gene products in a sample and an evaluation unit for processing the resulting data for the diagnosis. Alternatively, where means such as test stripes are used for determining the amount of the gene products, the means for diagnosing may comprise control stripes or tables allocating the determined amount to an amount known to be accompanied with response to standard interferon treatment or with non-response to interferon treatment. Preferred means for detection are disclosed in connection with embodiments relating to the methods of the invention above. In such a case, the means are operatively linked in that the user of the system brings together the result of the determination of the amount and the diagnostic value thereof due to the instructions and interpretations given in a manual. The means may appear as separate devices in such an embodiment and are, preferably, packaged together as a kit. The person skilled in the art will realize how to link the means without further inventive skills. Preferred devices are those which can be applied without the particular knowledge of a specialized clinician, e.g., test stripes or electronic devices which merely require loading with a sample. The results may be given as output of parametric diagnostic raw data, preferably, as absolute or relative amounts. It is to be understood that these data will need interpretation by the clinician. However, also envisaged are expert system devices wherein the output comprises processed diagnostic raw data the interpretation of which does not require a specialized clinician. Further preferred devices comprise the analyzing units/devices (e.g., biosensors, arrays, solid supports coupled to ligands specifically recognizing the polypeptides, Plasmon surface resonance devices, NMR spectro-meters, mass-spectrometers etc.) or evaluation units/devices referred to above in accordance with the methods of the invention.

The present invention contemplates a kit comprising instructions to carry out the method of any one of the present invention, and means for determining the amounts of at least the gene products of claim 1, and means for comparing said amounts to reference amounts, allowing differentiating between i) a severe form of cancer and ii) a mild form of cancer.

The term “kit” as used herein refers to a collection of the aforementioned components, preferably, provided separately or within a single container. The container, also preferably, comprises instructions for carrying out the method of the present invention. Examples for such the components of the kit as well as methods for their use have been given in this specification. The kit, preferably, contains the aforementioned components in a ready-to-use formulation. Preferably, the kit may additionally comprise instructions, e.g., a user's manual for interpreting the results of any determination(s) with respect to the diagnoses provided by the methods of the present invention. Particularly, such manual may include information for allocating the amounts of the determined gene products to the kind of diagnosis. Details are to be found elsewhere in this specification. Additionally, such user's manual may provide instructions about correctly using the components of the kit for determining the amount(s) of the respective biomarker. A user's manual may be provided in paper or electronic form, e.g., stored on CD or CD ROM, or downloadable via a web-interface from an online repository. The present invention also relates to the use of said kit in any of the methods according to the present invention.

All references cited in this specification are herewith incorporated by reference with respect to their entire disclosure content and the disclosure content specifically mentioned in this specification.

### FIGURES

FIG. 1: Box plot diagrams showing expression of A) RPS6, NME/NDKA, B) Ki-67, TOP2A, and C) caveolin-1 in 109 breast tumors with histologic grading 1 (G1) and grading 3 (G3).

FIG. 2: Box plot diagrams showing expression of A) RPS6, NME/NDKA, B) Ki-67, TOP2A, C) caveolin-1 and estrogen receptor alpha (ESR1) in 109 breast tumors with histologic grading 1 (G1), grading 2 (G2) and grading 3 (G3). ESR1 expression was included as control and did not reveal grading-dependent differences.

FIG. 3: Three-marker heatmap reflecting the abundance of caveolin-1, RPS6, and NME/NDKA for a set of 109 estrogen receptor positive human breast tumors. Of these 109 tumors, 18 were classified by histology as low risk (G1) and 22 as high risk tumors (G3).

FIG. 4: Five-marker heatmap reflecting the abundance of caveolin-1, RPS6, NME/NDKA, Ki-67, and TOP2A for a set of 109 estrogen receptor positive human breast tumors. Of these 109 tumors, 18 were classified by histology as low risk (G1) and 22 as high risk tumors (G3).

FIG. 5: A, Examples for NDKA immunohistochemistry (IHC). Group 1 (IHC 1) represents cases with no or low immunoreactivity, group 2 (IHC 2) intermediate cases, and group 3 (IHC 3) tumors with diffuse and strong NDKA expression. B, Protein expression of NDKA and Ki-67 measured using RPPA correlates with the respective immunohistochemistry data, Kruskal-Wallis test, p<0.001.

FIG. 6: IHC evaluation of biomarker expression. Representative IHC images of caveolin-1, NDKA, RPS6, and Ki-67 for samples classified by RPPA either as low risk (left) or high risk (right) are shown. High caveolin-1 expression was observed in the tumor microenvironment in case of low risk patients, whereas high expression of NDKA, RPS6, and Ki-67 was present in tumor cells of high risk patients.

FIG. 7: Comparison of biomarker protein and mRNA expression levels. A, Correlation of protein and mRNA expression derived by RPPA and Illumina whole genome gene expression profiling and RPPA, respectively. A significant correlation was observed for caveolin-1, NDKA, and Ki-67 (p<0.001, Spearman's rank correlation) but not for RPS6. B, Association of high NDKA (NME1) and Ki-67 (MKI67) mRNA expression with histologic G3 tumor samples as well as high caveolin-1 (CAV1) mRNA expression of histologic G1 tumor samples was confirmed using the independent sample set (estrogen receptor positive tumor samples, n=406) of Curtis et al. (Curtis et al. (2012), The genomic and transcriptomic architecture of 2,000 breast tumours reveals novel subgroups. Nature 486, 346-352).

### EXAMPLES

The following Examples shall merely illustrate the invention. They shall not be construed, whatsoever, to limit the scope of the invention.

### Example 1

The tumor set comprised 15 “grade 1” and 22 histologic “grade 3” tumor specimens as well as 72 histologic “grade 2” tumors. Tumor samples were cut into 60 μm slices using a cryomicrotome. Aliquots were homogenized using a bead mill and T-PER® lysis buffer supplemented with phosphatase, kinase, and protease inhibitors. Tumor lysates were adjusted to a total protein concentration of 2 μg/μl and mixed with sample buffer containing SDS and DTT. Samples were heated to 95° C. prior to spotting and three technical replicates were deposited on nitrocellulose coated glass slides. In addition, control cell lines (MDA-MB-231, MCF7, HCC1954) were printed as dilution series. Lysis buffer was used as negative control. Representative slides were stained with Fast Green FCF® for total protein quantification for spot normalization (Loebke et al). Antibodies recognizing 128 different proteins and phosphoproteins that are known to be implicated in breast cancer (Table 2) were used for detection and visualized using secondary antibodies labeled with the fluorescent dye Alexa-680. Signals were visualized on a near infrared fluorescence scanner. Signal intensities were determined using the Genepix software and analyzed relying on in house software (Mannsperger H A, Gade S, Henjes F, Beissbarth T, Korf U. RPPanalyzer: Analysis of reverse-phase protein array data. Bioinformatics, 2010, 26, 2202-3).

TABLE 2
Targeted proteomics for 128 breast cancer relevant targets

| | | | | |
|---|---|---|---|---|
| Acetyl_CoA_Carboxylase | N-cadherin | pRB_S807_S811 | AKT1 | NFkB |
| new | | | | |
| pRPS6_S235_S236 | AKT2 | NME1_NDKA | pRPS6_S240_S244 | APC |
| NOTCH2 | pSRC_Y416 | ATM | NOTCH3 | pSTAT1_Y701 |
| ATR | p27 | pSTAT3_Y705 | BAX | p38 |
| pSTAT5_Y694_Y699 | bCatenin_new | p53 | PTEN | BCL-2 |
| p70S6K_2708 | pTSC2_T1462 | BCL-XL | PAK1 | pyruvate_dehydrogenase |
| BRCA2 | PAK2 | RB | caveolin_1 | pAKT_S473 |
| RKIP | CBL | pAKT_T308 | ROCK1 | CDK1 |
| PARP | ROCK2 | CDK2 | pbCatenin_S33_S37_T41 | RPS6 |
| CDK6 | P-cadherin | RSK | cJUN | PCNA |
| SDHA | Claudin-1 | pcRAF_S259 | SHP1 | Claudin-3 |
| PDI | SHP2 | COL4A3BP | PDK1 | SMAD2 |
| CREB | pERBB2_Y1112 | SMAD7 | Cyclin_B1 | pERBB2_Y1248_ab47755 |
| SMURF2 | Cyclin_D1_sc-718 | pERBB4_Y1162 | SRC | Cytokeratin_18 |
| pERK1_pERK2_T202_Y204 | STARD10 | Cytokeratin_8_S23 | pFAK_S843 | STAT1 |
| E-cadherin | pFoxO3a_S318_S3 | STAT3 | EGFR | pGSK3A_S21 |
| | 21 | | | |
| TIE2_ | EpCAM_neu | pGSK3A_Y279_pGSK3B_Y216 | TOP2A | ERa |
| pGSK3B_S9 | TSC1 | ERBB2 | PI3K_p110_4249 | TSC2 |
| ERBB3 | PI3K_p85 | VEGFR2 | ERBB4 | PKA |
| Vimentin | ERK1 | PKCa | FIH | PLCg |
| GATA3 | pMEK_S217_S221 | GRB2 | pmTOR_S2448 | GSK3A |
| pNFkB_S536 | GSK3B | pp38_T180_Y182 | IntegrinB1 | pp53_Ser15 |
| IntegrinB3 | pp70S6K_T389 | Ki67 | pp70S6K_T421_S424 | LAMB1 |
| pp90RSK_S380 | MCL-1 | pPDK1_S241 | MEK | pPKCa_S657_Y658_ab235 |
| | | | | 13 |
| MET_3148 | pPRAS40_T246 | metadherin | pPTEN_T366_S370 | MNK1 |
| PR | mTOR_2983 | PRAS40 | | |

### Example 2

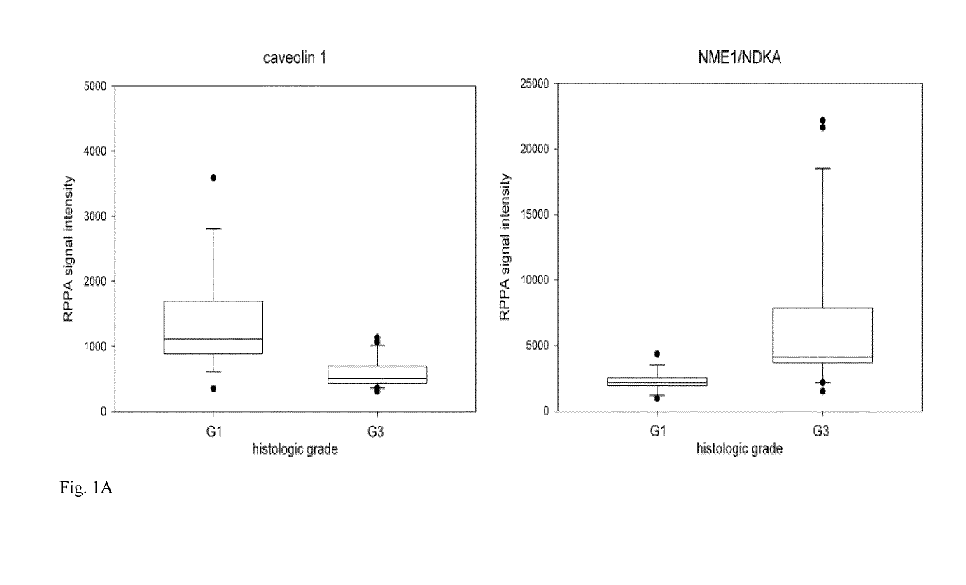

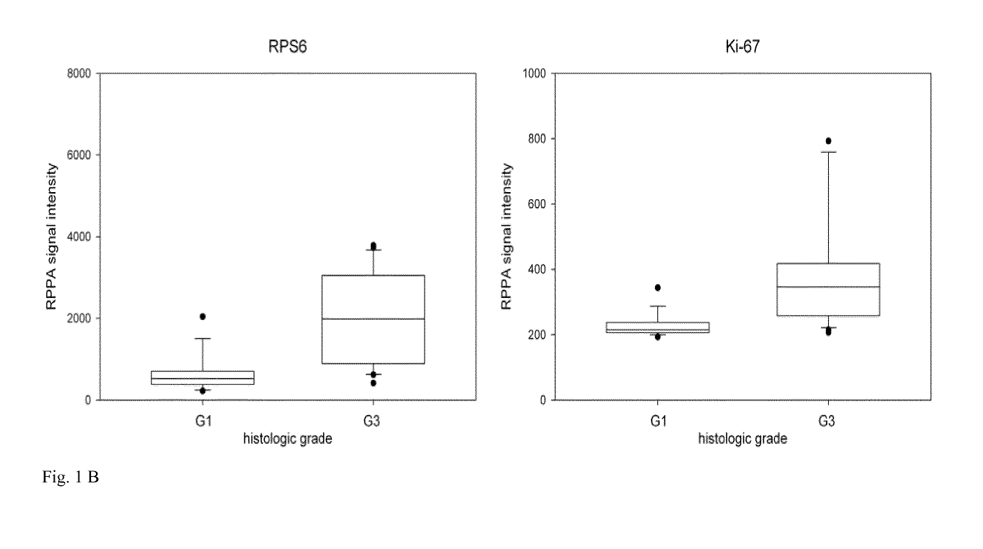

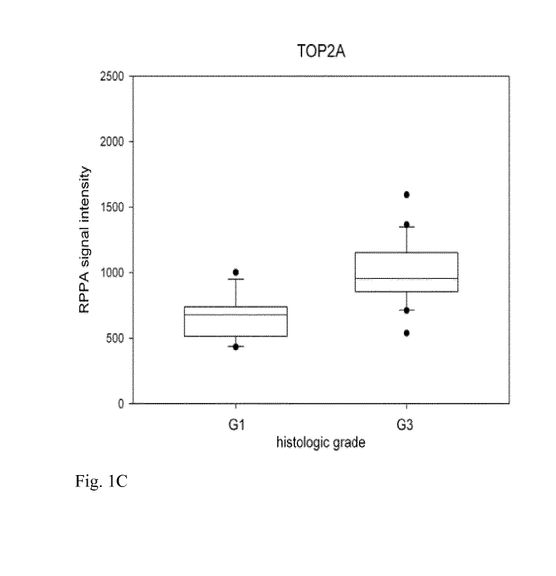

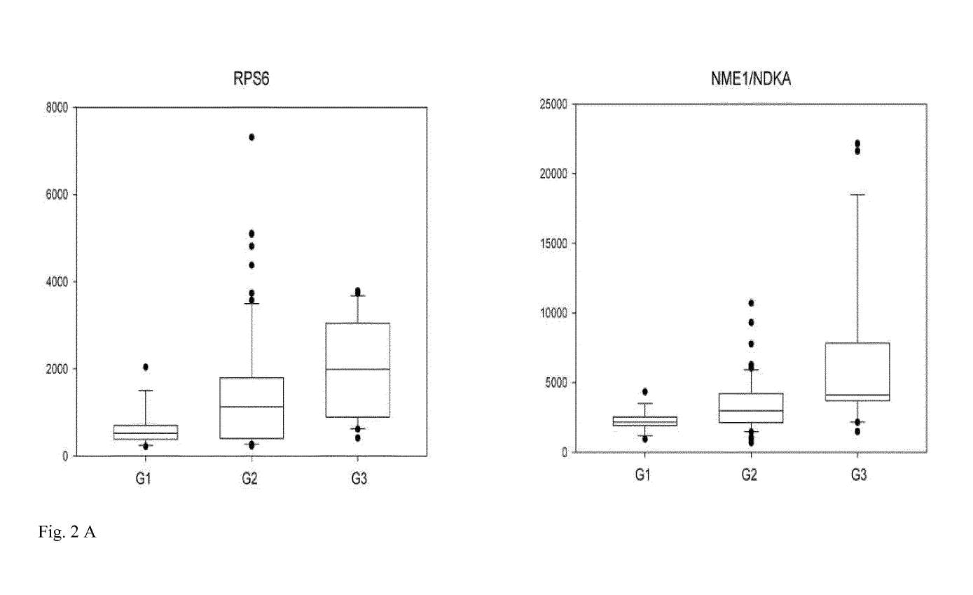

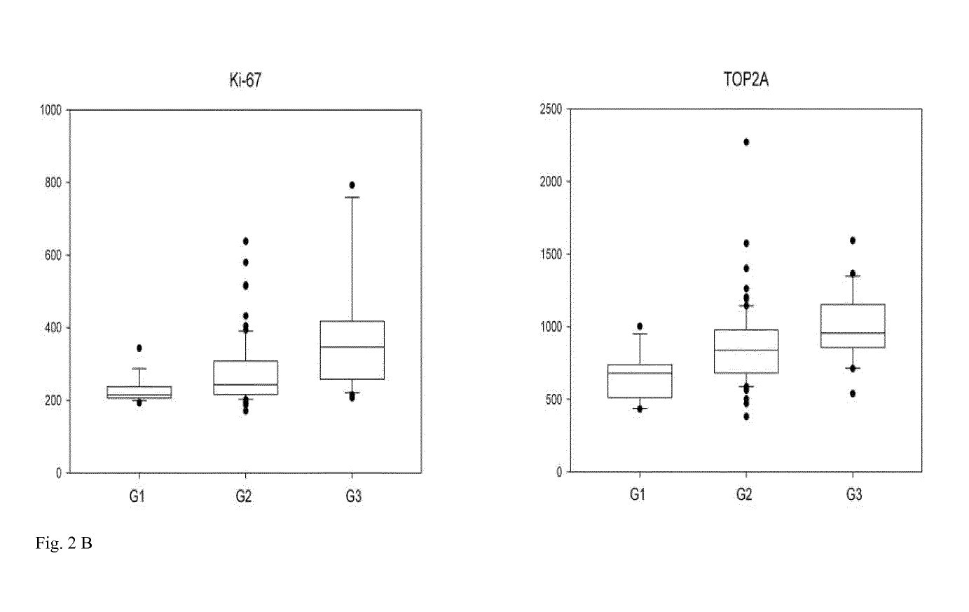

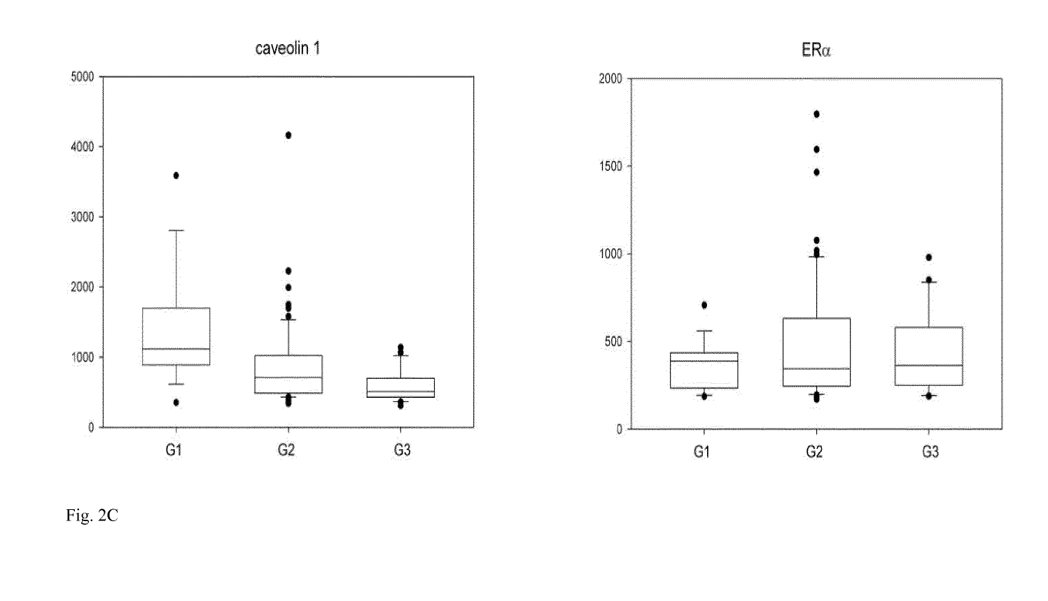

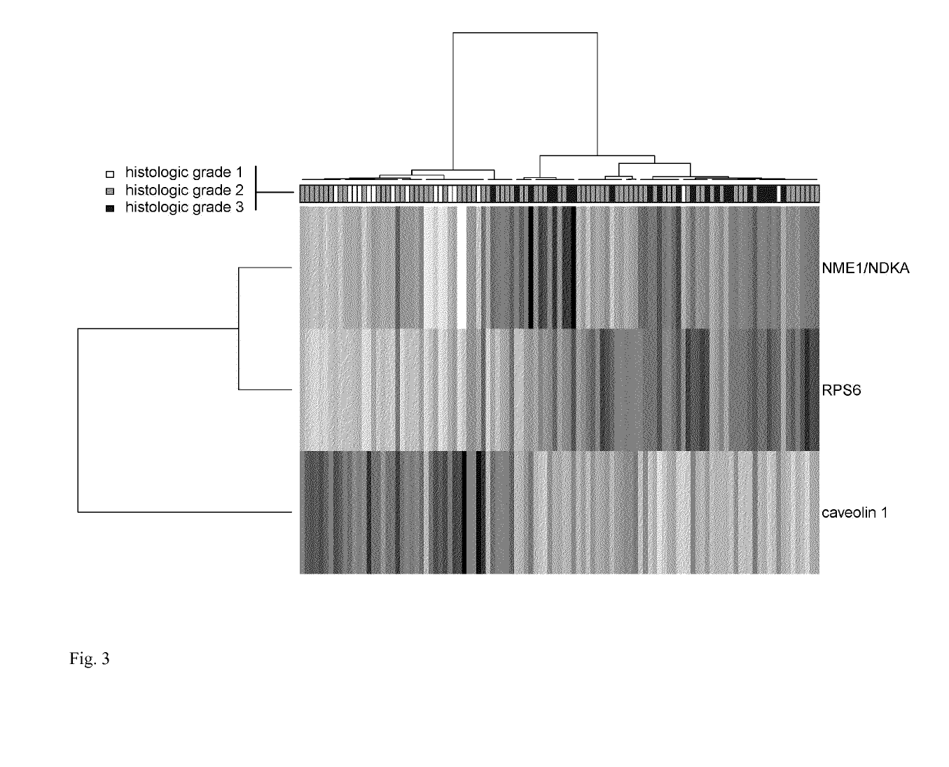

To identify proteins differentially regulated between “grade 1” and “grade 3” tumor samples the corresponding data was analyzed using a combination of three different classification algorithms, in detail, SVM, random forest, and PAM (Becker, N., Werft, W., Toedt, G., Lichter, P., and Benner, A. (2009) penalizedSVM: a R-package for feature selection SVM classification, Bioinformatics 25, 1711-1712. //Kursa, M. B., and Rudnicki, W. R. (2010) Feature Selection with the Boruta Package., Journal of Statistical Software 36, 1-13. //Tibshirani, R., Hastie, T., Narasimhan, B., and Chu, G. (2002) Diagnosis of multiple cancer types by shrunken centroids of gene expression, Proc Natl Acad Sci USA 99, 6567-6572). This approach identified caveolin-1, NME1/NDKA, and RPS6 (FIG. 1 boxplots G1 vs G3) as top candidate proteins that can discriminate between “grade 1” and “grade 3” tumors. In addition, Ki-67 and TOP2A were also among the top 10 candidates (FIG. 2). Signal intensities for the top three candidates (caveolin-1, NME1/NDKA, RPS6) were analyzed for the full data set including also those samples classified as histologic “grade 2”. These samples aligned with “grade 1” or “grade 3” tumors and did not form an independent cluster (FIG. 3). This suggests that a 3-marker set is sufficient to allow a risk classification of breast cancer patients with hormone receptor positive tumors of intermediate grading.

### Example 4

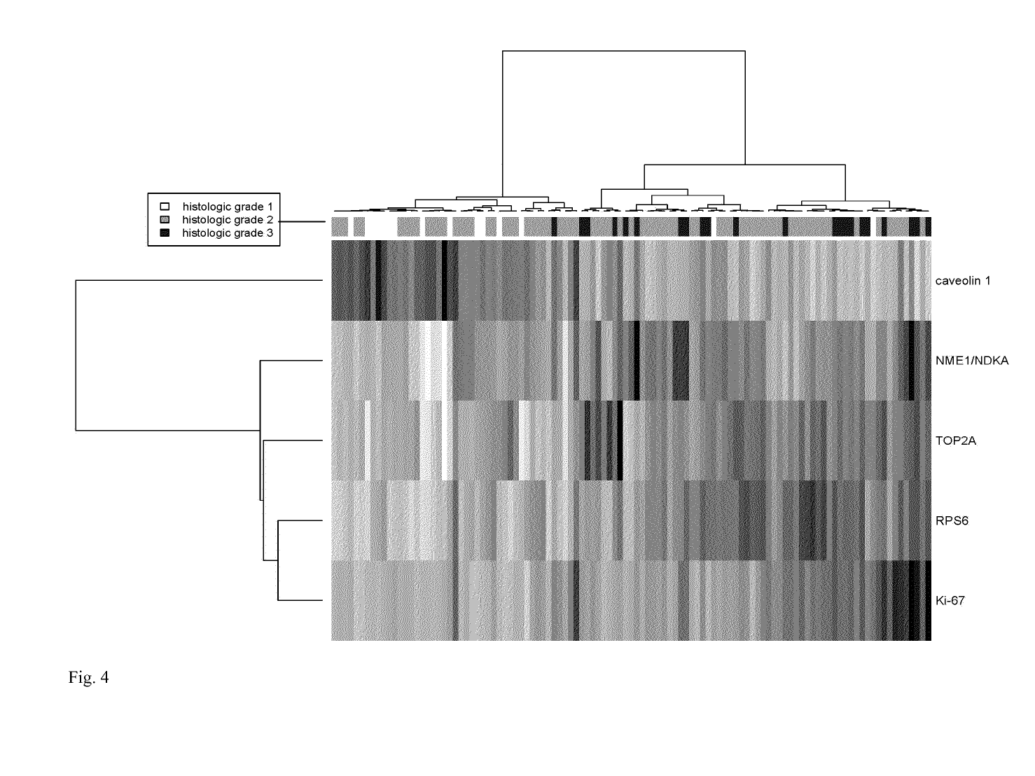

For most biomarkers, decision-making has to compromise between high sensitivity and high specificity resulting in the fact that patients are either overtreated or suffer from early relapses. This exact situation has pertained treatment decision for patients with hormone receptor positive breast cancer for long periods of time. As shown in FIG. 4, relying on MKI67 or TOP2A as a single marker would result in undertreatment of patients whose tumors express high levels of NME/NDKA or RPS6 but only low levels of MKI67 or TOP2A. This affects approximately 10-20% of hormone-positive breast cancer patients. A downregulation of caveolin-1 in the tumor stroma was identified by us as highly synergistic marker when assessed in combination with at least two other proliferation markers such as 5-marker combination RPS6, NME/NDKA, Ki67, TOP2A (FIG. 4) or as 3-marker combination (FIG. 3, Table 3).

Caveolin-1, NDKA, and RPS6 followed by Ki-67 were the most important proteins to discriminate between histologic G1 and G3 patients. This finding was visualized by hierarchical cluster analysis (FIGS. 3 and 4) which separated the 36 samples in two main groups comprising either histologic G1 or histologic G3 samples. Only two tumors were misclassified.

Protein expression levels of caveolin-1, NDKA, RPS6, and Ki-67 were next assessed by including RPPA data obtained for histologic G2 samples (n=73). The cluster analysis revealed that histologic G2 samples cover the full expression level range and do not form a distinct group with respect to the expression of the four biomarker proteins (FIGS. 3 and 4). This suggests that histologic G2 patients with high level expression of NDKA, RPS6 or Ki-67 as well as low level expression of caveolin-1 are at high risk for relapse as their protein biomarker profile is highly similar to that of histologic G3 patients. To assign histologic G2 samples either as being at low or high risk of cancer relapse according to the surrogate marker profile, a risk classification score named R2LC (RPPA Risk Linear Classification) was developed. This score is a weighted linear combination of individual biomarker expression levels which was derived by a bootstrapped linear model fit regressing histologic grade onto protein expression data of selected markers. The bootstrapped estimate of the score was derived as:

[R2LC]=−0.464*[caveolin-1]+0.266*[NDKA]+0.194*[RPS6]+0.208*[Ki-67] (formula I).

Thus, if R2LC>2 the sample is categorized as high risk and if R2LC<2 as low risk. The performance of R2LC to classify independent test sets was assessed additionally in a 5-fold cross validation with 10 repeats, showing good performance with AUC=0.987. Using R2LC, 25 out of 73 histologic G2 patients were classified as low risk whereas the other 48 patients were classified as being at high risk of recurrence. Again, two main clusters with either histologic G1 or histologic G3 samples resulted whereas histologic G2 samples were distributed between both main clusters. These two main clusters also reflect the low risk and the high risk group as identified by applying the risk classification score R2LC.

TABLE 3
Normalized signal intensities top-3-markers

| Tumor | Hist. | Caveolin 1 | RPS6 | NME1_NDKA |
|---|---|---|---|---|
| no. | Grading | Signalint. | Signalint. | Signalint. |
| 12 | 2 | 1258 | 274 | 1467 |
| 16 | 2 | 702 | 468 | 2171 |
| 23 | 2 | 1422 | 267 | 1621 |
| 26 | 3 | 1137 | 680 | 4977 |
| 41 | 1 | 2282 | 257 | 2048 |
| 43 | 2 | 957 | 407 | 2168 |
| 44 | 2 | 1580 | 288 | 1683 |
| 45 | 2 | 471 | 260 | 914 |
| 55 | 2 | 832 | 233 | 1463 |
| 58 | 1 | 968 | 522 | 2534 |
| 72 | 1 | 1386 | 224 | 943 |
| 78 | 1 | 1669 | 485 | 1993 |
| 85 | 1 | 1136 | 384 | 2257 |
| 89 | 2 | 899 | 523 | 2147 |
| 92 | 2 | 1075 | 262 | 3040 |
| 95 | 2 | 1105 | 374 | 2437 |
| 98 | 2 | 991 | 417 | 2936 |
| 102 | 2 | 1697 | 324 | 1887 |
| 115 | 1 | 890 | 440 | 2940 |
| 127 | 2 | 1111 | 354 | 2563 |
| 129 | 2 | 2230 | 2458 | 4915 |
| 138 | 1 | 1048 | 323 | 2211 |
| 141 | 2 | 927 | 414 | 1768 |
| 145 | 1 | 998 | 574 | 1927 |
| 154 | 2 | 899 | 1039 | 3024 |
| 155 | 2 | 1304 | 1989 | 6052 |
| 161 | 1 | 3589 | 639 | 1503 |
| 165 | 2 | 853 | 392 | 2513 |
| 169 | 2 | 1414 | 376 | 1897 |
| 181 | 2 | 887 | 611 | 4004 |
| 185 | 2 | 941 | 715 | 4186 |
| 186 | 1 | 1698 | 708 | 1343 |
| 191 | 2 | 1163 | 773 | 3703 |
| 196 | 2 | 1707 | 250 | 684 |
| 207 | 1 | 1867 | 870 | 2408 |
| 216 | 2 | 4163 | 388 | 683 |
| 217 | 2 | 469 | 274 | 2115 |
| 220 | 2 | 1393 | 357 | 1509 |
| 221 | 1 | 1116 | 689 | 2840 |
| 229 | 2 | 1750 | 300 | 1085 |
| 237 | 2 | 1992 | 1732 | 6137 |
| 240 | 2 | 1064 | 407 | 987 |
| 244 | 1 | 792 | 458 | 2176 |
| 255 | 2 | 902 | 388 | 4231 |
| 77_L | 2 | 923 | 964 | 2847 |
| 8 | 3 | 915 | 2914 | 4947 |
| 15 | 2 | 681 | 3579 | 3365 |
| 29 | 2 | 436 | 1328 | 4984 |
| 49 | 2 | 676 | 1120 | 2722 |
| 50 | 2 | 538 | 1257 | 3775 |
| 52 | 3 | 514 | 1227 | 9976 |
| 54 | 2 | 377 | 931 | 7786 |
| 68 | 3 | 503 | 651 | 2219 |
| 69 | 2 | 544 | 1775 | 4471 |
| 71 | 3 | 1066 | 910 | 22164 |
| 73 | 2 | 584 | 747 | 2998 |
| 74 | 2 | 511 | 422 | 6285 |
| 75 | 1 | 354 | 1150 | 1983 |
| 76 | 3 | 409 | 1898 | 4456 |
| 84 | 2 | 389 | 5109 | 3187 |
| 91 | 2 | 424 | 1658 | 3560 |
| 97 | 3 | 481 | 1995 | 3771 |
| 99 | 2 | 488 | 681 | 2971 |
| 105 | 3 | 529 | 2441 | 11195 |
| 109 | 2 | 831 | 1542 | 2042 |
| 110 | 2 | 713 | 788 | 3898 |
| 111 | 3 | 360 | 3537 | 8926 |
| 114 | 2 | 451 | 610 | 2099 |
| 118 | 3 | 309 | 3520 | 7495 |
| 119 | 2 | 400 | 1332 | 4472 |
| 120 | 3 | 864 | 3464 | 21629 |
| 122 | 3 | 428 | 1703 | 3982 |
| 124 | 3 | 536 | 619 | 2157 |
| 125 | 3 | 461 | 416 | 3807 |
| 126 | 2 | 749 | 1691 | 4245 |
| 133 | 2 | 1004 | 1375 | 2792 |
| 137 | 2 | 540 | 1213 | 2217 |
| 140 | 3 | 453 | 2467 | 3782 |
| 147 | 3 | 768 | 1127 | 3459 |
| 151 | 1 | 798 | 2042 | 4343 |
| 157 | 2 | 511 | 1493 | 3111 |
| 164 | 2 | 705 | 1365 | 2658 |
| 167 | 3 | 478 | 2432 | 1495 |
| 172 | 2 | 485 | 5092 | 3177 |
| 176 | 2 | 478 | 1870 | 3295 |
| 179 | 2 | 808 | 1906 | 5579 |
| 187 | 3 | 433 | 2350 | 4232 |
| 188 | 3 | 372 | 3789 | 4412 |
| 189 | 2 | 478 | 1141 | 10716 |
| 190 | 3 | 511 | 848 | 2601 |
| 193 | 2 | 1028 | 3226 | 6227 |
| 203 | 3 | 679 | 3733 | 3865 |
| 206 | 2 | 602 | 4380 | 4857 |
| 208 | 2 | 644 | 1431 | 2336 |
| 209 | 2 | 466 | 808 | 1783 |
| 224 | 2 | 449 | 1804 | 3874 |
| 227 | 2 | 502 | 1371 | 3452 |
| 233 | 2 | 341 | 7313 | 4305 |
| 234 | 2 | 431 | 1301 | 9315 |
| 235 | 2 | 604 | 1899 | 5231 |
| 239 | 2 | 562 | 3737 | 4412 |
| 241 | 3 | 549 | 1981 | 3787 |
| 243 | 2 | 719 | 2534 | 2845 |
| 249 | 2 | 447 | 3309 | 3925 |
| 251 | 2 | 523 | 3287 | 2934 |
| 252 | 2 | 771 | 1430 | 2397 |
| 258 | 2 | 595 | 4813 | 2518 |
| 261 | 2 | 451 | 1664 | 1586 |
| 77_R | 2 | 353 | 2994 | 3541 |

Converting the 5-biomarker panel into an assay compatible with the daily routine in immunohistology requires the definition of suitable cut-off values for a combinatory readout. In detail, this can be achieved by using reverse phase protein microarrays as experimental platform. For this reason, we use a tailored protein array that contains several subarrays with a serial dilution of the 5-marker panel proteins as well as other breast cancer relevant proteins such as estrogen receptor, progesterone receptor, ERBB2 and EGFR. This protein array can be stored and taken from the fridge to take up a few-step serial dilution containing an individual patient tumor sample. A suitable frame generating incubation wells is mounted on top of the slide and each well is incubated with a target-protein specific antibody, detected, and signals are quantified. Relying on signals generated by standard curves the abundance of a specific protein in a certain tumor can be determined. This platform generates quantitative information on the expression predictive breast-cancer proteins in tumor samples (e.g. luminal breast cancer).

### Example 5

### Evaluation of Identified Biomarkers Using Immunohistochemistry

Immunohistochemistry (IHC): Immunohistochemical Ki-67 staining was performed using an automated staining system (Techmate 500, DakoCytomation). Primary antibody Ki-67 (MIB-1, 1:200, DakoCytomation) was used after pretreatment with microwave/citrate buffer. All IHC stained slides were analyzed after virtual microscopy scanning at 20× (Aperio Technologies). For the negative control, the primary antibody was omitted. Immunostains of tissue microarrays (TMA) were carried out using primary antibodies against caveolin-1 (610407, BD Biosciences, 1:100), NDKA (5353, Cell Signaling Technologies, 1:300), and RPS6 (2217, Cell Signaling Technologies, 1:200). Briefly, 1-2 μM sections of the TMAs were deparaffinized using xylene and rehydrated in a series of graded alcohols. Heat-pretreatment was performed in 1 mM EDTA (pH 8.0) in a water bath at 95° C. for 30 minutes followed by incubation with the respective primary antibody at 4° C. over night. Antibody binding was detected using a modified avidin-biotin-complex method with horseradish peroxidase and 3-aminoethylcarbazol (AEC) as chromogen (DAKO Chemmate, Dako, Hamburg, Germany).

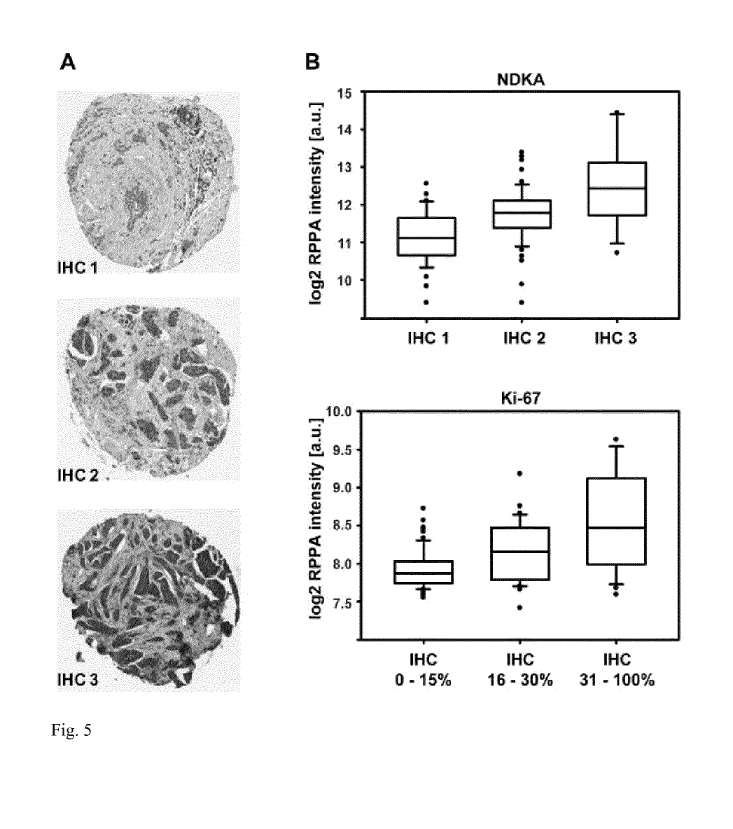

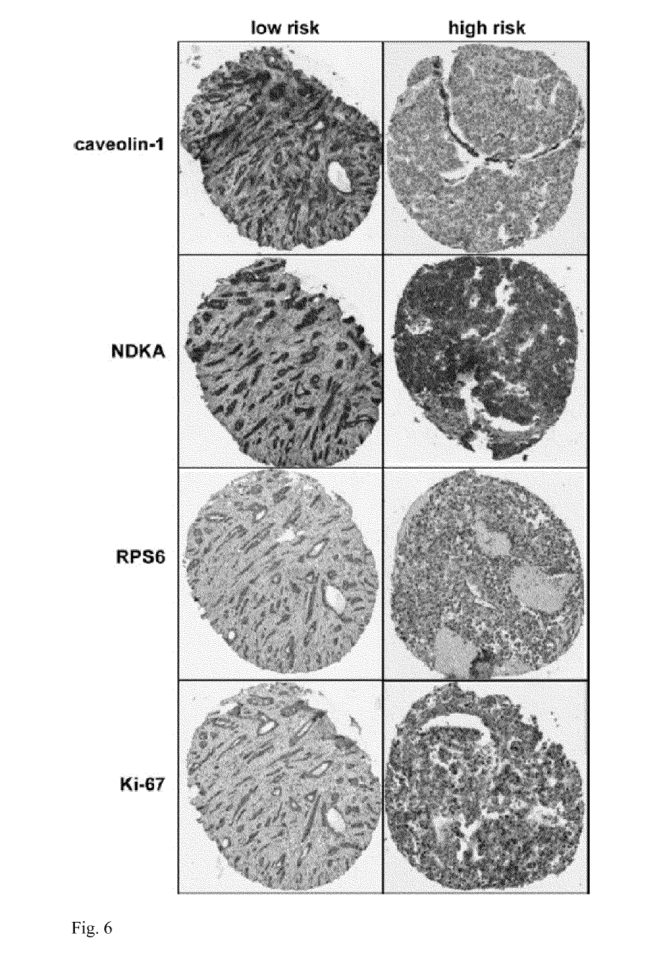

Evaluation: To further validate the RPPA derived results, immunohistochemistry (IHC) was carried out for caveolin-1, NDKA, and RPS6, using tissue microarrays comprising a large number of tumors also analyzed by RPPA. Ki-67 data was available for 103 of 109 patients since this marker was assessed routinely in the clinics. Ki-67 staining was observed in the nucleus of tumor cells but with varying degree between patients. A significant correlation was obtained for the comparison of Ki-67 RPPA data and IHC grouped patients with low (0%-15%), medium (16%-30%) or high (31%-100%) Ki-67 staining (p<0.001, Kruskal-Wallis test, FIG. 5B). Staining of tissue microarrays with antibodies directed against NDKA, caveolin-1, and RPS6 was available for a subset of 96 patients. NDKA mainly localized to the cytoplasm of tumor cells with low staining intensity in 33 samples, medium staining intensity in 49 samples, and high staining intensity in 14 samples (FIG. 5A). Protein expression of NDKA as measured by RPPA correlated significantly with the IHC scoring (p<0.001, Kruskal-Wallis test, FIG. 5B). Caveolin-1 expression was mainly observed in the tumor stroma. Loss of caveolin-1 in cancer-associated fibroblasts was seen in samples classified by RPPA as being at high risk. RPS6 was located in the cytoplasm of tumor cells. In few samples, infiltrating immune cells as part of the tumor microenvironment stained also strongly for RPS6. FIG. 6 shows two representative cases, one classified by RPPA as being at low risk and one as high risk, supporting the RPPA derived score which suggested low caveolin-1 staining but high level expression of NDKA, RPS6, as well as Ki-67 in high risk tumors and a reversed staining pattern for low risk tumors.

### Example 6

### Comparison of Biomarker Protein and mRNA Expression Levels

Transcriptional profiling: Total RNA was isolated from tumor samples (n=71) using the miRNeasy Mini kit (Qiagen) according to manufacturer's instructions. Quality control of total RNA as well as labeling and hybridization to Sentrix Human HT-12 v4 BeadChips (Illumina) were performed at the DKFZ Proteomics and Genomics core facility. Transcriptional profiling data were log-transformed and quantile normalized. For validation, a subset of the discovery cohort published by Curtis et al. (Curtis et al. (2012), The genomic and transcriptomic architecture of 2,000 breast tumours reveals novel subgroups. Nature 486, 346-352) consisting of 406 estrogen receptor positive breast cancer samples (only histologic grade 1 and grade 3) was used.

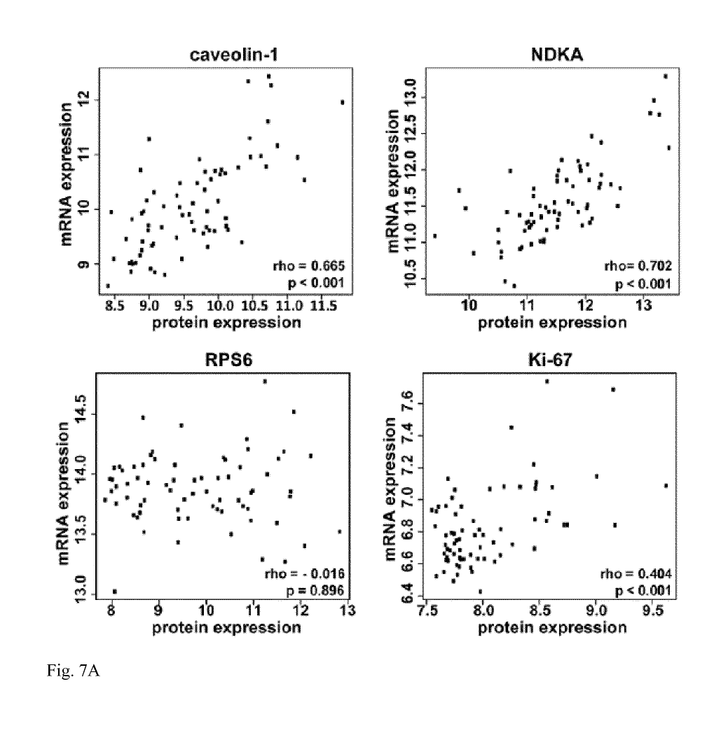

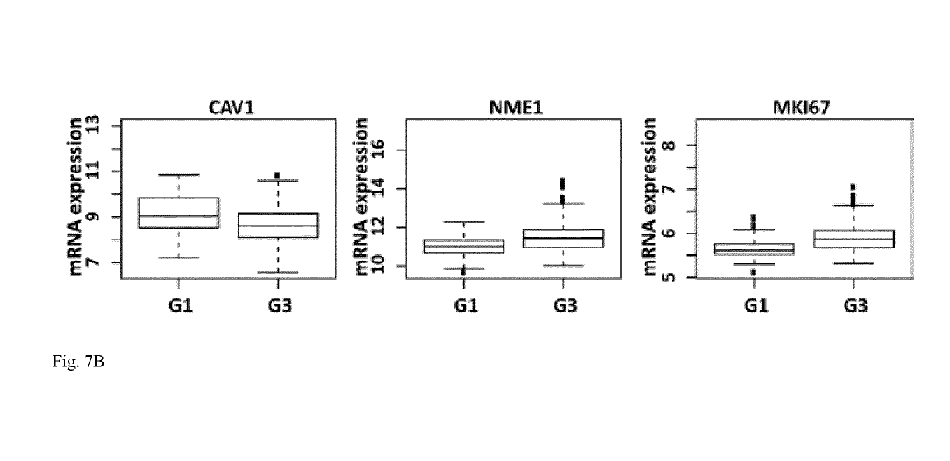

Comparison of biomarker protein and mRNA expression levels: To evaluate the selected biomarker set consisting of caveolin-1, NDKA, RPS6, and Ki-67 further, a comparison of mRNA and protein expression was carried out for a subset of 71 samples. Correlation analysis revealed that caveolin-1 mRNA and protein level were positively correlated (p<0.001) with a Spearman correlation coefficient of p=0.665. NDKA and Ki-67 also had a significant positive correlation with p=0.702 and p=0.404, respectively. In case of RPS6, no correlation between mRNA and protein expression was observed (FIG. 7A). The recently published data set of Curtis et al. (ibd.) comprising 406 estrogen receptor positive breast cancer samples was used to compare gene expression levels of caveolin-1, NDKA, and Ki-67 with the annotated histologic grading status. In line with RPPA derived results, mRNA levels of caveolin-1 were significantly higher in histologic G1 samples compared to G3 samples. In addition, NDKA and Ki-67 revealed a higher expression in histologic G3 samples (FIG. 7B).

TABLE 4
Patient characteristics of the study cohort (n = 109).
Median age of the study cohort was 64 years (range 31-86).

| characteristic | number of patients | % |
|---|---|---|
| pT category (UICC2009) | | |
| pT1 | 44 | 40 |
| pT2 | 55 | 50 |
| pT3 | 6 | 6 |
| pT4 | 4 | 4 |
| lymph node status^{a} | | |
| positive | 38 | 35 |
| negative | 70 | 64 |
| histologic grade | | |
| 1 | 14 | 13 |
| 2 | 73 | 67 |
| 3 | 22 | 20 |
| ERα status | | |
| positive | 109 | 100 |
| negative | 0 | 0 |
| PR status | | |
| positive | 102 | 94 |
| negative | 7 | 6 |
| HER2 status | | |
| positive | 4 | 4 |
| negative | 105 | 96 |

^{a}Numbers do not add up to 109 due to data missing for one patient.

1-21. (canceled)

22. A method for differentiating between a severe form of cancer and a mild form of cancer, comprising:
(a) determining the amounts of gene product of at least the genes coding for ribosomal protein S6 (RPS6), nucleoside diphosphate kinase (NME/NDKA), and caveolin-1 in a sample from a subject,
(b) comparing the amounts obtained in step (a) to reference amounts, and
(c) differentiating between a severe form of cancer and a mild form of cancer, wherein an increased amount of product of the gene coding for RPS6 and an increased amount of product of the gene coding for NME/NDKA and a decreased amount of product of the gene coding for caveolin-1 are indicative of a severe form of cancer.

23. The method of claim 22, wherein:
the method in step (a) further comprises determining the amount of gene product of the gene coding for KI-67, and/or the amount of gene product of the gene coding for DNA topoisomerase 2-alpha (TOP2A); and
(ii) step (b) further comprises comparing the amounts to reference amounts;
(iii) in step (c) an increased amount of product of the gene coding for KI-67 and/or of the gene coding for TOP2A is further indicative of a severe form of cancer.

24. The method of claim 22, wherein at least one of the gene products is a polypeptide.

25. The method of claim 22, wherein the gene products are polypeptides.

26. The method of claim 24, wherein the polypeptides are determined by a reverse phase protein array (RPPA), immunohistochemistry or by an antibody array.

27. The method of claim 22, wherein the sample is a tumor sample.

28. The method of claim 22, wherein the cancer is breast cancer.

29. The method of claim 22, wherein the cancer is hormone receptor positive breast cancer.

30. The method of claim 22, wherein the cancer is hormone-receptor positive breast cancer with intermediate histologic grading.

31. The method of claim 22, wherein the mild form of cancer is a cancer not necessitating chemotherapy and wherein the severe form of cancer is a cancer necessitating chemotherapy.

32. The method of claim 22, wherein the mild form of cancer is a cancer with a high probability to respond to anti-estrogen therapy.

33. A method for diagnosing a severe form of cancer or a mild form of cancer in a subject, wherein the use comprises:
(a) applying a detection agent specifically recognizing a polypeptide selected from the group consisting of RPS6, NME/NDKA, and caveolin-1 to the subject or to a sample of the subject;
(b) determining the amount of RPS6, NME/NDKA, or caveolin-1 in a tissue of the subject suspected to comprise cancer cells or in the sample;
(c) comparing the amount determined in step (b) to a reference amount determined from tissue or from a sample not suspected to comprise cancer cells;
(d) determining a difference between the amount determined in step (b) and the amount determined in step (c); and
(e) diagnosing a severe form of cancer or a mild form of cancer.

34. The method of claim 33, wherein the detection agent is an antibody, an anticalin, a Designed Ankyrin Repeat Protein (DARPin), or a single-chain T-cell receptor.

35. A device for differentiating in a subject with cancer between a severe form of cancer and a mild form of cancer, wherein the device comprises:
(a) means for determining the amounts of at least the gene products of claim 22; and
(b) means for comparing the amounts to reference amounts, allowing the differentiation between a severe form of cancer and a mild form of cancer.

36. A kit or an array comprising:
(a) an antibody specifically recognizing the RPS6 polypeptide, an antibody specifically recognizing the NME/NDKA polypeptide, and an antibody specifically recognizing the caveolin-1 polypeptide; or
(b) instructions to carry out the method of claim 22, and means for determining the amounts of at least the gene products of claim 22, and means for comparing the amounts to reference amounts, allowing differentiation between a severe form of cancer and a mild form of cancer.

37. The kit or array of claim 36, further comprising an antibody specifically recognizing the KI-67 polypeptide and/or an antibody specifically recognizing the TOP2A polypeptide.


SEQUENCE LISTING (15 sequences)

SEQ ID NO: 1 - DNA, 3982 bp, Homo sapiens
cctcttttccgtggcgcctcggaggcgttcagctgcttcaagatgaaggtaggtgatggtggcgagtgttagactgggtttggggaacgtgaatcgagtcccagaacgcggcattgcctcagttccagcactccaggatcctggctttaggtggagaagggtctcaagtaggagaaggctcgcctttctggggcatggagctttttggccgaacggatggcaggcgattgcggctggagccgcggcgggccgggagcgccatggtggcgtccccgcgcccagccgggacggatgcggcgtgttgcccagtttgcggcaggcctgtggtgcggctcttgaccccggctttcttgcttcgggagggtgaacggctgcggagtgccctcgcccccagagtcattccgcggggcttgaggggaaaacgtcctgctgagtgcggcgcttcttgactgctactctgctttcacgtgcttttagtgagtacagtcggcatcttatatttcctgcttgtgtggaggcaacatgaaaggctttttgcagtggaattaactttgtagatggctctacaattacctgtatagatagtttcgtaaactatttccccccttttaatccttagctgaacatctccttcccagccactggctgccagaaactcattgaagtggacgatgaacgcaaacttcgtactttctatgagaagcgtatggccacagaagttgctgctgacgctctgggtgaagaatggaaggtaaaagttgacaaattgttgcaggtatttaagtcagagacggtaaacgccattggtaactggtatttggaatggggttcagactccgggttctggcttctgacctttggtaagttgcttccgaatgccactttataaagttagaggtattaccttggagggggggacgtagagtaagccataaaatatacgtaaagtttacatcaacataattcttgccctgcatcatgcatttggcaatatgtcacatagctgtcctcataatccccaaagtgccaaaaagggttgtatctgatttgtttgttgctgtttgatattttatcttcttagtgctgttatctctaataaaacatcggttagaaatgcgacttgaaagacatttgataattgaacttgacaagttgggaatatagacaaaactcactgaacagagaaaacgtgcttaagttcaaattggttttcataattatactttctcatataggtgtgttcacagaggtcacaatcctgtactgtataattttggaatagaaatagtaaatgtgtcattcattgtatgctcttgcagggttatgtggtccgaatcagtggtgggaacgacaaacaaggtttccccatgaagcagggtgtcttgacccatggccgtgtccgcctgctactgagtaaggggcattcctgttacagaccaaggagaactggagaaagaaagagaaaatcagttcgtggttgcattgtggatgcaaatctgagcgttctcaacttggttattgtaaaaaaaggtgagggttacttgtgttcaattttcgttgaaattgatttaaagccagttgtccaaatgcttattcatttgcagtatccagacttacagaccaatggatttgtttctctaccatcaattcaacattaaacattctgatttaggattcttaaggaagttgttgaattaaaatctcttcaactgtttcttctaaggagagaaggatattcctggactgactgatactacagtgcctcgccgcctgggccccaaaagagctagcagaatccgcaaacttttcaatctctctaaagaagatgatgtccgccagtatgttgtaagaaagcccttaaataaagaaggtaggagggattatgcaattagggcttgcttaattttggtaatttgtctatcattttgtgtgcatatcagaagataaatatgtgcctttggaaggccttagcactattcacttggagtttttacagaaaatatttgggggtggccagcatacccagttggtgttaatccttgcttattatatccagtgccttgtagagtgcagtaatttcgttgaaaatctgcttgtgaatctttgatgtatacattatccaaaaataacctgactttaaggtcattgctatctggtttaggagtcacatgtttgagaattctgggactattagcaccacacactttgtaattttatatcagatgttctaaattattaatattttttaagagatgaggtcacactgttgcccaggctggtcttgaacccttgagctcaagtgatgctcccactttggcttcccaaagtgctgggattatgggcgtgagccaccacacccagcccaaaattattttttggcttacaaaagaagttgctttaaacttctgtttcatctgtaggatatggataacaatagttaagagttgtgagaattagaagttagagaccttactcagaatactagggagcaggtgttagggattttggagctttaccttttaagaacttttgtccatatagtagacaacatactcaccagtgaggacctcaaatagcaatatttctgtggtgaaatttggggatatttatgttaaataaacagcatgtcagtcaaggttgggttttcctgacaaaatttgaaagaatttctctggattttggttttcaggactggtgtgacttaacatacatggcatagatagtatacgtctgttaagtcagtaattacacttacttgagcacaggtactgttcaaggtgctgagaatacagtatagacaaattcctacccttatgaagctgatgttgcaaaaaaaaaaaaaaaagcaattgacaaataggtggcagttgcccataatcatagccctggaaataatccgtataatacaagcgtggtctggttgatgggtatttttagatggttcttatgtctatagtaggaggcttaacattttagcagaatctgaacatctgagcatttagcctaaggcagttaactcctacaaagtgtccatgaaacaaatacatctgaaaaggcctatgctccaatgcagatctgtaatccatgagcagttttggagtacagatcagattaatgaagatactttaacaacatgccacctaatctgatgttaatagttaacgttacccctgaattggcaggaattgtcaaaggaaatagagatgcaagcttacatcaaatttaaaagtatggcaggatgattatgtagataatgtgttaagtggttttcacagacaaattaactatgcacagttatatattaacataatgccactgtattttagtaaaactggaaggtagtaaaagttagcctggttatttttcaggacttggtttcccttatatttttataatatttaaccaatcttctagatttctgtggaggggcaaaaagcattttagaatagttctgatttgattttaaaggcctcattataggttttcatagatgctgttaagcttacgtttatttctgatgtttttaaagtagatgtggattctaacaaaacaaatattgaaatgattgagtcctttgaatttttaggtaagaaacctaggaccaaagcacccaagattcagcgtcttgttactccacgtgtcctgcagcacaaacggcggcgtattgctctgaagaagcagcgtaccaagaaaaataaagaagaggctgcagaatatgctaaacttttggccaagagaatgaaggttagtctaagatgatttgagggggtgggaggagttcgacctggctttggatttgcgtctttaacccagaaaggccttaactgtttgctgttttgttttgtttaggaggctaaggagaagcgccaggaacaaattgcgaagagacgcagactttcctctctgcgagcttctacttctaagtctgaatccagtcagaaataagattttttgagtaacaaataaataagatcagactctg

SEQ ID NO: 2 - DNA, 829 bp, Homo sapiens
cctcttttccgtggcgcctcggaggcgttcagctgcttcaagatgaagctgaacatctccttcccagccactggctgccagaaactcattgaagtggacgatgaacgcaaacttcgtactttctatgagaagcgtatggccacagaagttgctgctgacgctctgggtgaagaatggaagggttatgtggtccgaatcagtggtgggaacgacaaacaaggtttccccatgaagcagggtgtcttgacccatggccgtgtccgcctgctactgagtaaggggcattcctgttacagaccaaggagaactggagaaagaaagagaaaatcagttcgtggttgcattgtggatgcaaatctgagcgttctcaacttggttattgtaaaaaaaggagagaaggatattcctggactgactgatactacagtgcctcgccgcctgggccccaaaagagctagcagaatccgcaaacttttcaatctctctaaagaagatgatgtccgccagtatgttgtaagaaagcccttaaataaagaaggtaagaaacctaggaccaaagcacccaagattcagcgtcttgttactccacgtgtcctgcagcacaaacggcggcgtattgctctgaagaagcagcgtaccaagaaaaataaagaagaggctgcagaatatgctaaacttttggccaagagaatgaaggaggctaaggagaagcgccaggaacaaattgcgaagagacgcagactttcctctctgcgagcttctacttctaagtctgaatccagtcagaaataagattttttgagtaacaaataaataagatcagactctg

SEQ ID NO: 3 - protein, 249 aa, Homo sapiens
MKLNISFPATGCQKLIEVDDERKLRTFYEKRMATEVAADALGEEWKGYVVRISGGNDKQGFPMKQGVLTHGRVRLLLSKGHSCYRPRRTGERKRKSVRGCIVDANLSVLNLVIVKKGEKDIPGLTDTTVPRRLGPKRASRIRKLFNLSKEDDVRQYVVRKPLNKEGKKPRTKAPKIQRLVTPRVLQHKRRRIALKKQRTKKNKEEAAEYAKLLAKRMKEAKEKRQEQIAKRRRLSSLRASTSKSESSQK

SEQ ID NO: 4 - DNA, 8531 bp, Homo sapiens
gcagaagcgttccgtgcgtgcaagtgctgcgaaccacgtgggtcccgggcgcgtttcgggtgctggcggctgcagccggagttcaaacctaagcagctggaagggtaagaggtgttcgggatcctgagaggaaaaagaaaggagtgtagtcgcgggagtgggttaggtgaggagttagtgaagagatgagttcagctctaagaggcggcgaggaggatcggatgggaggaggggcagggagcgcctgtggacaagtgcagaccggtcggcgcacgtcccacgccacgtggactcgcctttgtcggtggccgcctttctcgtcaggccgcgacgacagggctgtgccttatttgtgtctgtcgagagcagtgccgggcacggagttggcgctctgtaagtgcttgctgaataaatgacgggtgtgtgtgtcattcattcagcaagcactgagtgcttactgttgtgtcaccgggaccgatgtggagggacatttttagggtgtatttctggcgctttagtcctgttttctcctggacaatttatgcttgccccgcaccccatcgtgcgattctccgcagtctttgggctttgtctctctctctttttttttttttggaagttgcagaatggtgataaatgattttctttgctcctattgactgctaggccctgtggctaggtaccatagagtctctacacaggactaagtcagcctggtgtgcaggggaggcagacacacaaacagaaaattggactacagtgctaagatgctgtaagaagaggttaactaaaggacaggaagatggggccaagagatggtgctactgtctactttagggatcgtctttcaaggcgaggggcctcctatctcaagctgtgatacagggtaggtcatgagcgcagtcttctgtaaaatgagggatctggacggaatagttactctctaggcttcctttcagtatgatgtcccgtatcagatattcctaatgtctgtagttctcccacaccccaccgtttattggctagtcaaaatattctttgcattgtaaagtgtagcataacattacaagaatggattaatgggttgggaatgctgggttttggccttcattctggtgctgtggtcctgtttcctcatttgtaaaatgaagggggttgtaaagatatattctctaaaacccctaatgctatggctgtggcattaagtcaaactattatttcttactgctttgtaccggacatcactttcacattagtaaaacttttcatcagtgtagtttctcccaagcttgattattgagaaagatagtgccttgtttttaacatcctgtatcctagttccaccgcagtgtttggtacacagtagatgtttaaaagaagcttattaaatcaaatggtggccaggctcatggggctcacgcctttaatcccagcactttgggaggctgaggtgggcagattacttgaggtcaggagttggagaccagcctggccaacatggtgaaaccccatctctagtaaaaaaaaatacagaaattagctgggtgtggtggcacgcacctgtagtcccagctatttaggaggttgaggtaggagaattgcttgcctgggaagtggaggttgcagtgagccaaggtcgcaccactgcactccatcctgggcgaaagagcaagactccataccaaaaaaaaaaaaaaaaatcaaatggggctgggtgtggtggctcatggccataatcccagcactttggtaggctgaggtgggcagatcacttgaggacaagagttcgagaccagcgtgggcaacatagcgaaaccccatctctaaaaaaaccccacaaaaaccaaaacaaatgaaaataaaataaatcaaatgggatagaaattatagtccttgcttttggatcctttcccacattgacatattagttcatagatcaggggactggggaggaattgggtttttaccagagggagacctggagctgacctgacagatttaaaccagctttatgggataatccgcttgagacggatgacgctgtaggcaagtgattcactgctgtttcattcctctacctgcctatccccagaaccatggccaactgtgagcgtaccttcattgcgatcaaaccagatggggtccagcggggtcttgtgggagagattatcaagcgttttgagcagaaaggattccgccttgttggtctgaaattcatgcaagtaagtggacttcattgttcccattttgattccttcatagtataggagaacactgtgattaacctgtttctccccgtctttcttatttaaagttctccacggtagagtgaacacaagtgtcttgagacctggaaactcctcagtgccctagcgtttggctacatcttggaacaaaccaagttatttaggattttccaaaatagtctctgtaataggaggtgcattgctacgggaaaagcagtgcagagttccacatatgactgcatctctgtaatttctgactgagagctgagcatgtttgagttcttggtcttgagcttcctgagcagataattttagagagatttttttccccctttgagacaggttgttttttagcaagaaagtctttccatcatcaattactagacactaataaatgcttattgtttagcatatattaggtatcataggagctggtggaggaaaactatggttttggttttggggaggttcttcagaaacagcagagagcagatgtacctgcaagctacttacaaagcaatcttttgataaatgcaaaggattgattgaccaataacccttcttcaaacaaattctagagataatccaaattctcggctgggtgaggcatagtggctcatacctgtaatcccagcactttgggaagccaaggcaggctggtcacttgaggtcaggagttcaagaccagcctggccaacattgtgaaaccccgtctctactaaaaatacaaaaattagctgggcatcagggtgggtgcctgtaatcccagctattcggcaggctgagacataagaattgcttgaaccagccaggtgcggtggctcatacctgtaatcctagcattttgggaggctgaggcgggcggatcacctgaggtcaggagttcgagacagcctgaccaatatgatgaaaccccgtctctactaaaaatacaaaaattagccaggcgtggtggcatgcgcctgtaatcccagctactcgggaggctgagacaggagaatcgcttgaactggggaggtggaggttgcagtgaccgagatcgcatcattgcactccagcctgtgcaacaagagcaaaactccatctccaaaaaaaaaaaaaaaaaagaattgcttttgaacccaggaggcggaggtttcagtgagccgatattgcaccacactgcagcctgggcaacagagggagggagactctgtctcaaaaaaaaaaaaagaattccaattctcaaatcaaataggggatagtatgttttctaaaagggaatcaattttggacccttctagtgcattttaaaatttagtttgacttgcaatttacttcctatgtgatattttagaaaaactgctttagggctggcacagtgactcgcctgtcatcccagcactttgggaggctccgtctccaaaaaaaaagaaaaaaaagaaaagaaaaactgctttaatgtaaaagagggtagactgatgttcatgctgagaggagaatatggtgtccaaggtcaaggagctggcatctaatgaaggttttcttgctgtatcatcccatgacagaatggaatagcaagagagtgcaagagtgagagagagcaagagggggctgatgttgattttataacaaacccactcctgagataagacattaatctgttcatgagggtgaagccctcatgacctaaacacctcccattaggcctcacctcccaacattgttgcatcgaggattaaatttccaacatacgaactttgggggacacattcagatcacagcagaaggtaacatgagatggatgtgttgttgatcaggcatttaccatatgctgggcacatatgttttctgatttagtcttcatgccagccttgagaagtacttactcctgttctcgtggttgttagtgatgagcaacctaaggcttggagaggttcagtgatgtgcctgatagcatacagtgatgccactggaattttaacttgagtctctctgacccctgagtgcagatactttctcctttacattgaatttcctattaaggatatatgggtttggtgaagaatggacggaaggatagctgccatgactctaaaggggcatttaaatctggtgtggaggcaggaagcaagggagaacttgaggttcccaacccccaagcccttaaaaaaagcccgtgatctgttttgcagactaaagataatcagttagggcctggcgtggtggctcacacctgtaatcccagcattttgggaggtcaaggtgggtggatcatctgaggtcaggagatggagaccagcctggccaacgtggtgaaaccccgtctctactaaaaatacaaaatttagctgggtgtgatggcgcatgcctgtagtcccagctaccagggaggctgaggcgagaggatggcttgaactcgggaggtggaggttgcagtgagcggagatcgagccactgcactccagcctgggtaacagagcaagaccctgtctcaaaaaaagataattagccgggtgtagtggtgcacacctgtagtcctagctactgagaatctgaggcaagaggattgcttgagcctgggagtttgaggccgcagtgagctatgatcatgccattgcactccagcctgggtgacagaaagaccctgtctcaaaacaaacaacaaaaccaacaaaaagataattggaaccaattaatagaattaaaaaagtattctgataagggaaggtcacctcagggtctgttagacttccacaacaaaagtcttccatttggataggaaaggacagggcgagggttggatccctaccacggagtgattatatggccatgtaattttactttggcaagtgatgataactggtggctgaatgatggggcttctgggcagccagtctccacatctgtgtgtgtctctgatgtgtgtgatatgcgtcatacagtgcagtaccacagagctggaaaaaccatgggaccctgttagattttaacaagcgtatatgcagtacaagagagaacacagccaaactgagttgaaactgctaagcagatggtttgttctctagctaacttgattcgttcccacatattagccttgcagtatgagaggttctaattcatttttcagcaaaattatttattactagatgtcatgcacttttgggtgatttgcagatacctttggtaatcacatggtcatcactgctgggcttttatcctctgaataaacgtgaagcatccctgggtaatggcctggtttagtttgctgctcattaaatgccaagacttgcttcctggtgtccttctaacattttatttggggtttgctctgggaatggaaatttattgtattgttgattttcctgtctaaaatgagatagttgagggccaggcatggtgggtcacgcctgtaagcccagcactttgggaggcggaggctggaggatcacttgagtccaggagtttgagaccagcctgggcaacatagtgagaccctatctctacaaataattttttaaaattagccaggtatggctgggcgcggtggcttacgcccgtagtcccggccctttgggaggccaaggcaggcagatcacctgcggtcacctgagaccagcctggccaacatggcgaaaccctgtttctactaaaaatacaaaaattggccaggcatgatggccgccacctgtaatcccagctacttgggaggctgaggcaggagaatcacttgaacccaggaggtggaggttgcagtaagtcaggatcacgccactgcactccagcctgggtgacagagggagactctgtctcaaaataaataaataaataaataaaattagccaagtctggtagtgtgtgtctacagtcctacctactcgggagtctgaggcaggaggatcacttgagcccaggagtttgatttttttttaaatgagatagctggatttaattttaatgttctgtggttttattatcagtcagtgccaatagggagacaactgatataaaattggagaagtacatttagtcctgtcactgctcccttccagtgtggagaatgaattgggttataacagaatcagtgagcccaaccgctcatgttttacatagcagggtggatgaggggaaattaaatggattatatgtccttagatggtttgggggttattctcattctctgtcctgttgaataggcttccgaagatcttctcaaggaacactacgttgacctgaaggaccgtccattctttgccggcctggtgaaatacatgcactcagggccggtagttgccatggtgagtgtgcctgtgtgggatactccaagtatgcattgcttgtcatctgtgctaggctctcttctagacactggggatacagccatgaatgagaccaaatagatacctgttttcatataccagctaatggttagtgataggcttcagaaatgaaatatgcccagaaatgaataggagtagcaaatgatcactttagattgggtgatcaaagaacgcaatttttaagctgtgatctgaatgacaagaagcagcgatctgaatgacaagaagcagccagccatgtacagaatcacagtgaaaaacatttccagccaagcaaacagcatgtgcaaaggtcccagggtgggaatgagcttggctttttgaagtacgggaggagagttgaaatgcccgaagcctgatgggtgggagggaagttgggaggtgaagagttcagaggcatgggcaggggccttctctcttaggggtgtgttgaccatatacttcctgagtgctgggaagctatgggacacttcacacttggcatttctttttttttttttttttgagacggagtcttgctctgttgcccaggctggagtgcagtggtgccatctcgtctcaccgcaagctccacctcccgggttcacgccattctcctgccttagcctcctgagtagctgggactacaggcgcccgccaccacgcccagctaatcttttcgtatttttagtagagacagggtttcaccatgttagccaggatggtctcgatctcctgacctcgtgatccactcccctcagcctcccaaagggctgggattacaggcgtgagccactgcgccctgcccacacttggcattttaaggaggctgctctggttgctgtatggagaatacattgtagaaaggcaagcatgagggcagggagatcaggatcctcagtgctggaataagtggatatactctataaacatttgttcagtgaatgaatataatcttaaggggcctttttatttttccttttttgaattaatagttgccagattttctgctgtgattggttttcttctttgaccatatcttcttctgtccttggaggtctgggaggggctgaatgtggtgaagacgggccgagtcatgctcggggagaccaaccctgcagactccaagcctgggaccatccgtggagacttctgcatacaagttggcaggtgagattttggtatttttcccccttttccaaaatctgatttagttgccacaaggatttgggtttccgaggctggaggtagacatgataccatatgcaggttgattttatcggagtttattgttttcctccctcttaagttggcgtttggctttggttgttaacatcacttagtcgtacctctgttacacgaagtgtagaacctggtacaacacactttgctggccagtctttcctgaaggctctcactgtttgcaccagcgtggccgggagggctggatgtggggagggcatttttcagccttaagtgaatggccattgtaggctttttcttttaacatggtctaatgtccatggagcttcagcttttatgctgctgtggctgtagatttctggcaatgggcgcattttaatccttctggtcttggtcatgtgactatctctttctccacccaggaacattatacatggcagtgattctgtggagagtgcagagaaggagatcggcttgtggtttcaccctgaggaactggtagattacacgagctgtgctcagaactggatctatgaatgacaggagggcagaccacattgcttttcacatccatttcccctccttcccatgggcagaggaccaggctgtaggaaatctagttatttacaggaacttcatcataatttggagggaagctcttggagctgtgagttctccctgtacagtgttaccatccccgaccatctgattaaaatgcttcctcccagcataggattcattgagttggttacttcatattgttgcattgcttttttttccttct

SEQ ID NO: 5 - DNA, 1031 bp, Homo sapiens
gcagaagcgttccgtgcgtgcaagtgctgcgaaccacgtgggtcccgggcgcgtttcgggtgctggcggctgcagccggagttcaaacctaagcagctggaagggccctgtggctaggtaccatagagtctctacacaggactaagtcagcctggtgtgcaggggaggcagacacacaaacagaaaattggactacagtgctaagatgctgtaagaagaggttaactaaaggacaggaagatggggccaagagatggtgctactgtctactttagggatcgtctttcaaggcgaggggcctcctatctcaagctgtgatacaggaaccatggccaactgtgagcgtaccttcattgcgatcaaaccagatggggtccagcggggtcttgtgggagagattatcaagcgttttgagcagaaaggattccgccttgttggtctgaaattcatgcaagcttccgaagatcttctcaaggaacactacgttgacctgaaggaccgtccattctttgccggcctggtgaaatacatgcactcagggccggtagttgccatggtctgggaggggctgaatgtggtgaagacgggccgagtcatgctcggggagaccaaccctgcagactccaagcctgggaccatccgtggagacttctgcatacaagttggcaggaacattatacatggcagtgattctgtggagagtgcagagaaggagatcggcttgtggtttcaccctgaggaactggtagattacacgagctgtgctcagaactggatctatgaatgacaggagggcagaccacattgcttttcacatccatttcccctccttcccatgggcagaggaccaggctgtaggaaatctagttatttacaggaacttcatcataatttggagggaagctcttggagctgtgagttctccctgtacagtgttaccatccccgaccatctgattaaaatgcttcctcccagcataggattcattgagttggttacttcatattgttgcattgcttttttttccttct

SEQ ID NO: 6 - protein, 177 aa, Homo sapiens
MVLLSTLGIVFQGEGPPISSCDTGTMANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGSDSVESAEKEIGLWFHPEELVDYTSCAQNWIYE

SEQ ID NO: 7 - DNA, 36401 bp, Homo sapiens
gggagaaacgttctcactcgctctctgctcgctgcgggcgctccccgccctctgctgccagaaccttggggatgtgcctagacccggcgcagcacacgtccgggccaaccgcgagcagaacaaacctttggcgggcggccaggaggctccctcccagccaccgcccccctccagcgcctttttttccccccatacaatacaagatcttccttcctcagttcccttaaagcacagcccagggaaacctcctcacagttttcatccagccacgggccagcatgtctgggggcaaatacgtagactcggaggtaggcatccgtgggggggcgccggctcgggcgtgcggggagtgtccgcttctgctatctgcctctccaaatatcccgactgctgccctggccccagccctctctccacttcggagcactcctctggcgttggcaccgctgaggaatgggcctgggcggggaggtgaagagaagccaggaatgttttatgttttcctaatggagagggggcctagggagcccctgagctaggaggacacggaaaaggggattggggtcctgagattgggtctgttgggcccaggacgcgttttctggatgggtctaggatgctcccttgtcgcgggacccccgcggtccggccctgcctgctgggggttcgaagaggtggagtgcagggtggaggtgttatttacccgagtcctggggacagtccccgggactctccgccaggcgcccagaccggcaggtcccgcaggcggcgcgcggtgtgtttgcactttccaaagttcttgaaccatctcaagaactccttctgcatcttggcgtctggcaggggtgttccgagagaggtagacctcccctccccaaactgccaccatcacttccaacgccctccacgcgctggagctctgcccgggtgtggaaacctcgtcttccaacacgtagctgcccttcagccacccgcccgcagcctgggagtgccctgagggtgggtcgggggagctgcgcaggtgagactgagttctaggacatttagggggtctggtgcctggctccgccaaaaatggggactttcgggattgtgatcatcacggcggattgagcagggagagccgtggagggacaagagagggccgaggcagggtggggggcgcgggcaggtgcgagggggatgcggccaagaagcagcgataaagggaacattccacgggtcgggcggctgctgttggatcttagataaagctggaagggattaccggggcaggggtaatagggaccggggacgggaacgcgaaacaggtgaagcgctcagggcgagagcgactcggcttagggagtccgggagaagcctgcggctgccccctcgccgccgaggtcctgcgggtcctgcgggtcctgcgtgctgagccggggcgtgcgcgggcgggggccttcggaccgcgcggcggggcctgccctgacccctggcggcgggcgggggaggcaggcgcgccctgcagagtacagaggggtgtggtgtcctctgcgagatcctcttaaaaagctggctacgcgcaggcggtttctgtgcacggagccgtagctgtcggagcggttagttcgatttcgagctcgaggtttcccccgccgccaggctgacttctcatcgcttgtttttctttttgcatttttcctcccaccgccgttgccgccctccccgtcctggccgtccgccctccgccctctgcagggacatctctacaccgttcccatccgggaacagggcaacatctacaagcccaacaacaaggccatggcagacgagctgagcgagaagcaagtgtacgacgcgcacaccaaggagatcgacctggtcaaccgcgaccctaaacacctcaacgatgacgtggtcaaggtaagccaaggcgaccaacagggaagggctgggacagctctcctctggcagttagcccgtgcatccttctttagcattgccgtgtacgcacaccccaccccgccccctacacgcgcacacacacacacacacagagttttgtgggtttgatgtgtgggagctcccgcagtcggcagaaacgttacatctcccttcccccatctccccccaatagttagttcagctgaaattcagctaaagtgagttttgtagaagttcctataactacacttttatcctagcaaatgagcctattgacctcagcaacagacggcccatactccttgggacggtgagatggttcctatccattcccaggttgaaagtctagtgacaggtccccactgcacgtggcattaagacagtcagataattgtgtcaggtcttgtgctgaggatgagtcagaatacaagatgggcatgttcccccaactaaaacgatgggaagtgattttcttaaaaatactacagtggatggaaatgcctaggactaaagacaaagaaaatacgtacttattcatatacatatgaaagttactttaactagactaacaagtcacttgtgcacaactaagcaaatttacaaaaccaaaaacaatgtatgcctcttggtttcttctatctatggacacctgcacttagatgtggaaagctgcttctttagtagctacctgggtcagcctgccctgagctaatggcacattcaggttggagttccttttcatactttcaggatgtgcttggtgagattaaaaataattggactgggttattggccagacttagatctgactcagtggtcagttttaaattatcattgttattagattttgacccttttagccaatctagtgggaggaatttattgcctaaacacatctggattgggatatcatgggctagagccatccttggcaaagggttttctctgagaaatggagggctaaggaaaaatcctggctcagggactgcagtgtgaagatctactcctatacaacccccagcaatcaatgaggcggatgagcaatttccacccaccacgcctgctatctatggatgggaggagctatagttcacaaaccgtttacattcatgaataatatatttcaaaaggggaaacagtttaatctgtaactggaagggaaaaaaaaactgtcagaattgactcccttggcttcctggagtaggaaaaaggaaaattggagcatttgcagctttttttgactagctggattatggaatatttaaaagcaacagcaacaaaagtaccttataaactagaaaatagaattgctaaaaaactatttactaaaaacattaccttaaagggagaggatatttgtgttttcccccacccccacccttctcatgtggctttgaacaagaaggagagttgccaggaaaagaggcagatttcagagagggctggcttcactggatcctccctgttgttccactgcactgtgagtgagattccctggagcaagcgaatctcccgggatgagtcagagaggccaacagtgtggatgtgggtctccacacatagcatgactaagttgagaaagaaaggccccactgggaaaagagacttcaacacagatggaaaaaaaacataacaggcttggaggaaatagcagtttacaaaacagcatttcaaagagcaagtgtggggatcctcaaattaaagaaattaaaagaaaaagctagagcaagctcctgctagcctaaagaaaccaaaccctgactacttgctcatagaactgtgagcaaaacaagacagtcaaaccaaaaaatccacctagaaaagaatttggcagtctcactcagatgcctggcctagaggggacttcagagaatgccctacagagagacaccaagactacaaatgcaaattctgcccaaagagtgcctggccgatgaacagggtcctatctacatcttatggagactcctattttataaatatgtatcctcaagtccaagcacaaacaaaataacagaaacagggatgattctctcccagtttccatgacagtaaataataaatttccctaaattttactttcaacaacatagactttttttatttttatttttatttatttatttatttattttttgagacggagtctcactctgtcacccaggctggagtgcagtggcatgatctgggatcactgcaacctccacctcccaggttcaagcaattcttctgtctcagcctcctgagtagctgggactacaagtgcacgccaccatgccgggttaatatttgtatttttagtggagacggggtttcaccatgttggccaggctggtcttgaactcctgacctcaagtgatccactggtcttggcctcccaaagtgttgggattacagatgtgagccactacacctggccaacaacacagacttcttaaaaaaatcatgacaataattttgggtgcttcttaaaagcacccaaagctttactgctaatgcatggtagcttaaaacttcacataataagaaagaaccagtggccaatggaatctactgttaaaggtacccaatcaagtaaggaaaagttggtcctaaaagcaagcagccctgtaaaagctgctctgtccaatatggtaatcactagccatttgtgtttccatttaaatttcaagtaattaatatcaagtaaaatttaaaattcagttccttagtcacactagccacgttgtgagtgtgcaacaggtaaagctagtggcacagacatagaacatttccatcagcacagaaatctctattggacagtgccagattagggtgttctctgcattgtaaaagcatccccttgccaagttaaagaaaacaacaacaaaactctagagaagaaatgaaaccccagtttcatttctggagaggaaagaaaactcatgtgtggcatgagtttatattcaagaaggtgcagcattattacctattttactagtaataatgacacacattatagtatacaatccagttccaataaaattaatttctcatcttactaaaagcttgctgctccacattatgagacaatttacccaaatatagacatttacccaaaaatattaagtagcttgtgaatactttttaaaatttcctttaattaaagtggtcacaaactcaaacccttcattctccctctgagatttctgtgtcatcttttgttcacattgttattcacatgtttattatgtacttattttgattttctagataaataaaatggcttcaaatctataattctgataaaattagccatcaattaatttatttattaaacccatgcaatatgctagattagatgctttgctatgtaattcctacaataaatcctagcaatcacaaagattacagttagtgagacgacatgcacacaggtaaaaagtgtttttaaaaaatacatacatacaaccaaaacagtaagtcactgctacatggaaactgattggtcctttttcctttttttttttttgccttgactgccaggaagcagtttcaaatctatagctggattttaagtttcattaattcatgttcccacatatggttctgtattttcacttcccccttttaactgacatactgtcttatgtgatctctactgtaagccttctcatcattttggaaacagaccaaatataatatatatgataaggaatcaaaagtaaatacagtagtgttgaatattgcataacaaaaaggtttttaaatagggaatggtatcaatatgaagtgttagggagacccagccatgaaaaggatagcagggtcagagaaggaggatgtattgcagctggtttaatggagaatggtatgaaggaggtgcagtttgaattgggtcatggaggacagatggattgcaaatagctggggcaaaagcacaggaaggcattctaaacgagccaggcatggagacaagaatgtctcccacaagggagttgtagtagctcaatcagactgggatttgagatttcatgtggcagagtggtaggtgataaaggtgaaaagactgatcatagtaaaatgcggagtctgtaaatccagcactcatgataagtttggacatcatgtcaacagtggacagccataaatgactgcaagcatcggtgtggtataatgaaggtgacgtttttgtaaaatgactctggtgaaggtacagaaggtaatgaaaagtagccagtctagttgagcagaaaagagttcagatgtaattgcatcatggtccagatgtgaaatgaagacaatgcgaagtggcattgtggatcgaaacatacatgcacaaaatgacagaattttagaatttgaagggatcatcatggttaccaggctggcctccaattcctcttttgtaatattaatagaaattaagggctaacaagtttaaaatgttatccatctttttacatagttactgcccaaagtgaatattttgaaatgtatcattaaagaagaatagataagattatgtgattcaccatggactattgtcatgagaggaaaaatgtgtttagatgattctgttagcactgagacaaatcaggatatctgaaaggaggtctttgttgaaaaacagaaatatgcattcataacttgcttttctaaaattggaatgtaatgattcttaaatatgcacagacacaaatttttctttaacagtcaagaaaatgcacgcaggtgataatcagatcagttttggttatagtacaaaggtttaatgcctccgtgatccctttcaacttgaaagcattctagagcaattggtgattaatatcagtataacagtcatttataaaattattatttatttgatatacatctaatcaaagcataagatttatttttattattattattatactttaagttttagggtacatgtgcacaatgtgcaggttagttacatatgtatacatgtgccatgctggtgcgctgcacccactaactcgttgtctagcattaggtttaaaagatcagattgtctcggcaccatgttaatatctttttctgttggcattagtattagttttgcttgtgtatttgtttaggagatagcttcacaagttggtgattgatattctaccatgtatgaagtcatgcgtggaattcagaatccccagcttgtaaaattgcattatgatcatctttagtgggaaattgttctcagaatactgagcaaaggatgataccaaaatggcagctattattcattcttaagcatatgaaatgctttcaggttcaacccaaaattacatacattttaaatgcttactaaaagagtcttttccctcctccatctattaactgcaatcaaaaaacttcggttttaactgaacatgatttcatattatttattaaaatttaaggcaaggtgcaccaagtacccttgaattatgaaaagcttcatgatgtgggatattctttcagttaacggcagggttggctacacttttaaggggttcaaagtaggaacagctgcaatagtgagctgcatctggaaagtccagtaatttgaaaaaccacctgtttatgtatcctgcccactcaagtccataaaataacagacactttcatattccaaatgaaactgctttttagtttgccctacttttaaacataactctttgtgatggaatgaccagaaacagctggtctctaagaggacagggctatgtgcgctcacctgcggggttggaccttccataatccccctggctgtggggaaagttgagggctgctgtctttatacaaagatggtttattccaagatacacacactcttcttccacaccctggagaccttgcatatttagtatcttctttaccataatctgaggccctagagaaaaagatttgcaaactatacttgttttaaaacaactttctaaaaaagacactctcagcccctagaaattatgcctaacacatagatgctcagaggcaacctgttgtagtgcaagaggattgtgccaagattagaaaacaaatatttgcaacttttgtaactgtcttctctaaaacttgaatgtggtgattctaaagtaaagaccgacacaaaattctttttctttagcagtcaggaaaaggcatgcatgaagtaatcagatcaggtgtggtttcagcataatggcctaatgctttcatgatctctttcaactggaaagcgttctagtcccactggacaccaaggaggaagaagggacggaaaatattaggcccataggtttatcttcctcagtagtccacgagatttgagcttatatgtagggagcaaaattgtttgtctaaaagcagttaataaatgccccaaaaaggctgggcgcagtgactcactcctgtaatcccagcactttgggagctcaagattggtggatcatgaggttaggagagcaagatcatcctggccaacacggtgaaaccccatctctatgaaaaatacaaaaattagctgggtgtggtagcgcgtgtttaatcccagctactggggaagctgaggcaggagaatggcttgaacccaggaggccaagattgcagtgagccaagattgcgccactgcactccagcctggtgacacagcgagactccgtctcaaaaaataaaataaataaaataaaataaaataaaataaaataaaataaaataaaataaaataaaataaaataaaaataaaatgaacgccccaaaaatattttgggcaaactattttgtgtttcttttctttatttatttatttcttttgagacaaaatcttgctctgttgccccggctggagtgcaatggcacaatcttggctcactgtatcctcaacctcctgggctcaagcaactcctgagtaactgggaccacagggatgtgccacaattcccggctaattgttttagccaggatataaatgctgcctacatagagtttgtagctatctccttgactttctttatgcagattccttcacaaacttttgatggattcctttaccaaattctactgtctgttaaaatcttctatctttatatctttagtccaaacaacacgtcatttataaaccttaaaattgtttctgggcaaataaacaaggcaaaataggaatatatatttttaggcaatttacttctgttttggtctcataaaaaattgtaattaaattgtagaaaatatttcaattcctctttaatatcctctcctcacatactggctctcaacttctaatcctcctattgaaacattgattgggaggccaaggcaggcggatcaactgaggtcaggagtttgagaccagcctggccaacatggtgaaaccctgtctctactaaaaatacaaaagattagctgggcatggtggcatgcacctgtagtcccagctactttggtggctgaggcacgagaatcgctttaacccgggaggcagaagttacagtgtgccaagatcaagccactgaactccagcctgggcgacagagtgagactccatcacaaaaaaataaaaataaaaattgaaatttgcagcctttttaaaaccccatagcctctttataaacccaaaagcactatcaaatttggcgaggtgtcaaaagaatcagaggaatgtttacaaatacagatgcctgggcccacctcagatatatatatatatatatatatatatatatatattttttttttttttttttttttgagacgatgtcttgctctgtcacccaggctggagtgcagtggcatgatctcagctcactgcaagctccgtctcccgggttcacgccattctcctgcctcagcctcccaagtagctgggactacaggcgcccgccaccacggctggctaattttttctattttttagtagagacagggtgtcaccgtgttagccaggatggtctcaatctcctgaccttgtgatccgctcgcctcggcctctcaaagtgctgggattacaggcgtgagccactgcacccggcccagatatattaaattagaatatctagaggtggagcctgagtatctgtatttttcagagtttcaaatgatcgttcttcaaatgattacactgtgaagtcagatttagaaatgactgtacccaaggttggctaaaagatacacaccctggttgattctacctgaagagagcaaataagatacacagcaaagttgtagatgttttccctgccagtagaatacttgcgggttaggccatttaaaaccctgccagagagttttgaaacactgtggagggctcccaaatcaacttgctcaatggttctccatcccttcaggctacttgggcttaaagccaactgcaagcttagagcctcagagtgacctaggaatggggtgaccatatattctaggttgtctcatacagactagccagcactactcagccgcaagtaatagcatccaggcatgctcagaagtgtcccatttggaggaaaaaaacaatattgtcacaaatgaattggcaatggcctgtctctgattcttatacctggaatatactggaagtccctactcatgctattttctagcagaataggcaaaatttctacattccaggcatgtcaggcctttccctgattcctttctctaatgtcactcgtctgctgtcttttatcacagccattaaactgcaccctaacttaaagaggatcccttatgttccaatctactcatccctcagatctttctttctctgaaacacagggttaatgagactgacatccttccatcacatattttctcagctactcagtaaaagatgtaaatgtttaaaatagtttaaactatttttcagttagtccaggaaacataaaatggcatgcttgcacataaaccattgtttagggtgggggaagtgtttttaattttgccttaaaggaaatctgcatgatccacaggctatgcaactaccaagggaattagttggtagaacagaattacacctgcacagaatacaaatttcctgcctttcatgggaactatgttgatgtttcagatatgaaatacatcttgttttctttattgaacctcgagaagatgtctcttgttggtcattatttcatggcaggggaagtacatattcctaaagacacaaccgagtttccctttaaccatcattagttgggctggccattaagaaccagacgcttttattttcaaagagacttaagttttgatgttgtacatatgtgcctaatattctatctcatagcaatttaaaggtgacgttttaaaaagctgcattcagtgtataaacttctcctgatcccagcaaggatgttgtgatgattttatttaaaaaggtaagttgtgtctagatatggcagtgggtcatctcatgcatggtgcagatgtcaaacacaattacattttcttatttgcaatgactaaaaaaagaagctgagcccaagcagtgagaaagtaggagattgggaggacaagaagcaaaggaaaaaagtaacatgagcaccgttctccctgtcctgccacttgctccattatggactgggctgcgatatctcatatcccagctccacaactcccaacaaccatttatgtgcatggtgcttccatgtgtgatgacccaatcaggctcaggtgtggactgagtagttaaattataacccttgtctctgaagagtttagggcttagtggggaaacagacatgtaaacaaacctgagtgaggtcatgtaatcaaaggacaggccacagtcaaccacaaagaagagagttctcagcagtctccaaagccgaacatatgtttaccaggaacagggtcccagcagagggagcaacaggagcaaccagagccttgaggggtcgtggcctgttctgggcaccagcagtggatcaatgtggccagagccagggatactagcagaagccagagcagcagggccttccttgtccagcaaaggcatttgtctctttgtaggccacagcgacccacagagggctttttaggccagaaaaaagccattaaggccgggcgcggtggctcacgcctgtaatcccagcactttgggaggccgaggcgggtggatcacgaggtcaggagatcgagaccatcctggctaacaaggtgaaaccccgtctctactaaaaatacaaaaaattagccgggcgcggtggcgggcgcctgtagtcccagctactcgggaggctgaggcaggagaatggcgtgaacccaggaagcggagcttgcagtgagccgagattgcgccattgcagtccgcagtccggcctgggcaacagagcgagactccgtctcaaaaaaaaaaaaaaaaaaaaaaagccattaaaaagggagtcatgtctcttgttggtcattatttcatggcaggggaactacatattcttaaagacacaaccatttcctcttaatcctcattagctgtgctggcaattaaaaaaccaaaagtttttactttcaagaagatttaaataacttctgagggtgtacatatgtgcttaatattctgtctcacagtaatttaaaagtgaagttttgaaaagctgcatcctgcgcttgtcagaaccatgtctgatgagatatcccctttaaagggctctcggtgcaatggggcaaatcaagggggtttgtgcaagtgggagtgagacaggagatggggtgcttcttccagcactccctataggctgactgagtgacaaagatcattttactgacacctccaatggccctatgagatgggtactattattattatcaccatcatattccttttgcagataaggaaactcaggcttagcagattgccagaacaacacaggcaggaagtggtagagtcagggtttgaacccaggtagtgaaactccaaagcccggattcttaaccactgtcctccagtgcctctctgtaataagtcatgatcccagaagccattggtgtggccacaatatggaaagagatgacagtgtcctcacactgggtgagcagcttatggtgattccagacatgatctctgttgggagtgacaggtctgagcttctaggatcagaccctagatcttggcaagtggtttgaggaaagagaaggaccaatgtaaaaccccaggcttcaaggaatgtggatgctgggcagggaggattaagccccaaagaccagaaatggggtacacagggcaggtgtggccagagtagaactagagtagaacttccagtgactagaaatagaaccagacacgttgcagtggtggataaggtagaatcgcttaagtctttaaagtgcccctgatcacccaagttggccagagaccctggggtggggctgattctgtctggatatacggggaggggtaagcatgaggaaaggaagcaggtcctgacaggtactttgcactaaacagctccttataaggttctcaatttgcctgctcaatttctacagacatttgtgggaccacaccagtacattgtaaaagcaggaaacaattgagaaaaacctgagttttatgttggtaggagaaatgcctatggaatatggcaaatcgtttctctgagacttcctccctagtaattacatatttgttctcaaaaacaaatgccagaaggaagaagcagatttaatagtgcattttacaaggcaccattaatctctaagaagaacaattaaaatgtctcagcaatcatggttcactgtatatcttttctatcttcttagaagtaatatatggctggaaatgggcataccaaaatatgtcaaggaagtggaattgcgttcattagatttcaccactaattattttagttagcttcacagatctctcttccttgcttgttcttgagagcgaggctttttagtaggaagagaaattgtctaaaacgattaataaccacaaattcaccaaactattttgggtaagtccctctatttctctaggtctaaagctaggaataagagtcattctcatataatgtactgtcccagaaagggcattatattagtctgttttcacgctgctgataaagacatatccgggattgggtgatgtatttaaaaaaagaggtttaatggactcacagttccacatgcctggggaggcttcacaatcatggaggaaggtgaaaggcacatcttacatggtggcagacaagacagaattgagagccaatcaaaaggggaaaccccttataaaagcatcagatttcgtgggacttatcactaccacaagaacagtatgggggaaccgccaccatgattcaattatctcccacaaaatgggaaaattatgggaactacaattcaagatgagatttgggtggggacacagccaaaccatatcaggcattcaaccaatatttgggaagcaccagccctgcaccaggcacggagcacgtcatgagtcctgccgtaccacagcctgcctgacagacctcagtcatcctctggagcttgcctctgacatctggacctcctcagaatcagcatctcttctccttgcccccgccatcctttgtttttatctctgctgtggcattcatcaaagccttccaactatcctgcgtcactgtccttcagtgtcctctctcctctcccttccttctcaccccactttgtgcctgtatccttcaagcagagcaatggcaccctcacttctgtggctgcccagtgccccatgcagagtcagacatcagaaaatagatgctgaattcagttgacactctgaaattctttttaaagtaagttaatgtgtgctttgaatgaaaagacactgggattacattattgagtgtctttcttcctttgccacttttgtccctattggccatatttgaaaatcttgttggaaaaaaaaattcaagaacttaataaataaattcaaaaacatttagtctatttacttaggtgaagagaaaactcattctaatatgtgtgtatatttaaaatatttgttatttagactttttttttaagtctccaggttgaggaggacacaaatatatcctcctaaaccttccagtaagcaagctgtggcatccagatgatctcctgggtcatgggggataaggctaatctcctaggtgtctggcagacaggacaggcaaattcccagaatgccaaaatataccatctgctgctgtttggcattgcccttaagtccagagtgtggaggctgggggtgggtctctggctacaggagaagtcccctggcaagggaggggtgaaaggagtgcctgttgaaccccccatctatccccgcactatggcaagattgagaggaatgactagatcagggaatggcccgaaagaaaaatccaaaacctcccaaccctggacaaggccacagctttgagaaaccgaagcctctgcttccttctctttggctttactgcttctagatgcaaatacacagagctctgagattttgtgtgctgggaggtgataactgttaaccctctattccaatagcacagaaatttctctttgcctcagaagtggtttctcatagatctcagatctcttttcaggaaaaagaaaaacaacaacaataacaacacattaatgactctgaaagagtcagacaccattaattccattattggtgtctgtgccaagtgaaatgaacgtcagctcttttcccagatatgtttccttcttttgcctcctataataagagatgattttactgtaataatataagactcatcaatttgactccaaatagctttcctatcaacaggctaagtgtaaaataccaggatcattattcagttgagaatagatagaactaggaagtagccatcaaaaaagaatgatgaggtgcattgtggatttggggtgtaacttggtatctaacatacagccagaatcacagtcatagcacacttaatattttatcagaaacttgcgtgaacaagttaagaggactctcaacttaaaaatgacaccaattgcaatgatcttgttaacatttgtgatgaaaataatagcaaagtgacttagacaaattacaatagcccataaaaataagataaagtttaacacaaagtaagatgatgttaaaagacttgaaataaaacagatatgttaagtaggcaacacataggtaagcatataaaaacaagaagataccaggatagagctgtcatttttgtgggagcctgtgatgtggaaaaccaagatgcctggtgagtataatggatatggaaaccccccttgtaataattccacagttccaaggggccaaggtctccaggttgagtcactattgtaaacacacccatagatgaatccacatgccatacctccttgagtaagtggggactcaaactaggtctgtcaattgttccagaaaattaagcatctaaataatttaatgataatttaaaagaagcacaatgaaatatttcaaggaatgtcacatacaagattctgtacctcttctgctttggttagactcattcagaataggttcctgctttgatcttaagagggaggtagagattctggagaagccctagggaagagcaaaaggaaaggaataaggagccaagaggaaacccagggtaaggctgaggagggactgtttcgtgtaggtgatttattggaagggttggaaggaaacatggaatgacaattacctttggttattgtcaggttagtatgagacttacaagaaaagcactgctcagacgcaattaccattcaagataagaaataataggaaaggctagcacacttagctttttatttaaaaaagtgttaggtaggctgagcacggtggctcactcctgtaatcccagcactttgggaggccaaggtggatagatgacttgagcccagaagcttgagaccagcctggacaacatggtgaaacctcatgtctacaaaaaaatacaaaaattagccaggcatgatggcatgcacctgtagtctcagctacttggggggccaagaggtgggaagattgcttgagcccaggaagtcgaggctgcagtgagccatgattgtgccactgcatgacagcctgggcaaccgagtgagagcctgcctcaaaaaaaaaaaaaaaaaaagtgttaggtgacatgagagaagatcttccaagtaataagagtggctaatcccaggaatgtgtcaccagaggttattttgtaatagtcgtgtgttaaattccttatttgtctatataacttctcaaatccttctgcctctacagttatagtttaactggcgcataacagccttcacacacagcctcataattaaacatagacatacatatgaacactttcccctatgccagcaggatacttggtttgtttaggggcaaagaggaattgatgtggcgttgtttcaatcagtggttgaaaatgcaagtggtaaacattgaaaaatagaacactgcaaaaggcatgcattgtatataccaaaaggtcagcatgaagcattatctgtatggcaagcctgcccatccactccctcctacacgttgcatattcacacagttttgcagcttgtataaacccctattgtgatagaaactcatgaaagagtgtggtctctgcgaaagctggctgttctgtgaatttagaccagtggttcttcaccctggctgcaaatcatctggggaacatttaaaaacactgttttaaacaccccaaccctagaaattctgatttaattggtctgtggtggggcccagaactctgtattctttttttaaggctctcaggtgctgctaatgtatagctaaaattgggtctggtttagactctcagaatttcttaataattaaacactttatcatgacaagactttcaggaccttaaaggccacagtggggtagttatcatttcactaggtcctcatctggggaggtccttggcatttttactggaatatatttgtcactcaaatttctattacaaaaaattctttcttgcacactgctttagcaactacatgagatatactttgtacatagcacaaatctcatatcacttatgtaatccagctctgtggttccttcctttcctttgcctgtttatttttaattcttcccaagaggaagcttagccagttagaacaccagagtatcatccccctcccccttttcccacctgagttcatggcttagacatactaggaatgaagctgacaacatgcactagtttttttcgaaattatgcagcaaaattcccaaagtgcgagtggccacagagatcttcacagggcccagggacaggcagacatcattctttctccagttcctggcacagaaaagagaccttaggttactgagaagataccagtccctcctcagagcagacaaggaaactgagcctcagaatgaaagactgaatttcagtcctttcttgaacatggacctccagggttatattgggccttggaaaaggcacttacactctggactgtagtttcttcatctataaaatcaagaggcagaaacagacaatctctaagttgcctttatttataaaattccgagattctagttgaccagtattcatacaagagttgaagcctgtaagagtgcagaaagcccacacaaagagacagtggaagacctctcatcagtagtatttttattaccctcttcctaggttttaccagtcaacatcctcactgttaatatacagaccgtggtatttaattaaatcatctttgaaatactgagctatcaacagatggcatgctgaatgcaaaaggaccacaaataaatatttggtactgaagaagatcaagagttggagttcatttcccattctgatctgggctcagaactctgtggtcttccctctaatcatccttgccaccaaattggctgtatctgttctaagatggatcagaaaatcagttccaaagttggctacaaactttcaggtttgggttttgttttgtttttttgttttgttttgttttgtttttgcaaccagccaattcatcttagttcacatgacagagaagtgcataattacttgcaactttagttagagcagtggccttaagaaggtctagctaaataaaaagtgctcagactttctgagtgctgacagttgtcaaattcacctagttcacatggccccatttctatcgtttgttttgttttgtttttgttttttaacagcccatctgtgagcaataggatcagatgactaagagctacagggcagaaacactgttacttagagtcaaattttcccattacctagctgtaaagagtttgtttctctctgactcatataaagtttaccatttaggcccctgcatgattttaattccatcacttaacaccccagccatatgattctgaaggtaaacatgaaggcgtttgaattccagaccacctaaacattcttaaggaaatcatcatctccacgggcagagctatgccaaaatctgtaggttttaactcaaatttcatgataagcaaaaattgaattaatttgtcttccattttgttcacctttttgccaaaattatgcctggattagaataaataaattcaatcaatgaatgcaatcactaattcttacgccagataataacacattcagaattctcctttccctgggagattttatcaggttagtgttcttgtaaacaggagaaagagaaaaatataacttagtaaatagcagtattcactaattcattcatttattcaacaaatattaatttactacctactacattccagggagcttagagtctagtatcagaaataataaccacacacacacatacacacacactacattaaataaggatgtgataggctagatgaaataaataaataaataaaaggtccaggtgagaaaagaaggtgggggctagaaagaagtcattgaagaaaaaacatttaggttaaaacattatgaataacttagagtgagccaagtgcagagtgctgaaggagtgctccaggcaaaatcaacagcaaatggggagtccttgatgtagaaaagggtttgaggaattgtcctgggagaaatactcaagattccagtctgaattctagaggttagtgatttagagaggcaagtacgaaaatgacttcctctcttaccttaaaagtaagtgcaccatagaaggaaatcacccttccttggtaataattcctgagtgagcctgagaagccagaggccatctctattttataggcactgtccccttttcagttacccatggctagctcattgaccttgtcctggtcgtttcctcatttcacttactccatcctcaaaacgtagacgcttcataaatattgtataaatgaatgaactcacaaagtcacagtacagcaaggcaaaagtgcctgcaataaacaagcattctaggctagaaatatttctcaacttcaaattgtgtcttattacattgtattccgattttctagagtggtagttctcagtcaagggaaagtttttcttcccttccaggggatatttggcattgtctggagatagttttagttgtcacgatttgggggatgcttctggctcaacttgggtagagaagcggggatgcttataatcatcctacagtgcacaggacagtacccccacccacactccagtaatgaagaatcattagacctaaaatgttaatggtgtccaggtagaaaaaccctgttgtagaggttggggactgcgtcttgacagccacattatacagtgtatcaaacaattctgtataatgggctgtaattatccttgcctagattttgcaagaaccctagtgtgtatctttttcctcacttgccaagcaatgttcaaacctgcagagatttatttcattcattttctgtgtgtttagtaaacagactagaagcactggaggaaaaaatattccagcaatgaggtaagacgaaagctattagtaaccctagtttaacttagctgaatagtaggaaacaacctctaccgtgaggaagtgtattgtagaaactgaaaagacgctaatgatgtttaaaaagctgtagttcaaacaaatgtgcatgcagaccaatgggtagactgaaaatgatgaagacatttccgtttcttgtgtctttgatagaaaagaaagagcttttattttctttagtgtggcaatcattcagatttgtcccatgacatgcccagaaggttgaagaataacaaactcccaagtgtaaacacagaatttagcgaagaatccaggcctctggatgaatccctgtaattgcatgtttggataaaataagattttcatacattaaacaaggtaggatttttctatctgggacggaactttcaacacttggaggggttgtagttatttctcctcaaagatggcaaacatgagtgccccgagttatccctcctctctgttcaagttcgctaactaatcacccagtatccatgctatcgctggcccttctgtggcctatttttatactgttcactgttcagtgtcacttgtttggtaacactcaacatcaacatgtgctaccaaattgacaccagaggacaaaaaagaatcaagatatgtacagcctgctttgtactgagccagctgccactagatgttttttgtgataatgaacacgtgaggccatgtggacgcgagagatggctccgggttccctcagacggctcacagccagctggtctgcagtgcggttttagattccgatgtgggaaccccataaaaaagaatatgcaggccaggcgtggtggctcatgcctgtaatcccagcaatttgggagcctgaggcgggtggatcacctgaggtcaggagttcgagaccagcctcgccaacatggtgaaatcctgcctctactaaaaataaaaaaaaaaaaaattagtcaggtgtggtggcggatgcctgtaatcccagctacttgggaggctgaggcaggagaatcgcttgaacctgggaggcagaggttgcagtgagcaaagatcgcaccattgcacttcagactgggcaacaagaatgagactctgtcacaaaaaaaaaaaaaaaaagtctgcaggctgcataaagaggtatgaaaatgttccagaaatcccaaatcctatccctgaggttcattttggtgagggaatgtgtgtgcattttctagggcttccctaaaaaagtatcacaagctggatggcctaaagctacagaaatttcttggggacaaatttcatgattctggaagctagaggtccaaaatcaaggtgtcagcaaggctatgctttttctgaagcctatagggaaggccttccttgtctctcctagtttctggtggtttgctggcaatgtttggcattctgtggattgcagctacataactccactctgcctccatcattaatggccttctgcctgagtgttttcatatgaccatcttcatataaggacaccagtcatatttgatgagggttccaccctactccagtatgacctcatcttcactaactacatctgcaatgaccctatatccaaataaagtcacattctgagtgtctggggattagaacttcaacagagcttgttgaagggggcacaattcaatgcataacaggatggaaactagaaacgggtatgtttttatcagtgtagaaagatttagcttaatttttcaaagtgtaataaaaaccccaggaaaactcatactccctcctaagaagagcaaaagatggagaaacccgatggttaccttcaaacaaaaggaaaggaggaataagatgaaaaggaattaatccaaagcaaagagagtggcttatatggaatgttggtgcaactttctctgacacatctgtgcactcatcagctggggcatcatctccctggggtacatttggtcactgtgtgcctcatggtaataaactccagaagcctcattgacttgctagagatgagctcatccttcttgcttgcttaatggcaaaatacaaaataagcagtcactgacatggaacgatttcaggaatgccaaaaggttctccttttccaaaatatctcttccatcttcccaatactgttactgacatcactaacacctctccacttccggttgagacacctgggccagagctcctgatgtggcaggcagtgccctaaacgttttgcataaattaactgatgcccagagcaacaaccctaagatataggtactatcataccgcatcttacagataagaaacttaggcacaaagaggtttagtagtttagatgagataaccctgatgagcagagattcgaacccagcctccatgctattaaccaggacatcatattgcctttcatacatgctcttcaaaggcaacacagtaatcgattatcacactcactcacatctgattgtcacatttttcagatctgctctcctagcagagaatgaagcctaaggtatccttgtttctcaaagtgtcctccccagaccagctgcatcaaaatgaggggatgaggtgcaaatgcctggaccctgcccttggagcactgattcataatctcaagtcccaagaatctgcattttaacaagcatccccagaaatttcttaagtatactaatgtatgggaaccactgacactaaagaaatggaataaggggaacgtacaatgttacagtaaaccaggaaaagccagaaagacatgacaacacagtgaggactctggtagccaatggtcagtcaaatgcccaggggccctggccagaagagagttaggttgctgaggagtaagagtgatgctgaatgtggaggcttgagagcagaaggaagccagccagctatatcctcttgcttggatcacacaccctttccttggtggaaatggttatttgcagagttagagaaggcatgttttacagtttggatggcaggtatggatgtagacaataaagagcaaccagagtccatgggttcagaaatccccatgtgtttctgtttgaatgagacgcttgcataaacagcacaaggagtttggggtggggttaaagagaatggtgtggtatagggagagctgaatgaggaactgagagagcaaaatcctgtgtttggttcaatcactgattacaacctccctgaggctcggtctcctaatctgtaaaatggggggaaataatacctgccttgcaggtcctcacacacagggcatgatgtgaatccactgaggcatatagcactgtgtaacatgagttattgctattccaaggcccgtaaaaggctcttgccttggaatatatctgccacaccaatgcctgcagtccattaatgacacataaaggacactggagataacgatgtcccttgttctatgcatccctcccacccatgccagaaaagaaaacacagtcacctgaagtcattctaaagagtatgcctgcctcttttcctgcacagacacatatacacagacacgcacatacacagaccatgcacatacacacacatgggaaaacatgaggaaaagtggagacaagaggcaccaaaggacaaagtcacttttgtcgcctgtcccttccccagcagggctgggcctgggctgcttctcctgcctcctccctgaagccccctcctcatcatattccagtgcgtgtccaccactttggggccaggtctacacaactgcagtgattcaggtcacgggagaaaacccaaacaagcacaaaacatgcttcaacctatattttctaaattgtttttctttaaaggtgaagacttctgagcttgaattatccccttgtcagtgggctttccatgctgtccaagtgacctaagtgataatcaacctccatttcattttgagaatggttgtggtattttagagctatggtgaataagaaaatcatttaaaataaaatgatttttatttatttattgtttttatttattttatcttaaatgaattttaaatcatttaaaataaaataatgggataaaagaggatgctaaaaataataaatatatatgtatcaaagtgtgcttgtaataccaggcaaagaattaataagagataatattatggttggtgaaatgttatgtatggctacatcctttcaatgagcatttatagttcctttaaaatatgcctactgaagaaatatttacatgctaattaacatgtgcatagtaccactaggtattatagaggataccagatgtttgtagtagacacagaccttgccctaagtcctggtcttgatgtagtcactttttagtcactacaggtgactacatttagtcactacaagtgaccttccttcaatggggaaataaaggactttacaaaagacgtagaagacaattcttaatataaaagtgatttagatcttcacaagtttgtgaagagaagcagatgagtgaaatagaacactatcaatgtaaaatattattctgaggcctctgtaatgactgggaagcaacaagagggaggtcatttcagagagagaggctctaggttccaagctggatgctcaggtcagtgactgcaggtcccctccacacccatcaccccacaccctaaccctcttcagttgctcacaaaggtagataaatacccacatttttgccctcttccatcttgaaaccctggaaacccttgcttccgccaggggaggttacttagtatctgtcaccccaagggaaccaacgtcgaagcccaagaataagagtcaatactcctaccagaggtttacatttttcccaggggtctaggtggatattcctgggaacccccgtcaacacaggcatctacagtacaatccaggcctcctgttttcagcaggggctgcaagagcactgcagccttttccccagaggtgtcagtttggcccagtaaagattgcccctgagaaaacacatgggcaattagagcaaagttcctatgttctggtaacatttaattgtgctatttctcaacctcctctgcacccacacactcacacacaacatttattccactgacttcaaaggaagctcaacgtgttaaaaatatgtgtgggaacaaagaagggagtttgaaattggtctaaactctgtataactgggtttgacacgtacattaggattttacaagtatgtatttaatctttttttaaaaaaagcgtttacatagggttcagaataatgacaataaatcaacatttctattgtccatttgtgtgttttcatagtaaataatgctcatttatccttaaccagtaatacatacttatgggcttaaattagcaaaagcctctcaaaaagtagctccactcatttatccaccagtgtccagatgccatccagcacatgaggagctcccagaaaggagcagggaacaaactagggctgtcaggagtggaggagaaagaatggcatatgcaaaaaggagctgtaattaaatccaagggaacatggcacactctagtcttttgcacgagacaaagggcaatcctggtaaaaatacagatccccaggccccaccccaaagagtctgatctgattctgaaatggggccggagaatctgcattttaacaagcaccttcaccaggtgatccttttgctgagaacccctgagaaatgagaaccctgtgctagtgctgaatggagcattatattccagagttgaagtttggtgatcagttttccagatggagctggtccttggtgcatacctgggtataaatccaagccaattcaggtatatgagctgatatttcaaccgaaacactatctatagcctaaattttttctaatattctgtttggtatgaattctagaaagttgtaaatgctatatttccttctcatctatttctggactttgtcccaagaccaaatcccagggcatctgatagacattcattgcatacatttttctgtaaacatgaaaactgaattgtctaatagaaaagggcaaggaagtagaaaataagaaatcatcatcagaagtggtttgttttggaattatattgtccagctgcataacaaatcacccccaaaattgagtcgcttagaacaacaaacattgatcctccacagtttctgtgtgttaggaatcaaagtgatttaatttaatggttctgctcagggtctctcgggggctgcaatccaggtctcaggctgggatcctttcaaggctgagctggggaaagatccatgtctaagctcactcacatggccgatggcgggattcagttcctcttaggctgtcagactgagggcctccgtgtctcagtggttttagccagagccctctctcagttcctttccacatgggcctctccacagggcaactcacaacatggcagctggtttccagtagagcaagcgagtgagagaacaagaaaggcaagcaaggtgaatgtcccagtcttttgtaacctcatctcagaagtgttaacccatcacttttgccatgttttattatttagaagcaaatcactaagtccagcccacaattagagggatggcattacacaagggaatgaacaccagcagacagggtcattgaaagccatcttagatgctgtctatcgcatctaagtgtgatttttccagatgaaaagaatatattaatttgtttcagtcttagtcgatgtgccatcccatttgtgctttgctaaaacttgtatcaatgtaaagcaaacattttctgatacaatttaggtagtgtattgtggtaatagagaccagtagtgttgaaaagatatgttgaggtcagaaattaagctcatgtttctaaaagaggagatatgtacaactactatgcaagccaacaggaaagagtgttttaagaatgctttctgctacaggtaactaaaaacctaaacagctgtggctttaaaataaaggtatatctaagtcacataagcaaaagtctaggggtgggcagctgctggcattgcttcagtagcttgataatggcaaaagcagcatctcttctatttccttggccttctaatcatgcatgtcacctcacaatcacaacataggcaacacctcatattctaagcaagatgaaaagggcaaagagtcatgccatatgcctctgtctcttttcataaggaagacaaagcttccctggaagtcccctctagcagatttcacttagatctcattggccagaactgagtcacatgcctgccttaaaccaatcactcaccaagaagactaacattatcatggcaagtctaaaccaactgtgactcatctctgaaatcaaaggattattaccattacccgaatccatcaggatcctgttggcagagaagtgggactgtaaattttgagcaggcaacaaacaagtcttctgtaaacttcttatgtgttgttttttatgtgttctatatatccagtagaatcacaatttccaataacagtctaaaaagatattttccaatagaaacagaatgtgtaagatcattacttatgaaatcccaaatgtacttaaggtttccttcttgaaaattccttattcaaaataaaatgtccagattttgaaacccagaaaagattctatattttaaaaatcctgtgcacatgtaaactgtttttcaaatattgccttcagatacattgaacagaatgaaatcttctgagatttactacatcagccaagtattatcaaaacaaacaggacagattgcttttcttgacgtctgctgcttgatttgtgttaactcatgtttctgaaattgtagtatcataagccaatgctgcacaaaggtatttcatgtcatttataaaaatctagtaatgtaaactgttaactccttataaagcatctgttgacacacaaaaatatcactgaagtgcatttatgcctttcttctttaggtctgcataatacttccctccagaaggccaagttgttccataaattacagaacagaaagttggttgtgggaggaatagctcaacctcatctgaggcatcccactctaagaaactaatggcacctacacctcttgggcattgagtttttaagcccatttttaattcttgttctgctcatattctaagtgagcacataaagtgctgctccaagcaagaccagcccttgtagaagggcaagtgcagtcagtcccctaggaaacgggactggggagtgatcgtttcaatgagagataaatcaaactgatgctaaacatgaacaatgagcccattagagattgtgagaaagaggcatcatcatccactcaacaataggcctgtgggacctcttgatagcctgaggatgtttaatttcaggtgcaggtatccagaatgtagcagctagactgatcaaggatgtgtgatgacagcaagcagtagtggaagagcccaggagagttcctaagcctgaattgcaatcctgtgctgccataaaatgggaagatatacttggtccagtcatctgacagctttggtcatcaatttctctatctcatatgtgactctattgctttaagaatccctttagctttaaatatctatgaatctgctgaagcagctgtgctttgattgatgtggatctctgaactcccttaaatacaaagaccaattatttagccgagctttgttggattcagtgcattctgaatacatgtcaaaatatacttggatttgtaaaaaatattccttcctgtttttttcaccatagatagatgtacaaaaatgtccgtgttcacaccgtggaaaggacatttctcataaactcacacagagatacctttcaagtcaatgccttagaaagcaatgagagatttaaaggagacctagagatatgaatggagtaggcagagaaggtatgtgaggagaatgatgtaacttcctagggaaaaagtatgaagcacaaggctggacatagacctgggaatcaggaaattagagttctaattgcagcttttccattgattcacttgggatcttgagaatatctgtctcattttaatcattctgggccacagtttccatatctgtcaattagagtaagagtccctggctgggtgcccaggattgtgagaacataccattcagagccataaaaatgcaatcagtaccaataatgtactagtaccagtacctaggatgcaaaacatcctagatactaggtgtcctaacttaaagtggaaacattaacaagagtaattctttgaatcatcaaactgggaatattttaggaagcatatctatctgggtgaaaactaagcaaataagacaattgtaaaggcttgtgatctcaggaatacaaaggcaaaaatgcgcagacttgaaatatgacaagttctagttttgtcacttagcatctctgtgaccttggataatttcttaacccccggcagtattctcatctgtaaaatgggaataatgacatgcacttcagtggtttgtggtgaagattattacaaatagaaattagctcttttgagccactggtggggtttaaattcccagcccttatgtgctttgcagctgttagttcctcttattacaattgtctatttaaaaacctagtcacagcccggtgcagtagctcacgtctgtaatcccaacactttgggaggccaaggcaggagaactgcttgagctcaggcgttcaacatcagcctaggcaacatagtgagaccctctcatctctacaaaaagcaaaaaattagccagtgatgcatggctgtagtcccagctattctgagggctgaagttggaggattgcttgagcccaggaggtcaaggctgcagtgggcagtgatcatgccgctgcactctagcctggatgacagagcaagaacctgtctccaaaaaaagaaaggaaggaaggaaggaaggaaggaaggaggaaagaaaagaaagaaagaaagaaagaaagaaagaaagaaagagagagagagagagagagagagagaaagaaagaaagaaagaaagaaagaaagaaagaaagaaagaaagaaagaaagaaagaaagagaaagaaagaaagaagggagggagggagggagaggagagaaagaaaaaggaaggaaggaaggaagagagagagagagagagaaagacctagtcaccaaaagcaagagattttttaaatgctactattttttgggcatttactaatcatattgctatgctctgcacccaagctaagtaatttaaataaattatctcatgtactcctctaaaactaattactgctgtgtaaatggaggtagaaagaaactaagctttatttctgcctctattgtttctttaacctgccttgcttcctttttcagttgcacctaattggctgtacttttagttttctttaaaactgccttaaatttcaaagactaaagcagcaataactaactgaatatatttatataacatgttatttttgtcatgttgctttccacccctggagacctgctctaaattcacttggacgtttgaggataaatcatgctcactagcagtttctgaaaatgcagtttcactgaaaatgcaggcatccagaaatttagtaagcaacttaaaagaaagtgtaagaatctcctatgtattcattgaaaaataatttgaatttatgcttagaaaaatagaattattattaagaaatcttacacactcatgtttttaaatatcttcactaaggaccaattgtgtatatggtgtaacactgtcctcaaagaacatgccgggagaattgttgcagttaccagagggttaaatttggcaaactcttttttattaacgtgccttttaattatgaaatagcatactcaccttagataaaatttgaaaaccatttttgtaaagtggtacaatattgaagaaagttgataactttcagaccagatttaagcctcaaatctacctctcttttacctggacaactcattagcatttctgaacctcacattttttctataaagtgagaatactatattatagagttgttgtcagttaaatgagaacagtgtctgatcacaactagtcaacaaatgttcacaactcttcccctcctaggaaaagaatctcaaggcagacctgcttcgggtctgctctgtaaagaggtaggaatcctctgctcccggtaaattgcttcctaaccttctttggtaatagactattttttaataaaggtgatggatcatttcccattatacactcaaaatgtgtgtccatttcagggcagtcatggatgaccattgcccatcttttgaccccagattaagaacacctgctgtagtattttaattctgccttcaaatcctcttacaaaacaaagacatctttaaaaaataaaattctttaggtgtcttgcagttgaatgcaggaaaaccagagccccttatttttgatagttttgggaagaatgcagtgtcagaacacaaacccataatagacaaataatttgcacagaaacttcataaaagtattgacctgatttgccatgtatttgccaccttttaaaacacacaactaaatgtttaccctgtgtctagatccaaatgggtgaagaaaaatgagtgacaatacatctacttaagctcacttacataattgtggccatgccgtttttttcacattacattattagaacattggacaataagtcaagaaacagaatgttctacaaaataaactttaaaaattggtaagcatcatgtgctttttccagaagacattttattttgttgaatcaaaggtggctctttggcactgagtagctccgtggagtcatggcagtcctcattccctaatcctgagcctgcctgagtcgctgctgtcagtcatccacttgttgggatttcaaactgcattaaatcccctcctatagctgtcactgccaagcagttgcactggctctgtcctacctttctgttggtaattctgtttttaatcctgtgcttcagtgtagtttatataaatctttacagagggataaaacttcctgtaattaattgtttgggtgaacatgtacctgggagagctattgggaaaggggccaaatttgcattccagctcctttcatccccacccttgagctaaccaagtcctgtggattcttcccttagcatctctggaaccttcttttcttttcttttttttatgaccacctttccagtcctggcccttcaaacttgagtgacagcaacagtctccctgccttgagtctctttcctccttctcccagtgtgcatacggttgtcaaactcatcttgataaactactgcatcgattgtggctacactcccctgctcccacatcttccatagaccccactgtctgtaaaataatattcagtctggcctcaacctgtctttccagcctcggtgacacaggtctattctgcctgagacacttactatgacacccttgcttgttcctggggctttgacacatttccaacgtcccattgttcttcctctccaaatcagccaattgcccaagccctgctcaaatctcccacctcatgaagccttcttgatgcctcccagcacaccatgatctaatttcctgaagtaattatgctaattgggcatttgaagaattgttaaccgattatcaactaactgccccttaacattgcatgtgtagttgtcttcaaaggcagttaaattatgtcatgttccttacattgtactgagtgcctcgtatccttatccatgtttgggggttttactttaagtcaagaaatttaatcacatccatttggttttctctagagctgtagttctcaaccttttgtgtggtagagaaacacctagagaacatgtttaaaaatatcctgggttccacccttgagagataataaggtccaaggggaacccaaatatctgtgtttcaggtcagcttattggctcatcctattataccaactcctcagaaggccaaggtgggtggattccttgatctcaggcgttcaagaccagcctgggcaatatcgtgagactccatctcttaaaaaaaaaaaaaaaaaggattagccaagtgtggtggcatgaacctgtggtcccagctacttaagaggctgaggcagacagattgcttgagcctgggaagtcgaagctgcagtgagccatgatcatgccactgcactccagcctgggtgacagagcaagaccctgtctcaaaaaaataaaaatgaaaaaaatctgtgttcccaagttccaagtgatgctgatgctgctggttgcctttaagcatctcacaaagaacgaactcataaatgctaatacagtatatgtctatggatactgaatagtgggttttttttctcttttcttctattctgtgctcatgttgtgtcacttcttccttttagattgactttgaagatgtgattgcagaaccagaagggacacacagttttgacggcatttggaaggccagcttcaccaccttcactgtgacgaaatactggttttaccgcttgctgtctgccctctttggcatcccgatggcactcatctggggcatttacttcgccattctctctttcctgcacatctgggcagttgtaccatgcattaagagcttcctgattgagattcagtgcatcagccgtgtctattccatctacgtccacaccgtctgtgacccactctttgaagctgttgggaaaatattcagcaatgtccgcatcaacttgcagaaagaaatataaatgacatttcaaggatagaagtatacctgattttttttccttttaattttcctggtgccaatttcaagttccaagttgctaatacagcaacaatttatgaattgaattatcttggttgaaaataaaaagatcactttctcagttttcataagtattatgtctcttctgagctatttcatctatttttggcagtctgaatttttaaaacccatttaaatttttttccttacctttttatttgcatgtggatcaaccatcgctttattggctgagatatgaacatattgttgaaaggtaatttgagagaaatatgaagaactgaggaggaaaaaaaaaaaaaagaaaagaaccaacaacctcaactgcctactccaaaatgttggtcattttatgttaagggaagaattccagggtatggccatggagtgtacaagtatgtgggcagattttcagcaaactcttttcccactgtttaaggagttagtggattactgccattcacttcataatccagtaggatccagtgatccttacaagttagaaaacataatcttctgccttctcatgatccaactaatgccttactcttcttgaaattttaacctatgatattttctgtgcctgaatatttgttatgtagataacaagacctcagtgccttcctgtttttcacattttccttttcaaatagggtctaactcagcaactcgctttaggtcagcagcctccctgaagaccaaaattagaatatccatgacctagttttccatgcgtgtttctgactctgagctacagagtctggtgaagctcacttctgggcttcatctggcaacatctttatccgtagtgggtatggttgacactagcccaatgaaatgaattaaagtggaccaatagggctgagctctctgtgggctggcagtcctggaagccagctttccctgcctctcatcaactgaatgaggtcagcatgtctattcagcttcgtttattttcaagaataatcacgctttcctgaatccaaactaatccatcaccggggtggtttagtggctcaacattgtgttcccatttcagctgatcagtgggcctccaaggaggggctgtaaaatggaggccattgtgtgagcctatcagagttgctgcaaacctgacccctgctcagtaaagcacttgcaaccgtctgttatgctgtgacacatggcccctccccctgccaggagctttggacctaatccaagcatccctttgcccagaaagaagatgggggaggaggcagtaataaaaagattgaagtattttgctggaataagttcaaattcttctgaactcaaactgaggaatttcacctgtaaacctgagtcgtacagaaagctgcctggtatatccaaaagctttttattcctcctgctcatattgtgattctgcctttggggacttttcttaaaccttcagttatgatttttttttcatacacttattggaactctgcttgatttttgcctcttccagtcttcctgacactttaattaccaacctgttacctactttgactttttgcatttaaaacagacactggcatggatatagttttacttttaaactgtgtacataactgaaaatgtgctatactgcatactttttaaatgtaaagatatttttatctttatatgaagaaaatcacttaggaaatggctttgtgattcaatctgtaaactgtgtattccaagacatgtctgttctacatagatgcttagtccctcatgcaaatcaattactggtccaaaagattgctgaaattttatatgcttactgatatattttacaattttttatcatgcatgtcctgtaaaggttacaagcctgcacaataaaaatgtttaacggttaaacagtca

SEQ ID NO: 8 - DNA, 2723 bp, Homo sapiens
gggagaaacgttctcactcgctctctgctcgctgcgggcgctccccgccctctgctgccagaaccttggggatgtgcctagacccggcgcagcacacgtccgggccaaccgcgagcagaacaaacctttggcgggcggccaggaggctccctcccagccaccgcccccctccagcgcctttttttccccccatacaatacaagatcttccttcctcagttcccttaaagcacagcccagggaaacctcctcacagttttcatccagccacgggccagcatgtctgggggcaaatacgtagactcggagggacatctctacaccgttcccatccgggaacagggcaacatctacaagcccaacaacaaggccatggcagacgagctgagcgagaagcaagtgtacgacgcgcacaccaaggagatcgacctggtcaaccgcgaccctaaacacctcaacgatgacgtggtcaagattgactttgaagatgtgattgcagaaccagaagggacacacagttttgacggcatttggaaggccagcttcaccaccttcactgtgacgaaatactggttttaccgcttgctgtctgccctctttggcatcccgatggcactcatctggggcatttacttcgccattctctctttcctgcacatctgggcagttgtaccatgcattaagagcttcctgattgagattcagtgcatcagccgtgtctattccatctacgtccacaccgtctgtgacccactctttgaagctgttgggaaaatattcagcaatgtccgcatcaacttgcagaaagaaatataaatgacatttcaaggatagaagtatacctgattttttttccttttaattttcctggtgccaatttcaagttccaagttgctaatacagcaacaatttatgaattgaattatcttggttgaaaataaaaagatcactttctcagttttcataagtattatgtctcttctgagctatttcatctatttttggcagtctgaatttttaaaacccatttaaatttttttccttacctttttatttgcatgtggatcaaccatcgctttattggctgagatatgaacatattgttgaaaggtaatttgagagaaatatgaagaactgaggaggaaaaaaaaaaaaaagaaaagaaccaacaacctcaactgcctactccaaaatgttggtcattttatgttaagggaagaattccagggtatggccatggagtgtacaagtatgtgggcagattttcagcaaactcttttcccactgtttaaggagttagtggattactgccattcacttcataatccagtaggatccagtgatccttacaagttagaaaacataatcttctgccttctcatgatccaactaatgccttactcttcttgaaattttaacctatgatattttctgtgcctgaatatttgttatgtagataacaagacctcagtgccttcctgtttttcacattttccttttcaaatagggtctaactcagcaactcgctttaggtcagcagcctccctgaagaccaaaattagaatatccatgacctagttttccatgcgtgtttctgactctgagctacagagtctggtgaagctcacttctgggcttcatctggcaacatctttatccgtagtgggtatggttgacactagcccaatgaaatgaattaaagtggaccaatagggctgagctctctgtgggctggcagtcctggaagccagctttccctgcctctcatcaactgaatgaggtcagcatgtctattcagcttcgtttattttcaagaataatcacgctttcctgaatccaaactaatccatcaccggggtggtttagtggctcaacattgtgttcccatttcagctgatcagtgggcctccaaggaggggctgtaaaatggaggccattgtgtgagcctatcagagttgctgcaaacctgacccctgctcagtaaagcacttgcaaccgtctgttatgctgtgacacatggcccctccccctgccaggagctttggacctaatccaagcatccctttgcccagaaagaagatgggggaggaggcagtaataaaaagattgaagtattttgctggaataagttcaaattcttctgaactcaaactgaggaatttcacctgtaaacctgagtcgtacagaaagctgcctggtatatccaaaagctttttattcctcctgctcatattgtgattctgcctttggggacttttcttaaaccttcagttatgatttttttttcatacacttattggaactctgcttgatttttgcctcttccagtcttcctgacactttaattaccaacctgttacctactttgactttttgcatttaaaacagacactggcatggatatagttttacttttaaactgtgtacataactgaaaatgtgctatactgcatactttttaaatgtaaagatatttttatctttatatgaagaaaatcacttaggaaatggctttgtgattcaatctgtaaactgtgtattccaagacatgtctgttctacatagatgcttagtccctcatgcaaatcaattactggtccaaaagattgctgaaattttatatgcttactgatatattttacaattttttatcatgcatgtcctgtaaaggttacaagcctgcacaataaaaatgtttaacggttaaacagtcaaaaaaaaaaa

SEQ ID NO: 9 - protein, 178 aa, Homo sapiens
MSGGKYVDSEGHLYTVPIREQGNIYKPNNKAMADELSEKQVYDAHTKEIDLVNRDPKHLNDDVVKIDFEDVIAEPEGTHSFDGIWKASFTTFTVTKYWFYRLLSALFGIPMALIWGIYFAILSFLHIWAVVPCIKSFLIEIQCISRVYSIYVHTVCDPLFEAVGKIFSNVRINLQKEI

SEQ ID NO: 10 - DNA, 29546 bp, Homo sapiens
taccgggcggaggtgagcgcggcgccggctcctcctgcggcggactttgggtgcgacttgacgagcggtggttcgacaagtggccttgcgggccggatcgtcccaggtgagctgcggccgggactcctgggagctgtccggggtcgagggctgagccgcggggaccccccgagctctgcggggacgggcaggggacagacgcgcggcctgggcccgactcctcctgggctctggcgagggcgtctcggtggaagctcccaggaggcgcaggcgctggcgacaacaccccagctgccggactttggggcgccgggggctgcggtcggatcgtcctggggtccccgctagctagctgcggtgtgcgcttctgtggccggggataggtgaattgggcacggggcccttcttagctcctttgctatcagagtaactcgcacctcttttgcagtggaagagttgtaaatttgcttctggccttcccctacggattatacctggccttcccctacggattatactcaacttactgtttagaaaatgtggcccacgagacgcctggttactatcaaaaggagcggggtcgacggtccccactttcccctgagcctcagcacctgcttgtttggaaggtgagccccgcgggcgcgcgcggacgttttagctgagaaagaggaaaataccttagtcacagaataaagtccagaaacgcgctctaggattgggtcctgccgtcacttttccttggtgcttctcccattcgttactaagttgacatagttgtgtttttttgttttgtaagtataaatttgatgctagtttgtatgtttaagtggttttaaaaatcaagccaattaaaaaaatcgatttgctaatgttgcggtaaagaaagatgtagatgatcttcatatgtcactggctgcaggcaggcgtctgaagacactgtgcgcccccgggtgcctccacagtgggcatccctggccactggggacacagagaatgaaggaaggaagccatacacttgtctcttggctccttgtggcaataggaaaatgggacagaaagtcttcctgcctggaattcgagaacgtttcctcttatattgctgtcctgtttggtggtggtaataaccctgtccctgtataacaggtatagttgctgacagtgccccatcagcctctgacatgtgtcccactagaggcctgagaaggggggtcatttcccgtgtggtcatttcccagaccacacgggattcaggcctcaccctgtcctcccaaccccatcccggcctcaccctgtcctcccaaccccatcccctaccttgttcctcccgcctaaggcattccaagcctttgttcaccttggtacttcttacacacttggaagtttacaatgggaaagttctccccttcctcaagcttgtgtgatttccatcattcaggcatcaggtgaaatgtcacctctgaggaagccttgcatgaaaacttgtatttccaaccccacagcttcaggggtgagttgtgggtttgtctcccactaggctgagctccccaaaggcaaagactgtcttgttactaatcacgtgtgtagggccagggatggtgtctggcatagggggtgtgatcaatacctagcattctggcaagggtcagtagaaacatgggctggagccatggatttgcaatctcttgagaacagtggttctcacaggagggtcatctaaccttccaggattcatttgaatttccatgtcacaatataggacaggggcatgcgctgtcatcgcggaggagaggtcagggtgggcatcctaccaggctcagggcagcccccacaatacagaagcatctggcccaagatgtcaacagtgccaaagctcagaaacacccatctatagtcgtcctcctgccaaacaaattctcagttgtaagggatagtctttcctttgctctgatgtcctgttaccttaaaatcaaattattttattggggttaaggaagcttttcactttccatagatacctttctcttgaaaaggaaaaatataaacatttcatctccagtggcagtccttttgcttttttatacagtactccttgaatatatcttcatgcaagattttataatttagaaataattcctaagtgtttggtcacatgacctgggtaggaagagagattcttagactccaaaggttcagatggagaacagacaggccattgcatagtttattcaaatttttaagatcacaggctagaacaggaacactgaagactccaagaacctactaacaaactcttctgattattagtttgtataggtttaggaattgtctcttctgctactttgccatggtgttagctttctgtggaaaaacaagttgaatttagattttttttttgtgtcgcttgcaaattagtatttttattgtttagagacttgaatctgtcagtaaattcagtgtagttttaaaacaacaatgaaaagcttacccataatgagcatttactatgtgctaggcaaattagtgacatgtacaaccatttaattgaacatcacatgatgtaggttctaatgtaatttctttattgtatatgaagaaactgatgcacagaagtgctagcatcaaataagcacatttggggatgattaaacctctaaacatatttaacattacaaacaaacgtttttgaaaaagcacagagtattaaaaatagtcattatccaaaattgatgtaagtattcctttgtgtcttttaagtatagcttttcatataaaaggtaattcgatttgtgttctagtgttttcccttgtttttacatactctctgttttaaagatctaatagttacatgtttaatacttccaaaagttctttgcaaaatgcaagatcgtttacattgcccattttgtttgttactcctttatgtcatatttacagatcaaacattctagtcaaattgtgttttattttgggaaattgaggtaatcttttaaaaagttgcagcaaccaaaaaaaccttcttcttttgcatgaaggggtattgaatgtgacatccgtatccagcttcctgttgtgtcaaaacaacattgcaaaattgaaatccatgagcaggaggtgagtgggttttttgaagatctacttttaaagctttttagttcacttttaatctgttcattagttacatatttccccttcttttcacttgcaggcaatattacataatttcagttccacaaatccaacacaagtaaatgggtctgttattgatgagcctgtacggctaaaacatggagatgtaataactattattgatcgttccttcaggtaggtaaaagctgattggtctaacagtaaatgtcatcttctgattaaccataaactagtgtcaaagtcaactgtcattcttctttaatgttcatggatgtcaaagtatttcaggttttcattctcaacactaatcttaatttaaaaggatatggatctaggaagatgactaattgggacaaaatgtgatggaattcaaggatttgggtgcaaataattatgcagagggaggttgaccttgtcctaaaggagtttgttattttcttccaagtgttttgaaactgcatgagatttttctattcttttttattttaaagcaaaatatacacagagaaaaaggtacaaaacaagatggacaggtcaatttatcatgtgaacacccttttgatcaccacctattatggactgatttgtgctctcccgaaattcatattttgaattcctaacccctcgtaccttaggatgtaactgtatttggaaagagggcatttaaaaaggcaatgaaggctttttgagctattagtatgggccctaatctgatgattggtgtcattataagtggagattagggaacacgtgcacagaggaaagcccatgagaagaggcagcaagaaggcagccatcttcaagtcaaggagagaggcacagagaaatccaatccagctgacaccttgctcttggacttccagcctccagcactgagaagggcatgtctgtttttcaaggcattcagtctgtgggactttattatggtggccctagcagactaatacatcctcagctcatatagaacatttccagaagctttttaaaaatgctgttgtctaatcactacttcattccctcagcaagtaaaccaccgttttggcttttaggatatcataaacgtctcgagtttcactacctaagcatggatctctgaacacgatagttttgtctgttcttataaaagtccttaacagtgatacaagaatgtatacatttgtgcctggcttgtgcttttctgtgtgtgagattcatagcatgttggacatctctgtagtttgttccttctcatggcataagtatttcactgaaaaaaaaatgtttaatatatacaataatatacataactatacagaattcattcattcttgatggacatttgactaatgtctagtctttgaaatgtgtcaaggtttgctttatgagcagatatggctaattatcttactaatctgccttgtgtaattgaaaactgtattctacagtgtctcatatatatgtaatatataattattatatatatcacttatataatatataattataatatattatatataatcatatataattataatataatatataatattatatagtatatactatatgttatatagtaatatataatataataatatatattatatataccatatagtatatactgtatacagtatatactatatggtatatataatacatatacaatatatatacaatatatttatattatataatatataatatatacatatataatatgtaatatatatacacatatacagatacacacactcccgttagattcaaatctataatcttagtaaattttcccctactattattctaagagatatgttcaaatcacctactgaggattgattgattgatttgtttttatctctatcagtttttgctttgtatattttgaggctgtactatggtgtacaaacttagaatgatgtcttggtgcatggaactctgtcatttagagatgtcctctgtttttagtagtgctttcttttctgcttaatgtttcctttggatgatgagtatggttaaactaatttccttttgatgagtgcttgtgtagtataacttttccacccccttaattttaacttttcataattgttatggtttaagttgtctcttggaacctgttgtcaggtgacctcttgtgtttaactggaatatcttcttgctggattgatccttatataaaatgtcctttttctctatgaataacttttgtctgagatgattttgtctcatgttagtacagtactccagttcccctctgcttgctgtttgcatggtatatcttttcccatgcttttattttaaaagattgtgtctttgaatcaaaattgcgtctcttgtagacaacgtattagttgggtcatttaaaaatccattctgctaaatgaatctctgttattgtcttcatttgtattagctgttttttaatgcaccacttaaattccctagtcatttcttttactgtgcttctgggaagtttgccttcatttttaaagagtatttttattgaatacagacttctatgtaggaagtttttctttttttctttttctttttgtttgtttgtttgtttgtttttgagacagagtcttgctctgtcacccaggctggagtgcagtggtgtgatcttggctcactgcaacctccgcctcccgggttcaagtgattctcatgcctcagcctaccaagtagctgggattacaggcacgcgccacaacacccggctaatttttgcaatttttagtagagacggcgtttcaccatgatggccaggctggtcttgaactcttggtctcaagtgatccacctgcttcagcctcccaaaatgctgagattacaggcatgagccactgcgcccggcctataggtaggaagcttttgatgtttattggagggctgaactatttttcaaaacaagatattatccaaattgagataaattcagatgattaaagacatccaacaaagatttgaaagtgaatcttagaaagagagacttgattccaaactctctaccatgagaaataatatcagatgtgtgactgcttcagatcttacttaggttgcctataaattcaaatcaggtcatttcactgaggccaaaagttttcattatgcaaaataaaggattgcttctatgacttgatcactgaggtttgccaggacaattatgtatcttgctaaaggtgatcttcttaagatgtacctgtcttgttcagatacagactgaatgtgtgggaaactgaagtgatcatagtgttctaaaatataggtttgtagttaccctgtagtgtaagtgtagctttttatttaccctgaagtatgtaacttcacaacgtgagaaagattcagttacctacaggcttcctggctggccttccaagccatccgatgatttccatggacttaaactagaagaaacttgactttgctctcatgtgcattagcttatgtcatagaaggtgcttggtaatattgtcaaatcatacctgggctgttcgtcagtgatcagaagttaatctgggtccccagcctctgacagtggtatttcaaaacaaactcgatgcaactaatcctagtttttagtgtaaaagcacttttctaactctaaatttatttctttgtataaggtgatgtcttgaactaaacccaaagtttgttcgttgatccttggacattaagtggatatggagacttccgccccagggtataaatttttaatcaggatctttgtcgttctataaactgcaggtatgaaaatgaaagtcttcagaatggaaggaagtcaactgaatttccaagaaaaatacgtgaacaggtgatagaaaatatccaagtttgggctgattcgtttcgatgatatgaaatcatttatgtatgctttaatgatttcttacttgaaatcagttacaaagagggtggttactgttatagtaggaaaatgtaactaaagtataaaaacgagttgaacttagacgtattaaaataatctaaaatgatatttcctctttattgttcaaaatcagagcactaagttggtcttacacttttagccaaaaagtctattagagcatgttgcgagctttttgcaaacccatgattaggataaccccaacacgtagagctaacatttgggaattgcattatctggtggctcattttgtcgcccagtcattgtactcatccgtctttatgtgtctgaaataatcacattatgaaaatcatgagtgccaaagatgcttctctattttgatagatagacgacccatgaaacaggaaaagggatcctgtgctttgtcttgtgagagaatcctatagtttgagtgtatcagtgtggaacaggtattccaattgtacagcttatgaaaacataattgtcgacacgtgcttcgcaatttagatagtttgagtatatcagtgtggaagaggtattttagttgtacagcttattaaaacacagttgtcggcatgtgcttggaaatttagtgggatttaggcttaggatgagccctgagaattcatatttttcctacattttagagtgctcctaaagctagtttcagatgagctccttggaatatcttggcttaggagagttatcttaggtgcacctgaaattagcagtaggtactcttgaatttcaacatttctgcctttttttttttttttttttctgagacggagtctcactccgtcacccaggctggagtgcagtggtgcgatcttggctcactgcaacctccgcctcctgggttcaagcaattctcctgcctcagcctcccgagtagctgggactacaggtgcacaccaccatgcctggctaatttttgtatttttagtagagacagggtttcaccatattgaccaggctggtcttgaactcctaacctcgtgatccacccacctgggcctcccaaagtgctgggattacaggcttgagccaccatgcccagtccatttctgccatttaagtactgccttcttccctagaggagcagccttaatgttgaatctgatggtcatcagttcccgtgctggaagccagtttctgaggcatcccacactgtagccaccaggcaacttgctagtctatgggagtaaataaaccaaaaagccagttgaactgtgggttagatatgtgccactcagcagtctaggaactgtttgttctgttactggtctgtgatatagtgagtactgaaatggagtatgtagtaaatatgacagcaattcgacagcaattttgtggctattgaatgtaatcgtacaacaaaagtgagattatatactttacatattcataattttattagtggttcatttatattgtttagtcataagattcttgttccacttcagattggaaataaaaaaccatccctcacatagatagttttagaagcaaggtgttacgtatcaagaaacctaaaattgattaaggaagttagattctttgcctttgttaatattttttagatattttcttaataatccagatgataagcttttcttcctttggggcatcagacttttataaaagttatatatatatgttattctttagttcttccacccttccggaaataattccatatgaaggaaagttagggcgctgtctctggggagccaggtgggggcgctgcgcctgctcccaggctgtgaggttgcagaatctgtgaatataccatgtgtgcctgtaccatgaatttacctagggtgaatctaggctctcagatccaccctaagtatccaccagccaagagggcgcacatgccaagtggagcctcagttcatcgacaggtcctatggcccatttatgagaaaactgatgacgcagtcagtagttctgagctcgtggggggttacagatcttcagtataggtgatgtttaatattgactttggttcttccgcaataggatgttgggaaatatctttaaaggaagttataaaaacacaccgaggtttgattttagtaattttaagcctcgaatttgtttcctattaatcactcactattttttaatgagtcagctaataagtacttttatcctgaccactagctcccaactgacttgagctcatttctcaaacagtctttaaaagtcatatacgagtggtttggtaaagaaaacaattttttattgtttttatttttattttttgagacagagtctcgctttattgcccaggctagagtgcagtggtgcaatctctgctcactgcaacctctgcctcccgggttccagcaattctcctgcctcggccacctgagtagctgggactacaggcacctgccaccacatccagctaatgtttgtatttgcagtagagatgaggttacatcatgttggccaggctgttctcaaactcctgacctcaagtgatccgcccaccttggcctcccaaagtgctgggattacaggcatgagccatcatgcccagccagaaaacaataaataatattttcactgttgtgactgtcatcatttagaaagacctgcaaataactgttcttattttcccaacttacaggagccagcacgtcgtgtctcaagatctagcttctcttctgaccctggtgagtagtggttgggttttgttccagaatcctgaaagatcaggtctttgcgaagcatattggcaaagaaggggcaacaagaatgcaagtgctaaatgtctagcctgtcagaaatggaggaggtgtcatttaacatgtcaacgacagacagatgcctaactttcccttggtgactgatggtatgctatgcttaggagagactctagatatgagaaaattgcctgttcagctgtcaactcagttctcagtagttagatagcatgtgctatttcatactggttattggagctgatatgatttatatatttgtatcataatactagtatcataagacttgcaagttttaggcattaaataatttgtaggccaagtaaaagctagcttataagacaacctgaaacaattaaaatattgaattccacaaatgttaatcattgagaaaatgctgaacttctgtttcttatttcataattcattgccaagatgagaaagctcaagattccaaggcctattcaaaaatcactgaaggaaaagtttcaggaaatcctcaggtacatatcaagaatgtcaaagaagacagtaccgcagatgactcaaaagacagtgttgctcagggaacaactaatgttcattcctcagaacatgctggacgtaatggcagaaatgcagctgatcccatttctggggattttaaagaaatttccagcgttaaattagtgagccgttatggagaattgaagtctgttcccactacacaatgtcttgacaatagcaaaaaaaatgaatctcccttttggaagctttatgagtcagtgaagaaagagttggatgtaaaatcacaaaaagaaaatgtcctacagtattgtagaaaatctggattacaaactgattacgcaacagagaaagaaagtgctgatggtttacagggggagacccaactgttggtctcgcgtaagtcaagaccaaaatctggtgggagcggccacgctgtggcagagcctgcttcacctgaacaagagcttgaccagaacaaggggaagggaagagacgtggagtctgttcagactcccagcaaggctgtgggcgccagctttcctctctatgagccggctaaaatgaagacccctgtacaatattcacagcaacaaaattctccacaaaaacataagaacaaagacctgtatactactggtagaagagaatctgtgaatctgggtaaaagtgaaggcttcaaggctggtgataaaactcttactcccaggaagctttcaactagaaatcgaacaccagctaaagttgaagatgcagctgactctgccactaagccagaaaatctctcttccaaaaccagaggaagtattcctacagatgtggaagttctgcctacggaaactgaaattcacaatgagccatttttaactctgtggctcactcaagttgagaggaagatccaaaaggattccctcagcaagcctgagaaattgggcactacagctggacagatgtgctctgggttacctggtcttagttcagttgatatcaacaactttggtgattccattagtaagtttatttaattgtaaatttatttttctaaaagatgaacaccttctatgtagctgtgagaccttcattctttcagtagtgattagctttgtggaagagaatgtaagcacataaggcacagggataagacagacacgcacgctgttttcgctaatagatgaatactgaaggcctaggtaattgtaacaaggaatgacagaagagatggaagttgtcaggcaatcatgaagcaggggttattgaaggagatagggtggccccaaaaatatttgattctcctccatctatcacaaaatagaaaagagaataatcataattgcttttcacatggagtttaattctgaactacaatcaactgtatcatatgagtttggtcatgtggaataaatcactggtaatcattaaacatagcggtagttctcaggtgttgatactctgttttcagagtgtcctacaaacaatttgctgtaaacactggactgtacccatgagtccatatgccacatgaacacttgatccttggtgcaatagcagtgcccgtggctaggttgtctttttagagctatgtcacacatgaggaaacgcacaacccaaggccagtcaggaagtggcagaggtaggacctttatagcaggtgtggtagctcctcgggagcttcctacactcctagggactggctattagaagaaaagttgtctgggccacagacgcagtattcgtactccaggctgtgtttctgcaggtggcctatagcggtggggcatgaggctgtcgataaaataaggtttgtaacatcagtctcagtattccaccaagcttcctagtggggaagcatcacttaggcaagggatttacattaactggtggctcaggaaaatgtgtggccgggactgggctcctgtgaattgactcagaaccacctggaggagtttttacagctcatgcattctggtcactgggtggctcaggctgagacccaagtgcatgtctttagagagttctctggctgtcgtccccctactctaaggtgaaatctgctgctgcagactcctcctttctatgtccacacataatgtttcaatgttttaaagaaagcctctgtactgtccagtggcaacttgacgtcttagtggccactcccgcaatcctttaggaactcttagtagcttagcttcaccaggattgcaagaatagaggcgtgctgtaggtactgtgtttcgatgacttttcacttggtttgtctttaatgctaacgtgaaataaatcactagtcttggtatgttttttcattgctctttccacatttgtatccgttactttagatgagagtgagggaatacctttgaaaagaaggcgtgtgtcctttggtgggcacctaagacctgaactatttgatgaaaacttgcctcctaatacgcctctcaaaaggggagaagccccaaccaaaagaaagtctctggtaatgcacactccacctgtcctgaagaaaatcatcaaggtgagttgaacaagcagcatcccattcacgcccagtggctcacacaccgggtacctattcctttccaaatactgacacaagtagaattatgatgaagacctttatttatttattaaagaaacaagtttttaaaaagacgaatgaaagctcattgatgtgggcaccagcaaaggagcccctgggtccctgaccgtggtgctgatgcgaccgagtcgggggagtccttgatgttgtccttttcgtgcagaagttcccagacacccacgcttacagcactgcgtaatcacctgcaggcaccgtcagaatagccaattacaaagattctggatacattaccacgtcgacccataaggattgttttattttcttttagagttacaaggctatagttaaggcatttacctggtgtgaaggaaaatcaatggcacatcttccttttacaccttcagctctttagaatatttgcttgcctgttcctttgctgtgttgtccttcctgggtttcacacctctgggaagtcatggccctatagctcataacctttgtaatagggcctgggcggtctttctttccttctcttcccagcaggagtggaaagtgatgtctggctccctttccattttacactggctcccaggggcaagcagtcctccctccatgtggactgccgggggctgggtgctcacagctggtatttactgggactgagatcagaccctgcttgtctttcactgatcaggatgttccttccacctttttacctgaaatgacctcacttcagacttggcttgcttctctttccttttccttaaatgactgagacagtctcacctctaccatagtcttcaaagacagtggcttaagcatttgatgcttcaagatttaaacagctgaaattcttaactgaataggtttttatcagtcattccagagatgttagatccttgttaaggcttcttgtaaacaattttgtcttgtcaggaacagcctcaaccatcaggaaaacaagagtcaggttcagaaatccatgtggaagtgaaggcacaaagcttggttataagccctccagctcctagtcctaggaaaactccagttgccagtgatcaacgccgtaggtcctgcaaaacagcccctgcttccagcagcaaatctcagacagaggttcctaagagaggagggagaaagagtggcaacctgccttcaaagagagtgtctatcagccgaagtcaacatgatattttacagatgatatgttccaaaagaagaagtggtgcttcggaagcaaatctgattggttagtctgacattagaatacatatattaaggtgaattttttcatgaatgctaatttttgaaaagtttcgtcttttccctttgacagttgcaaaatcatgggcagatgtagtaaaacttggtgcaaaacaaacacaaactaaagtcataaaacatggtcctcaaaggtcaatgaacaaaaggcaaagaagacctgctactccaaaggtaggggcctcactgaatggctgctttacacataccggtgtcatcgaagcttgcattcagtgtggatttatgctttaatgtcctgtttacctccttcctgttttctacagaagcctgtgggcgaagttcacagtcaatttagtacaggccacgcaaactctccttgtaccataataatagggaaagctcatactgaaaaagtacatgtgcctgctcgaccctacagagtgctcaacaacttcatttccaaccaaaaaatggactttaaggaagatctttcaggtaaaaacgtagtcttagatcttataaatcttttgactctactgttttttactgtgttaatgtttgttttgctaactttgtttatctgctgtgaaagagattataaaataaacgactcaatcacccttctggggtcttagtgttcagggaagataaagctgttttggcttgcaaagacaagtctctgctaatttttgggatcacatgtcctgcatgaaataggtgtggaaatgtcaaatgcatgtctttaccctggagagcagcgtatacatagaaagctggaaggtgctttacagtgaagagagttaacttgttaatctgcaggtacaataatagtttatcccaggtgtgggccagttgttgttgagcaacattttgcttcttcaggattgactttctaggtagcttaactttcttgaaagaaagccctatgagaagattcccataaacgattgttgaagaaatatttccattcaattcccactctcagtaaattgtggcctgacttgtgtaatttaaagtggggatggccggcgcagtggctcacgcctgtaatcccaacactttgggaggctgaggcctcctatcattccaaacagaatgataatgatattagattttcaaaaatatatattattacttgacagtactaaaaatgggcagctctttgtcattttctaaaatgtgggtacgtgttatttgtacttaaaatctagaactgagcacccaagactttgagaagcatgaaggcatttggcctctagatcctagcagggaggcctatgggtctcaggagcccgtcaccagcctccgtgctccagagctcacgtgtggggtcttgttaggaggacccagaggtcagctaacttcatttgtttcctttccaagaaaagcaaagcgaagagcataacatatcttctctgtgctctcttttttacaaaatgagtacattattaatggaatcactctttttatgaggcctatgtaccaggatttcaagggaggaatctactgttatttcccaccatttgcacgttacatgtgagggctgggatggttgtctgcatgttagcaatatgtcaatcacttttcaaatattttcatttaacaggaatagctgaaatgttcaagaccccagtgaaggagcaaccgcagttgacaagcacatgtcacatcgctatttcaaattcagagaatttgcttggaaaacagtttcaaggaactgattcaggagaagaacctctgctccccacctcagagagttttggttggtttccttgtttcctaacactgtgatgttttgggataggaacatacagtattatttttttacaagcagtatgatattacttctctaaaacaaatgttcctggcacaaaataccatttccgtcttctctgttctaattgaaagggagatttcaatcagcagttgaactaacattggcattcattgacttatatagaaagaaagactccgttaatgtgtgtaaatgacctgtgtacacttttggcttctttattgtaagtaaccagggtgacatagcaaaaaaatacaatggtgtctcaccatatttacatataaattactcatattcaaaattactgccaattacaaaacagaaagataacagtttgtatactgtgcaattattcctgctaaccttttaggtgggagcaggcccggggtaagtgtgagatgggagatgagaatctgtctaatctctcatttggcactggtcactgtattcctgtcacagagggaacagcttgctaccctgaagatgtttgcggtaggtaaatatttcctgtgtaatatggaccctaaccacataaggataatccatgttattttattggcgatttaaggcaagaaacagctgtttgtttctaatgtttgaaggaaaaaatgcctatatttacgtcaatgacacagggcatggcaacctttggaggaaccttctgaagcagtatttttaccttctatgatcatttcagcatctaactttgcttaatgatgaccccactatagcaccaaataacattattcttaattgtgcatggctcttataagacagatttatagacacataagtagcctcatgtcgttcagtgaatacgaagctaaatatttagttttttaccatattttatgaggctgtaatactcgattgacatgagacactaatagaatgtttttattgtgtatgtttttatttcaggaggaaatgtgttcttcagtgcacagaatgcagcaaaacagccatctgataaatgctctgcaagccctcccttaagacggcagtgtattagagaaaatggaaacgtagcaaaaacgcccaggaacacctacaaaatgacttctctggagacaaaaacttcagatactgagacagagccttcaaaaacagtatccactgcaaacaggtcaggaaggtctacagagttcaggaatatacagaagctacctgtggaaagtaagagtgaagaaacaaatacagaaattgttgagtgcatcctaaaaagaggtcagaaggcaacactactacaacaaaggagagaaggagagatgaaggaaatagaaagaccttttgagacatataaggaaaatattgaattaaaagaaaacgatgaaaagatgaaagcaatgaagagatcaagaacttgggggcagaaatgtgcaccaatgtctgacctgacagacctcaagagcttgcctgatacagaactcatgaaagacacggcacgtggccagaatctcctccaaacccaagatcatgccaaggcaccaaagagtgagaaaggcaaaatcactaaaatgccctgccagtcattacaaccagaaccaataaacaccccaacacacacaaaacaacagttgaaggcatccctggggaaagtaggtgtgaaagaagagctcctagcagtcggcaagttcacacggacgtcaggggagaccacgcacacgcacagagagccagcaggagatggcaagagcatcagaacgtttaaggagtctccaaagcagatcctggacccagcagcccgtgtaactggaatgaagaagtggccaagaacgcctaaggaagaggcccagtcactagaagacctggctggcttcaaagagctcttccagacaccaggtccctctgaggaatcaatgactgatgagaaaactaccaaaatagcctgcaaatctccaccaccagaatcagtggacactccaacaagcacaaagcaatggcctaagagaagtctcaggaaagcagatgtagaggaagaattcttagcactcaggaaactaacaccatcagcagggaaagccatgcttacgcccaaaccagcaggaggtgatgagaaagacattaaagcatttatgggaactccagtgcagaaactggacctggcaggaactttacctggcagcaaaagacagctacagactcctaaggaaaaggcccaggctctagaagacctggctggctttaaagagctcttccagactcctggtcacaccgaggaattagtggctgctggtaaaaccactaaaataccctgcgactctccacagtcagacccagtggacaccccaacaagcacaaagcaacgacccaagagaagtatcaggaaagcagatgtagagggagaactcttagcgtgcaggaatctaatgccatcagcaggcaaagccatgcacacgcctaaaccatcagtaggtgaagagaaagacatcatcatatttgtgggaactccagtgcagaaactggacctgacagagaacttaaccggcagcaagagacggccacaaactcctaaggaagaggcccaggctctggaagacctgactggctttaaagagctcttccagacccctggtcatactgaagaagcagtggctgctggcaaaactactaaaatgccctgcgaatcttctccaccagaatcagcagacaccccaacaagcacaagaaggcagcccaagacacctttggagaaaagggacgtacagaaggagctctcagccctgaagaagctcacacagacatcaggggaaaccacacacacagataaagtaccaggaggtgaggataaaagcatcaacgcgtttagggaaactgcaaaacagaaactggacccagcagcaagtgtaactggtagcaagaggcacccaaaaactaaggaaaaggcccaacccctagaagacctggctggcttgaaagagctcttccagacaccagtatgcactgacaagcccacgactcacgagaaaactaccaaaatagcctgcagatcacaaccagacccagtggacacaccaacaagctccaagccacagtccaagagaagtctcaggaaagtggacgtagaagaagaattcttcgcactcaggaaacgaacaccatcagcaggcaaagccatgcacacacccaaaccagcagtaagtggtgagaaaaacatctacgcatttatgggaactccagtgcagaaactggacctgacagagaacttaactggcagcaagagacggctacaaactcctaaggaaaaggcccaggctctagaagacctggctggctttaaagagctcttccagacacgaggtcacactgaggaatcaatgactaacgataaaactgccaaagtagcctgcaaatcttcacaaccagacccagacaaaaacccagcaagctccaagcgacggctcaagacatccctggggaaagtgggcgtgaaagaagagctcctagcagttggcaagctcacacagacatcaggagagactacacacacacacacagagccaacaggagatggtaagagcatgaaagcatttatggagtctccaaagcagatcttagactcagcagcaagtctaactggcagcaagaggcagctgagaactcctaagggaaagtctgaagtccctgaagacctggccggcttcatcgagctcttccagacaccaagtcacactaaggaatcaatgactaacgaaaaaactaccaaagtatcctacagagcttcacagccagacctagtggacaccccaacaagctccaagccacagcccaagagaagtctcaggaaagcagacactgaagaagaatttttagcatttaggaaacaaacgccatcagcaggcaaagccatgcacacacccaaaccagcagtaggtgaagagaaagacatcaacacgtttttgggaactccagtgcagaaactggaccagccaggaaatttacctggcagcaatagacggctacaaactcgtaaggaaaaggcccaggctctagaagaactgactggcttcagagagcttttccagacaccatgcactgataaccccacgactgatgagaaaactaccaaaaaaatactctgcaaatctccgcaatcagacccagcggacaccccaacaaacacaaagcaacggcccaagagaagcctcaagaaagcagacgtagaggaagaatttttagcattcaggaaactaacaccatcagcaggcaaagccatgcacacgcctaaagcagcagtaggtgaagagaaagacatcaacacatttgtggggactccagtggagaaactggacctgctaggaaatttacctggcagcaagagacggccacaaactcctaaagaaaaggccaaggctctagaagatctggctggcttcaaagagctcttccagacaccaggtcacactgaggaatcaatgaccgatgacaaaatcacagaagtatcctgcaaatctccacaaccagacccagtcaaaaccccaacaagctccaagcaacgactcaagatatccttggggaaagtaggtgtgaaagaagaggtcctaccagtcggcaagctcacacagacgtcagggaagaccacacagacacacagagagacagcaggagatggaaagagcatcaaagcgtttaaggaatctgcaaagcagatgctggacccagcaaactatggaactgggatggagaggtggccaagaacacctaaggaagaggcccaatcactagaagacctggccggcttcaaagagctcttccagacaccagaccacactgaggaatcaacaactgatgacaaaactaccaaaatagcctgcaaatctccaccaccagaatcaatggacactccaacaagcacaaggaggcggcccaaaacacctttggggaaaagggatatagtggaagagctctcagccctgaagcagctcacacagaccacacacacagacaaagtaccaggagatgaggataaaggcatcaacgtgttcagggaaactgcaaaacagaaactggacccagcagcaagtgtaactggtagcaagaggcagccaagaactcctaagggaaaagcccaacccctagaagacttggctggcttgaaagagctcttccagacaccaatatgcactgacaagcccacgactcatgagaaaactaccaaaatagcctgcagatctccacaaccagacccagtgggtaccccaacaatcttcaagccacagtccaagagaagtctcaggaaagcagacgtagaggaagaatccttagcactcaggaaacgaacaccatcagtagggaaagctatggacacacccaaaccagcaggaggtgatgagaaagacatgaaagcatttatgggaactccagtgcagaaattggacctgccaggaaatttacctggcagcaaaagatggccacaaactcctaaggaaaaggcccaggctctagaagacctggctggcttcaaagagctcttccagacaccaggcactgacaagcccacgactgatgagaaaactaccaaaatagcctgcaaatctccacaaccagacccagtggacaccccagcaagcacaaagcaacggcccaagagaaacctcaggaaagcagacgtagaggaagaatttttagcactcaggaaacgaacaccatcagcaggcaaagccatggacacaccaaaaccagcagtaagtgatgagaaaaatatcaacacatttgtggaaactccagtgcagaaactggacctgctaggaaatttacctggcagcaagagacagccacagactcctaaggaaaaggctgaggctctagaggacctggttggcttcaaagaactcttccagacaccaggtcacactgaggaatcaatgactgatgacaaaatcacagaagtatcctgtaaatctccacagccagagtcattcaaaacctcaagaagctccaagcaaaggctcaagatacccctggtgaaagtggacatgaaagaagagcccctagcagtcagcaagctcacacggacatcaggggagactacgcaaacacacacagagccaacaggagatagtaagagcatcaaagcgtttaaggagtctccaaagcagatcctggacccagcagcaagtgtaactggtagcaggaggcagctgagaactcgtaaggaaaaggcccgtgctctagaagacctggttgacttcaaagagctcttctcagcaccaggtcacactgaagagtcaatgactattgacaaaaacacaaaaattccctgcaaatctcccccaccagaactaacagacactgccacgagcacaaagagatgccccaagacacgtcccaggaaagaagtaaaagaggagctctcagcagttgagaggctcacgcaaacatcagggcaaagcacacacacacacaaagaaccagcaagcggtgatgagggcatcaaagtattgaagcaacgtgcaaagaagaaaccaaacccagtagaagaggaacccagcaggagaaggccaagagcacctaaggaaaaggcccaacccctggaagacctggccggcttcacagagctctctgaaacatcaggtcacactcaggaatcactgactgctggcaaagccactaaaataccctgcgaatctcccccactagaagtggtagacaccacagcaagcacaaagaggcatctcaggacacgtgtgcagaaggtacaagtaaaagaagagccttcagcagtcaagttcacacaaacatcaggggaaaccacggatgcagacaaagaaccagcaggtgaagataaaggcatcaaagcattgaaggaatctgcaaaacagacaccggctccagcagcaagtgtaactggcagcaggagacggccaagagcacccagggaaagtgcccaagccatagaagacctagctggcttcaaagacccagcagcaggtcacactgaagaatcaatgactgatgacaaaaccactaaaataccctgcaaatcatcaccagaactagaagacaccgcaacaagctcaaagagacggcccaggacacgtgcccagaaagtagaagtgaaggaggagctgttagcagttggcaagctcacacaaacctcaggggagaccacgcacaccgacaaagagccggtaggtgagggcaaaggcacgaaagcatttaagcaacctgcaaagcggaagctggacgcagaagatgtaattggcagcaggagacagccaagagcacctaaggaaaaggcccaacccctggaagatctggccagcttccaagagctctctcaaacaccaggccacactgaggaactggcaaatggtgctgctgatagctttacaagcgctccaaagcaaacacctgacagtggaaaacctctaaaaatatccagaagagttcttcgggcccctaaagtagaacccgtgggagacgtggtaagcaccagagaccctgtaaaatcacaaagcaaaagcaacacttccctgcccccactgcccttcaagaggggaggtggcaaagatggaagcgtcacgggaaccaagaggctgcgctgcatgccagcaccagaggaaattgtggaggagctgccagccagcaagaagcagagggttgctcccagggcaagaggcaaatcatccgaacccgtggtcatcatgaagagaagtttgaggacttctgcaaaaagaattgaacctgcggaagagctgaacagcaacgacatgaaaaccaacaaagaggaacacaaattacaagactcggtccctgaaaataaggtgagaggaagtattacagcatcactcaatatcgtcttggatagttgtgaatttctgtgttatactttgcatgtaacctgacgttataagtggttatcctttccccagagggtttcctttaacactgaataactgcaggccatggtaaagaatcttcttgggacctatagagggaatagactaatatgggaaaagaactagtcaaacatgattaatgcaaacttagaagagtttctcaaaagccaaatgagtaatgttcatcttcaaatgtttaaaaaattgatcagaggataggtgggtggttcagttctatgttctggatactgtcaccaagaccaggactgggttaaaaatcctatctatgtcactattttaactatcagaacaatatcaataaggaatgtgtattcttgagaaggggaaactgtctggactgttagatagagaggctcccactgcatgcagagaagtttggggaaactccagaaatggcaatttcctgttctgccttagcagtgatggttaaaggaataggaattcatttactccctgtaaatgaatgaataacggaccttctttctataatgtaaactgtaaaaattagatttgatatatatcttattaggcattgttattgaaatttatcctgaattccttatttataatttgtcagcagagacagtcatttctccataggagcaaagtaccaaggcattaccttgactatcctagatgacaagtctcttagacaattcatcagtgtgtgattttcaaatgatttctccttttaggttactgttgattgtgctccctgttgaattttgaagtttgttttactaattattggaattttgtggatgtcttctcatttctttagggaatatccctgcgctccagacgccaaaataagactgaggcagaacagcaaataactgaggtctttgtattagcagaaagaatagaaataaacagaaatgaaaagaagcccatgaagacctccccagagatggacattcagaatccagatgatggagcccggaaacccatacctagagacaaagtcactgagaacaaaaggtgcttgaggtctgctagacagaatgagagctcccagcctaaggtggcagaggagagcggagggcagaagagtgcgaaggttctcatgcagaatcagaaagggaaaggagaagcaggaaattcagactccatgtgcctgagatcaagaaagacaaaaagccagcctgcagcaagcactttggagagcaaatctgtgcagagagtaacgcggagtgtcaagaggtgtgcagaaaatccaaagaaggtaagccatttactgtttccctgttttaaaagacacagaatgttttgatgtattttttgcccaaggttgcaaaggcaaacaaaaagcattatcttcaacagcactctgccagctcccagttacattaatggaaggagcattatgctaagacataaatttggtcattttagttatattttgttgtgtctactttgggaatttcacttcaaatagcaaattcacgttatgaagactttcttcagaaagaaaaggaatttgtctagtggctaatttctgactgcttatgatgtacctcatgatacaattgtaggtagatgtcttcagcctttctgtagttcaaaatcacctcttgaaacaggaaaattctttttccaaaaaaaactattgaaacagaaaaatttccaaggcattctttggaaaggcagaaggaacagaagttgggatgtccctgagacacatgctcactatgtgagagttcctggtcagtgggggatgcgtctgccctccaaccctcctccagccagagactatctaagctaatgaactttaagtgctactgaaatcacatcaagttgagccactcataattacaagcatgtggacaaatccataagaatgattagattccattagtgttcttctggggaatacttggtttagctaaaataaaaataggttgaattgctccctttttgtgagcaattcacaaaaatataatataaatttaattcttagcacacacaaaatttggacctaaaactaccatattctgagttcagcaaacgaaactcatgtagcaataacctcttcagatacagaattctggcaagtacagctgtccagacagaccctcttcggcacaaactaggaagcttcaactgccaagaaaaggaaagattcccaggtgtccccagtacctgctgcatagccaggcagccagtggagggggagggcttgtgtgtctggatggtgggcaggaagtcctagtggggagatgcccatagactgttccagagagggaagaggagaggctcaagaagggcactgatgcccacacaggtgtcctaagtcagagactgtacttctggggcaggctggaagcagaagacacccgacagtgccagacaaggtcctagaaaccacaggcacaccagggacgcaacagctgacgtgggtcagcacgttcacccgtcgagtcttacaataaaacgcaggctccaggcgcttaccgaattcacagtctggaaggtgctgcttcctcagaatgaaaccaaagaagggtgaaagcgaatcttttaactaggatgattcctaaacacagagcaaccaggcccaaggctgagccaggcctggtgtgttctgccgggggcgttccagcctcttctcacacacaagcacccaggagacgcccaacacacacatgctcctctttgccgagctcttcggtgggtgtcggtagtcctagaaaatgcatatataagatagattttttccagtaagaagcttgtagccatgcttttcaccatggttcctctcccttagccaaggggtgagaactactgcaagagtaaaggccaaggcaggtctcctgcatgcaagttgggcatgcttctgtctacaggggttccttggtttaggagacccaaaagacttaatcctggttggattcactttttctgagtgacattttttagtttgtgaaaatgtgtgcatcgatgaagaaattttattatgaattagcttaaaaatgcattaggaacttctgtatgaaaagatcacattatttaagtgtaaaaaaactgcataataaaagcagttcaagtcaagaaaaacaatgttaatggaatatattttaaaacttatttccaacctcaaaattaattttctgcaactaaggacctgcataatacctagtaagcctttggggttttgcagaggaggtcgattctaaaaatgggtgtttaaattacttaagagttctattttttttcttcccacacaggctgaggacaatgtgtgtgtcaagaaaataagaaccagaagtcatagggacagtgaagatatttgacagaaaaatcgaactgggaaaaatataataaagttagttttgtgataagttctagtgcagtttttgtcataaattacaagtgaattctgtaagtaaggctgtcagtctgcttaagggaagaaaactttggatttgctgggtctgaatcggcttcataaactccactgggagcactgctgggctcctggactgagaatagttgaacaccgggggctttgtgaaggagtctgggccaaggtttgccctcagctttgcagaatgaagccttgaggtctgtcaccacccacagccaccctacagcagccttaactgtgacacttgccacactgtgtcgtcgtttgtttgcctatgtcctccagggcacggtggcaggaacaactatcctcgtctgtcccaacactgagcaggcactcggtaaacacgaatgaatggatgagcgcacggatgaatggagcttacaagatctgtctttccaatggccgggggcatttggtccccaaattaaggctattggacatctgcacaggacagtcctatttttgatgtcctttcctttctgaaaataaagttttgtgctttggagaatgactcgtgagcacatctttagggaccaagagtgactttctgtaaggagtgactcgtggcttgccttggtctcttgggaatacttttctaactagggttgctctcacctgagacattctccacccgcggaatctcagggtcccaggctgtgggccatcacgacctcaaactggctcctaatctccagctttcctgtcattgaaagcttcggaagtttactggctctgctcccgcctgttttctttctgactctatctggcagcccgatgccacccagtacaggaagtgacaccagtactctgtaaagcatcatcatccttggagagactgagcactcagcaccttcagccacgatttcaggatcgcttccttgtgagccgctgcctccgaaatctcctttgaagcccagacatctttctccagcttcagacttgtagatataactcgttcatcttcatttactttccactttgccccctgtcctctctgtgttccccaaatcagagaatagcccgccatcccccaggtcacctgtctggattcctccccattcacccaccttgccaggtgcaggtgaggatggtgcaccagacagggtagctgtcccccaaaatgtgccctgtgcgggcagtgccctgtctccacgtttgtttccccagtgtctggcggggagccaggtgacatcataaatacttgctgaatgaatgcagaaatcagcggtactgacttgtactatattggctgccatgatagggttctcacagcgtcatccatgatcgtaagggagaatgacattctgcttgagggagggaatagaaaggggcagggaggggacatctgagggcttcacagggctgcaaagggtacagggattgcaccagggcagaacaggggagggtgttcaaggaagagtggctcttagcagaggcactttggaaggtgtgaggcataaatgcttccttctacgtaggccaacctcaaaactttcagtaggaatgttgctatgatcaagttgttctaacactttagacttagtagtaattatgaacctcacatagaaaaatttcatccagccatatgcctgtggagtggaatattctgtttagtagaaaaatcctttagagttcagctctaaccagaaatcttgctgaagtatgtcagcaccttttctcaccctggtaagtacagtatttcaagagcacgctaagggtggttttcattttacagggctgttgatgatgggttaaaaatgttcatttaagggctacccccgtgtttaatagatgaacaccacttctacacaaccctccttggtactgggggagggagagatctgacaaatactgcccattcccctaggctgactggatttgagaacaaatacccacccatttccaccatggtatggtaacttctctgagcttcagtttccaagtgaatttccatgtaataggacattcccattaaatacaagctgtttttactttttcgcctcccagggcctgtgggatctggtcccccagcctctcttgggctttcttacactaactctgtacctaccatctcctgcctcccttaggcaggcacctccaaccaccacacactccctgctgttttccctgcctggaactttccctcctgccccaccaagatcatttcatccagtcctgagctcagcttaagggaggcttcttgcctgtgggttccctcacccccatgcctgtcctccaggctggggcaggttcttagtttgcctggaattgttctgtacctctttgtagcacgtagtgttgtggaaactaagccactaattgagtttctggctcccctcctggggttgtaagttttgttcattcatgagggccgactgcatttcctggttactctatcccagtgaccagccacaggagatgtccaataaagtatgtgatgaaatggtcttaaaa

SEQ ID NO: 11 - DNA, 12507 bp, Homo sapiens
taccgggcggaggtgagcgcggcgccggctcctcctgcggcggactttgggtgcgacttgacgagcggtggttcgacaagtggccttgcgggccggatcgtcccagtggaagagttgtaaatttgcttctggccttcccctacggattatacctggccttcccctacggattatactcaacttactgtttagaaaatgtggcccacgagacgcctggttactatcaaaaggagcggggtcgacggtccccactttcccctgagcctcagcacctgcttgtttggaaggggtattgaatgtgacatccgtatccagcttcctgttgtgtcaaaacaacattgcaaaattgaaatccatgagcaggaggcaatattacataatttcagttccacaaatccaacacaagtaaatgggtctgttattgatgagcctgtacggctaaaacatggagatgtaataactattattgatcgttccttcaggtatgaaaatgaaagtcttcagaatggaaggaagtcaactgaatttccaagaaaaatacgtgaacaggagccagcacgtcgtgtctcaagatctagcttctcttctgaccctgatgagaaagctcaagattccaaggcctattcaaaaatcactgaaggaaaagtttcaggaaatcctcaggtacatatcaagaatgtcaaagaagacagtaccgcagatgactcaaaagacagtgttgctcagggaacaactaatgttcattcctcagaacatgctggacgtaatggcagaaatgcagctgatcccatttctggggattttaaagaaatttccagcgttaaattagtgagccgttatggagaattgaagtctgttcccactacacaatgtcttgacaatagcaaaaaaaatgaatctcccttttggaagctttatgagtcagtgaagaaagagttggatgtaaaatcacaaaaagaaaatgtcctacagtattgtagaaaatctggattacaaactgattacgcaacagagaaagaaagtgctgatggtttacagggggagacccaactgttggtctcgcgtaagtcaagaccaaaatctggtgggagcggccacgctgtggcagagcctgcttcacctgaacaagagcttgaccagaacaaggggaagggaagagacgtggagtctgttcagactcccagcaaggctgtgggcgccagctttcctctctatgagccggctaaaatgaagacccctgtacaatattcacagcaacaaaattctccacaaaaacataagaacaaagacctgtatactactggtagaagagaatctgtgaatctgggtaaaagtgaaggcttcaaggctggtgataaaactcttactcccaggaagctttcaactagaaatcgaacaccagctaaagttgaagatgcagctgactctgccactaagccagaaaatctctcttccaaaaccagaggaagtattcctacagatgtggaagttctgcctacggaaactgaaattcacaatgagccatttttaactctgtggctcactcaagttgagaggaagatccaaaaggattccctcagcaagcctgagaaattgggcactacagctggacagatgtgctctgggttacctggtcttagttcagttgatatcaacaactttggtgattccattaatgagagtgagggaatacctttgaaaagaaggcgtgtgtcctttggtgggcacctaagacctgaactatttgatgaaaacttgcctcctaatacgcctctcaaaaggggagaagccccaaccaaaagaaagtctctggtaatgcacactccacctgtcctgaagaaaatcatcaaggaacagcctcaaccatcaggaaaacaagagtcaggttcagaaatccatgtggaagtgaaggcacaaagcttggttataagccctccagctcctagtcctaggaaaactccagttgccagtgatcaacgccgtaggtcctgcaaaacagcccctgcttccagcagcaaatctcagacagaggttcctaagagaggagggagaaagagtggcaacctgccttcaaagagagtgtctatcagccgaagtcaacatgatattttacagatgatatgttccaaaagaagaagtggtgcttcggaagcaaatctgattgttgcaaaatcatgggcagatgtagtaaaacttggtgcaaaacaaacacaaactaaagtcataaaacatggtcctcaaaggtcaatgaacaaaaggcaaagaagacctgctactccaaagaagcctgtgggcgaagttcacagtcaatttagtacaggccacgcaaactctccttgtaccataataatagggaaagctcatactgaaaaagtacatgtgcctgctcgaccctacagagtgctcaacaacttcatttccaaccaaaaaatggactttaaggaagatctttcaggaatagctgaaatgttcaagaccccagtgaaggagcaaccgcagttgacaagcacatgtcacatcgctatttcaaattcagagaatttgcttggaaaacagtttcaaggaactgattcaggagaagaacctctgctccccacctcagagagttttggaggaaatgtgttcttcagtgcacagaatgcagcaaaacagccatctgataaatgctctgcaagccctcccttaagacggcagtgtattagagaaaatggaaacgtagcaaaaacgcccaggaacacctacaaaatgacttctctggagacaaaaacttcagatactgagacagagccttcaaaaacagtatccactgcaaacaggtcaggaaggtctacagagttcaggaatatacagaagctacctgtggaaagtaagagtgaagaaacaaatacagaaattgttgagtgcatcctaaaaagaggtcagaaggcaacactactacaacaaaggagagaaggagagatgaaggaaatagaaagaccttttgagacatataaggaaaatattgaattaaaagaaaacgatgaaaagatgaaagcaatgaagagatcaagaacttgggggcagaaatgtgcaccaatgtctgacctgacagacctcaagagcttgcctgatacagaactcatgaaagacacggcacgtggccagaatctcctccaaacccaagatcatgccaaggcaccaaagagtgagaaaggcaaaatcactaaaatgccctgccagtcattacaaccagaaccaataaacaccccaacacacacaaaacaacagttgaaggcatccctggggaaagtaggtgtgaaagaagagctcctagcagtcggcaagttcacacggacgtcaggggagaccacgcacacgcacagagagccagcaggagatggcaagagcatcagaacgtttaaggagtctccaaagcagatcctggacccagcagcccgtgtaactggaatgaagaagtggccaagaacgcctaaggaagaggcccagtcactagaagacctggctggcttcaaagagctcttccagacaccaggtccctctgaggaatcaatgactgatgagaaaactaccaaaatagcctgcaaatctccaccaccagaatcagtggacactccaacaagcacaaagcaatggcctaagagaagtctcaggaaagcagatgtagaggaagaattcttagcactcaggaaactaacaccatcagcagggaaagccatgcttacgcccaaaccagcaggaggtgatgagaaagacattaaagcatttatgggaactccagtgcagaaactggacctggcaggaactttacctggcagcaaaagacagctacagactcctaaggaaaaggcccaggctctagaagacctggctggctttaaagagctcttccagactcctggtcacaccgaggaattagtggctgctggtaaaaccactaaaataccctgcgactctccacagtcagacccagtggacaccccaacaagcacaaagcaacgacccaagagaagtatcaggaaagcagatgtagagggagaactcttagcgtgcaggaatctaatgccatcagcaggcaaagccatgcacacgcctaaaccatcagtaggtgaagagaaagacatcatcatatttgtgggaactccagtgcagaaactggacctgacagagaacttaaccggcagcaagagacggccacaaactcctaaggaagaggcccaggctctggaagacctgactggctttaaagagctcttccagacccctggtcatactgaagaagcagtggctgctggcaaaactactaaaatgccctgcgaatcttctccaccagaatcagcagacaccccaacaagcacaagaaggcagcccaagacacctttggagaaaagggacgtacagaaggagctctcagccctgaagaagctcacacagacatcaggggaaaccacacacacagataaagtaccaggaggtgaggataaaagcatcaacgcgtttagggaaactgcaaaacagaaactggacccagcagcaagtgtaactggtagcaagaggcacccaaaaactaaggaaaaggcccaacccctagaagacctggctggcttgaaagagctcttccagacaccagtatgcactgacaagcccacgactcacgagaaaactaccaaaatagcctgcagatcacaaccagacccagtggacacaccaacaagctccaagccacagtccaagagaagtctcaggaaagtggacgtagaagaagaattcttcgcactcaggaaacgaacaccatcagcaggcaaagccatgcacacacccaaaccagcagtaagtggtgagaaaaacatctacgcatttatgggaactccagtgcagaaactggacctgacagagaacttaactggcagcaagagacggctacaaactcctaaggaaaaggcccaggctctagaagacctggctggctttaaagagctcttccagacacgaggtcacactgaggaatcaatgactaacgataaaactgccaaagtagcctgcaaatcttcacaaccagacccagacaaaaacccagcaagctccaagcgacggctcaagacatccctggggaaagtgggcgtgaaagaagagctcctagcagttggcaagctcacacagacatcaggagagactacacacacacacacagagccaacaggagatggtaagagcatgaaagcatttatggagtctccaaagcagatcttagactcagcagcaagtctaactggcagcaagaggcagctgagaactcctaagggaaagtctgaagtccctgaagacctggccggcttcatcgagctcttccagacaccaagtcacactaaggaatcaatgactaacgaaaaaactaccaaagtatcctacagagcttcacagccagacctagtggacaccccaacaagctccaagccacagcccaagagaagtctcaggaaagcagacactgaagaagaatttttagcatttaggaaacaaacgccatcagcaggcaaagccatgcacacacccaaaccagcagtaggtgaagagaaagacatcaacacgtttttgggaactccagtgcagaaactggaccagccaggaaatttacctggcagcaatagacggctacaaactcgtaaggaaaaggcccaggctctagaagaactgactggcttcagagagcttttccagacaccatgcactgataaccccacgactgatgagaaaactaccaaaaaaatactctgcaaatctccgcaatcagacccagcggacaccccaacaaacacaaagcaacggcccaagagaagcctcaagaaagcagacgtagaggaagaatttttagcattcaggaaactaacaccatcagcaggcaaagccatgcacacgcctaaagcagcagtaggtgaagagaaagacatcaacacatttgtggggactccagtggagaaactggacctgctaggaaatttacctggcagcaagagacggccacaaactcctaaagaaaaggccaaggctctagaagatctggctggcttcaaagagctcttccagacaccaggtcacactgaggaatcaatgaccgatgacaaaatcacagaagtatcctgcaaatctccacaaccagacccagtcaaaaccccaacaagctccaagcaacgactcaagatatccttggggaaagtaggtgtgaaagaagaggtcctaccagtcggcaagctcacacagacgtcagggaagaccacacagacacacagagagacagcaggagatggaaagagcatcaaagcgtttaaggaatctgcaaagcagatgctggacccagcaaactatggaactgggatggagaggtggccaagaacacctaaggaagaggcccaatcactagaagacctggccggcttcaaagagctcttccagacaccagaccacactgaggaatcaacaactgatgacaaaactaccaaaatagcctgcaaatctccaccaccagaatcaatggacactccaacaagcacaaggaggcggcccaaaacacctttggggaaaagggatatagtggaagagctctcagccctgaagcagctcacacagaccacacacacagacaaagtaccaggagatgaggataaaggcatcaacgtgttcagggaaactgcaaaacagaaactggacccagcagcaagtgtaactggtagcaagaggcagccaagaactcctaagggaaaagcccaacccctagaagacttggctggcttgaaagagctcttccagacaccaatatgcactgacaagcccacgactcatgagaaaactaccaaaatagcctgcagatctccacaaccagacccagtgggtaccccaacaatcttcaagccacagtccaagagaagtctcaggaaagcagacgtagaggaagaatccttagcactcaggaaacgaacaccatcagtagggaaagctatggacacacccaaaccagcaggaggtgatgagaaagacatgaaagcatttatgggaactccagtgcagaaattggacctgccaggaaatttacctggcagcaaaagatggccacaaactcctaaggaaaaggcccaggctctagaagacctggctggcttcaaagagctcttccagacaccaggcactgacaagcccacgactgatgagaaaactaccaaaatagcctgcaaatctccacaaccagacccagtggacaccccagcaagcacaaagcaacggcccaagagaaacctcaggaaagcagacgtagaggaagaatttttagcactcaggaaacgaacaccatcagcaggcaaagccatggacacaccaaaaccagcagtaagtgatgagaaaaatatcaacacatttgtggaaactccagtgcagaaactggacctgctaggaaatttacctggcagcaagagacagccacagactcctaaggaaaaggctgaggctctagaggacctggttggcttcaaagaactcttccagacaccaggtcacactgaggaatcaatgactgatgacaaaatcacagaagtatcctgtaaatctccacagccagagtcattcaaaacctcaagaagctccaagcaaaggctcaagatacccctggtgaaagtggacatgaaagaagagcccctagcagtcagcaagctcacacggacatcaggggagactacgcaaacacacacagagccaacaggagatagtaagagcatcaaagcgtttaaggagtctccaaagcagatcctggacccagcagcaagtgtaactggtagcaggaggcagctgagaactcgtaaggaaaaggcccgtgctctagaagacctggttgacttcaaagagctcttctcagcaccaggtcacactgaagagtcaatgactattgacaaaaacacaaaaattccctgcaaatctcccccaccagaactaacagacactgccacgagcacaaagagatgccccaagacacgtcccaggaaagaagtaaaagaggagctctcagcagttgagaggctcacgcaaacatcagggcaaagcacacacacacacaaagaaccagcaagcggtgatgagggcatcaaagtattgaagcaacgtgcaaagaagaaaccaaacccagtagaagaggaacccagcaggagaaggccaagagcacctaaggaaaaggcccaacccctggaagacctggccggcttcacagagctctctgaaacatcaggtcacactcaggaatcactgactgctggcaaagccactaaaataccctgcgaatctcccccactagaagtggtagacaccacagcaagcacaaagaggcatctcaggacacgtgtgcagaaggtacaagtaaaagaagagccttcagcagtcaagttcacacaaacatcaggggaaaccacggatgcagacaaagaaccagcaggtgaagataaaggcatcaaagcattgaaggaatctgcaaaacagacaccggctccagcagcaagtgtaactggcagcaggagacggccaagagcacccagggaaagtgcccaagccatagaagacctagctggcttcaaagacccagcagcaggtcacactgaagaatcaatgactgatgacaaaaccactaaaataccctgcaaatcatcaccagaactagaagacaccgcaacaagctcaaagagacggcccaggacacgtgcccagaaagtagaagtgaaggaggagctgttagcagttggcaagctcacacaaacctcaggggagaccacgcacaccgacaaagagccggtaggtgagggcaaaggcacgaaagcatttaagcaacctgcaaagcggaagctggacgcagaagatgtaattggcagcaggagacagccaagagcacctaaggaaaaggcccaacccctggaagatctggccagcttccaagagctctctcaaacaccaggccacactgaggaactggcaaatggtgctgctgatagctttacaagcgctccaaagcaaacacctgacagtggaaaacctctaaaaatatccagaagagttcttcgggcccctaaagtagaacccgtgggagacgtggtaagcaccagagaccctgtaaaatcacaaagcaaaagcaacacttccctgcccccactgcccttcaagaggggaggtggcaaagatggaagcgtcacgggaaccaagaggctgcgctgcatgccagcaccagaggaaattgtggaggagctgccagccagcaagaagcagagggttgctcccagggcaagaggcaaatcatccgaacccgtggtcatcatgaagagaagtttgaggacttctgcaaaaagaattgaacctgcggaagagctgaacagcaacgacatgaaaaccaacaaagaggaacacaaattacaagactcggtccctgaaaataagggaatatccctgcgctccagacgccaaaataagactgaggcagaacagcaaataactgaggtctttgtattagcagaaagaatagaaataaacagaaatgaaaagaagcccatgaagacctccccagagatggacattcagaatccagatgatggagcccggaaacccatacctagagacaaagtcactgagaacaaaaggtgcttgaggtctgctagacagaatgagagctcccagcctaaggtggcagaggagagcggagggcagaagagtgcgaaggttctcatgcagaatcagaaagggaaaggagaagcaggaaattcagactccatgtgcctgagatcaagaaagacaaaaagccagcctgcagcaagcactttggagagcaaatctgtgcagagagtaacgcggagtgtcaagaggtgtgcagaaaatccaaagaaggctgaggacaatgtgtgtgtcaagaaaataagaaccagaagtcatagggacagtgaagatatttgacagaaaaatcgaactgggaaaaatataataaagttagttttgtgataagttctagtgcagtttttgtcataaattacaagtgaattctgtaagtaaggctgtcagtctgcttaagggaagaaaactttggatttgctgggtctgaatcggcttcataaactccactgggagcactgctgggctcctggactgagaatagttgaacaccgggggctttgtgaaggagtctgggccaaggtttgccctcagctttgcagaatgaagccttgaggtctgtcaccacccacagccaccctacagcagccttaactgtgacacttgccacactgtgtcgtcgtttgtttgcctatgtcctccagggcacggtggcaggaacaactatcctcgtctgtcccaacactgagcaggcactcggtaaacacgaatgaatggatgagcgcacggatgaatggagcttacaagatctgtctttccaatggccgggggcatttggtccccaaattaaggctattggacatctgcacaggacagtcctatttttgatgtcctttcctttctgaaaataaagttttgtgctttggagaatgactcgtgagcacatctttagggaccaagagtgactttctgtaaggagtgactcgtggcttgccttggtctcttgggaatacttttctaactagggttgctctcacctgagacattctccacccgcggaatctcagggtcccaggctgtgggccatcacgacctcaaactggctcctaatctccagctttcctgtcattgaaagcttcggaagtttactggctctgctcccgcctgttttctttctgactctatctggcagcccgatgccacccagtacaggaagtgacaccagtactctgtaaagcatcatcatccttggagagactgagcactcagcaccttcagccacgatttcaggatcgcttccttgtgagccgctgcctccgaaatctcctttgaagcccagacatctttctccagcttcagacttgtagatataactcgttcatcttcatttactttccactttgccccctgtcctctctgtgttccccaaatcagagaatagcccgccatcccccaggtcacctgtctggattcctccccattcacccaccttgccaggtgcaggtgaggatggtgcaccagacagggtagctgtcccccaaaatgtgccctgtgcgggcagtgccctgtctccacgtttgtttccccagtgtctggcggggagccaggtgacatcataaatacttgctgaatgaatgcagaaatcagcggtactgacttgtactatattggctgccatgatagggttctcacagcgtcatccatgatcgtaagggagaatgacattctgcttgagggagggaatagaaaggggcagggaggggacatctgagggcttcacagggctgcaaagggtacagggattgcaccagggcagaacaggggagggtgttcaaggaagagtggctcttagcagaggcactttggaaggtgtgaggcataaatgcttccttctacgtaggccaacctcaaaactttcagtaggaatgttgctatgatcaagttgttctaacactttagacttagtagtaattatgaacctcacatagaaaaatttcatccagccatatgcctgtggagtggaatattctgtttagtagaaaaatcctttagagttcagctctaaccagaaatcttgctgaagtatgtcagcaccttttctcaccctggtaagtacagtatttcaagagcacgctaagggtggttttcattttacagggctgttgatgatgggttaaaaatgttcatttaagggctacccccgtgtttaatagatgaacaccacttctacacaaccctccttggtactgggggagggagagatctgacaaatactgcccattcccctaggctgactggatttgagaacaaatacccacccatttccaccatggtatggtaacttctctgagcttcagtttccaagtgaatttccatgtaataggacattcccattaaatacaagctgtttttactttttcgcctcccagggcctgtgggatctggtcccccagcctctcttgggctttcttacactaactctgtacctaccatctcctgcctcccttaggcaggcacctccaaccaccacacactccctgctgttttccctgcctggaactttccctcctgccccaccaagatcatttcatccagtcctgagctcagcttaagggaggcttcttgcctgtgggttccctcacccccatgcctgtcctccaggctggggcaggttcttagtttgcctggaattgttctgtacctctttgtagcacgtagtgttgtggaaactaagccactaattgagtttctggctcccctcctggggttgtaagttttgttcattcatgagggccgactgcatttcctggttactctatcccagtgaccagccacaggagatgtccaataaagtatgtgatgaaatggtcttaaaaaaaaaaaaaa

SEQ ID NO: 12 - protein, 3256 aa, Homo sapiens
MWPTRRLVTIKRSGVDGPHFPLSLSTCLFGRGIECDIRIQLPVVSKQHCKIEIHEQEAILHNFSSTNPTQVNGSVIDEPVRLKHGDVITIIDRSFRYENESLQNGRKSTEFPRKIREQEPARRVSRSSFSSDPDEKAQDSKAYSKITEGKVSGNPQVHIKNVKEDSTADDSKDSVAQGTTNVHSSEHAGRNGRNAADPISGDFKEISSVKLVSRYGELKSVPTTQCLDNSKKNESPFWKLYESVKKELDVKSQKENVLQYCRKSGLQTDYATEKESADGLQGETQLLVSRKSRPKSGGSGHAVAEPASPEQELDQNKGKGRDVESVQTPSKAVGASFPLYEPAKMKTPVQYSQQQNSPQKHKNKDLYTTGRRESVNLGKSEGFKAGDKTLTPRKLSTRNRTPAKVEDAADSATKPENLSSKTRGSIPTDVEVLPTETEIHNEPFLTLWLTQVERKIQKDSLSKPEKLGTTAGQMCSGLPGLSSVDINNFGDSINESEGIPLKRRRVSFGGHLRPELFDENLPPNTPLKRGEAPTKRKSLVMHTPPVLKKIIKEQPQPSGKQESGSEIHVEVKAQSLVISPPAPSPRKTPVASDQRRRSCKTAPASSSKSQTEVPKRGGRKSGNLPSKRVSISRSQHDILQMICSKRRSGASEANLIVAKSWADVVKLGAKQTQTKVIKHGPQRSMNKRQRRPATPKKPVGEVHSQFSTGHANSPCTIIIGKAHTEKVHVPARPYRVLNNFISNQKMDFKEDLSGIAEMFKTPVKEQPQLTSTCHIAISNSENLLGKQFQGTDSGEEPLLPTSESFGGNVFFSAQNAAKQPSDKCSASPPLRRQCIRENGNVAKTPRNTYKMTSLETKTSDTETEPSKTVSTANRSGRSTEFRNIQKLPVESKSEETNTEIVECILKRGQKATLLQQRREGEMKEIERPFETYKENIELKENDEKMKAMKRSRTWGQKCAPMSDLTDLKSLPDTELMKDTARGQNLLQTQDHAKAPKSEKGKITKMPCQSLQPEPINTPTHTKQQLKASLGKVGVKEELLAVGKFTRTSGETTHTHREPAGDGKSIRTFKESPKQILDPAARVTGMKKWPRTPKEEAQSLEDLAGFKELFQTPGPSEESMTDEKTTKIACKSPPPESVDTPTSTKQWPKRSLRKADVEEEFLALRKLTPSAGKAMLTPKPAGGDEKDIKAFMGTPVQKLDLAGTLPGSKRQLQTPKEKAQALEDLAGFKELFQTPGHTEELVAAGKTTKIPCDSPQSDPVDTPTSTKQRPKRSIRKADVEGELLACRNLMPSAGKAMHTPKPSVGEEKDIIIFVGTPVQKLDLTENLTGSKRRPQTPKEEAQALEDLTGFKELFQTPGHTEEAVAAGKTTKMPCESSPPESADTPTSTRRQPKTPLEKRDVQKELSALKKLTQTSGETTHTDKVPGGEDKSINAFRETAKQKLDPAASVTGSKRHPKTKEKAQPLEDLAGLKELFQTPVCTDKPTTHEKTTKIACRSQPDPVDTPTSSKPQSKRSLRKVDVEEEFFALRKRTPSAGKAMHTPKPAVSGEKNIYAFMGTPVQKLDLTENLTGSKRRLQTPKEKAQALEDLAGFKELFQTRGHTEESMTNDKTAKVACKSSQPDPDKNPASSKRRLKTSLGKVGVKEELLAVGKLTQTSGETTHTHTEPTGDGKSMKAFMESPKQILDSAASLTGSKRQLRTPKGKSEVPEDLAGFIELFQTPSHTKESMTNEKTTKVSYRASQPDLVDTPTSSKPQPKRSLRKADTEEEFLAFRKQTPSAGKAMHTPKPAVGEEKDINTFLGTPVQKLDQPGNLPGSNRRLQTRKEKAQALEELTGFRELFQTPCTDNPTTDEKTTKKILCKSPQSDPADTPTNTKQRPKRSLKKADVEEEFLAFRKLTPSAGKAMHTPKAAVGEEKDINTFVGTPVEKLDLLGNLPGSKRRPQTPKEKAKALEDLAGFKELFQTPGHTEESMTDDKITEVSCKSPQPDPVKTPTSSKQRLKISLGKVGVKEEVLPVGKLTQTSGKTTQTHRETAGDGKSIKAFKESAKQMLDPANYGTGMERWPRTPKEEAQSLEDLAGFKELFQTPDHTEESTTDDKTTKIACKSPPPESMDTPTSTRRRPKTPLGKRDIVEELSALKQLTQTTHTDKVPGDEDKGINVFRETAKQKLDPAASVTGSKRQPRTPKGKAQPLEDLAGLKELFQTPICTDKPTTHEKTTKIACRSPQPDPVGTPTIFKPQSKRSLRKADVEEESLALRKRTPSVGKAMDTPKPAGGDEKDMKAFMGTPVQKLDLPGNLPGSKRWPQTPKEKAQALEDLAGFKELFQTPGTDKPTTDEKTTKIACKSPQPDPVDTPASTKQRPKRNLRKADVEEEFLALRKRTPSAGKAMDTPKPAVSDEKNINTFVETPVQKLDLLGNLPGSKRQPQTPKEKAEALEDLVGFKELFQTPGHTEESMTDDKITEVSCKSPQPESFKTSRSSKQRLKIPLVKVDMKEEPLAVSKLTRTSGETTQTHTEPTGDSKSIKAFKESPKQILDPAASVTGSRRQLRTRKEKARALEDLVDFKELFSAPGHTEESMTIDKNTKIPCKSPPPELTDTATSTKRCPKTRPRKEVKEELSAVERLTQTSGQSTHTHKEPASGDEGIKVLKQRAKKKPNPVEEEPSRRRPRAPKEKAQPLEDLAGFTELSETSGHTQESLTAGKATKIPCESPPLEVVDTTASTKRHLRTRVQKVQVKEEPSAVKFTQTSGETTDADKEPAGEDKGIKALKESAKQTPAPAASVTGSRRRPRAPRESAQAIEDLAGFKDPAAGHTEESMTDDKTTKIPCKSSPELEDTATSSKRRPRTRAQKVEVKEELLAVGKLTQTSGETTHTDKEPVGEGKGTKAFKQPAKRKLDAEDVIGSRRQPRAPKEKAQPLEDLASFQELSQTPGHTEELANGAADSFTSAPKQTPDSGKPLKISRRVLRAPKVEPVGDVVSTRDPVKSQSKSNTSLPPLPFKRGGGKDGSVTGTKRLRCMPAPEEIVEELPASKKQRVAPRARGKSSEPVVIMKRSLRTSAKRIEPAEELNSNDMKTNKEEHKLQDSVPENKGISLRSRRQNKTEAEQQITEVFVLAERIEINRNEKKPMKTSPEMDIQNPDDGARKPIPRDKVTENKRCLRSARQNESSQPKVAEESGGQKSAKVLMQNQKGKGEAGNSDSMCLRSRKTKSQPAASTLESKSVQRVTRSVKRCAENPKKAEDNVCVKKIRTRSHRDSEDI

SEQ ID NO: 13 - DNA, 29430 bp, Homo sapiens
gattggctggtctgcttcgggcgggctaaaggaaggttcaagtggagctctcctaaccgacgcgcgtctgtggagaagcggcttggtcgggggtggtctcgtggggtcctgcctgtttagtcgctttcagggttcttgagccccttcacgaccgtcaccatggaagtgtcaccattgcaggtacggctcgcgggggggactggcggtggagcctccgcgcggcccgggcatctctctggccgcccgtgacgggtgaagctctggggctgcggtcaggccggcgaccggcttgggagcccatattctccattcccggttccggggtgatcgtggagaggcggaagccccttctggtgctagtagtgaagtatgacccggcttccagggtgtcgtgcgttgttgccctgtttctaggggcaggagtccgttggtcccgtagtggatccatgttacagcggcccgggtgcgacgttattgagtcgcgcgtacagatgctttcccctcctgcccgctgcttgaaaacggtcttgaatgtccccgatcttggaaagggcagagcctagcacagtgtttctctggagcagctggccagcttacaaaagcgcccccttcctgagttcacaacgctcaggtggccaagggattgaacggatagccgcccaaactgctgctgccgaactagaattatttacggtgtcgtgtgctcgggacaccgtaaataatttataataataggcactcggtaaataatgtatgcaggaactgaaagaagtgggtgtattttattgatgacatctaccatgttgctcaagaaaagaggtttccagacagacccacttggtgagataatatgtgtctgtttaacctgacacctggggacctggccgcagcgaatttaaattgtcctatttgtacaaaactggcaactccaccactgcctactaacagattgcaatgcatgttgacatctcactatggtaggcattttgttcctagtttagggtaaacttatgattaaaaattcccttctttctctctttttttttaattgctagcctgtaaatgaaaatatgcaagtcaacaaaataaagaaaaatgaagatgctaagaaaagactgtctgttgaaagaatctatcaaaagaaaacacaattggaacatattttgctccgcccagacacctacattggttctgtggaattagtgacccaggtaaaatcaagctcatcaaaggcagttaacctagtagttgaaatggatactcttctttctgtacctttcatgtactgagtgtgacctttctgtatctctcatgtactgagtgtcactggaactggcctaaatatggtaatacttaatatgaacactttatggtaatcatgataacagaacaatgaatatagaataaaaataacagtatttctttgccttatttctagcaaatgtgggtttacgatgaagatgttggcattaactatagggaagtcacttttgttcctggtttgtacaaaatctttgatgagattctaggtgagtaaatcctttaatataaagatctggtcaaatctttcataagaagtatcattccaaataaaaatttaaaaaagataaaagtaaaaacaaaaagtatcatttctaccaaacctgctgtagccagggctcctgtcatttcaatcagttgataaaggtgagctggtttcctggagcccaggaggttgagcttctcgtagaaacagtactatggacaaaaattgctaaattaacaccttgttaatgccactcctgtgctgtttgggaacttttttttctatatcttacttagagttacagaatgtttcttaaaacactaatttcttacaaatgtatattttattttctttcagttaatgctgcggacaacaaacaaagggacccaaaaatgtcttgtattagagtcacaattgatccgtacgtcttttgcttagtttttgttatgtagctcgttgctattgttagctgcatgtattttgattactggtgaagttggcttttcctaaacagaagttgagaaaagtacacaaaagcagaatgctaaaatgaaaaacctatggatacccaccacctagatccaacagttgccaacgtttttccatattttctccatcttttttttaattagttaatttttttagaggaaggggtcttgctattttgcctgggctggtcttagaactcctggcctgaagcaatcctccagtgttggcattatgggtgtgtgccatggcacgcggcctatttgctctggatcatttcaaaataaattacaggacttatgacacctaatccctaaatatttgagcaagcaccttcagtaattgctttaagtattctggcatctggagttttaactttttctcatgatgctattcaaacagtaaaccagtaggtggtggtagagaataatttgatctgacatttctgcttataaatgcggggtgtcccttagtgggtgatcagggtgcttatttcacttttttgttagtctgattgattatgacaaagtatacctggattttcctaaggactcaatatcatagtctttaaaaaatgttgagctagggccaggcacggtggctcatgcctgtgatcccagcactttgggaggccgatgcgggcggatcacaaggtcaggagatcaagaccatcctggctaacacggtgaaaccctgtctccactaaaaatacaaaaaattagccgggtgtagtggtgggcgcctgtagtcccagctactcgagaggctgaggcaggagaatggcatgaacccgggaggtggagcttgcagtgagccgagattgcaccactgcactccagcctgggtgacacagcaagactctgtctcaaaaaaaaaaaaaaagttgagctaattttaattagttttttttatagataataagtactatgtagaacaagaaccataatgtatatcgagttattaactaaaatatagtaccatccaagaattaacttaaaataactgaaataagttttcagcattttatttgagagcaaggcagtttatgattgatattggttttccttttttttttttttttttttgatggagttttgctcttattgcccagtctggagtgcagtggcatgatctcggctcaccgcaacatccacctcctgggttcaagcaattctcctgcctcagcctccagagtagctgggattacaggcgcacaccaccacacctggctaattttgtattttttttagtagagatggggtttctgcatgttggtcaggctggtctcgaactcctgacctcaagtgatctgcctgccttagcctcccaaagtgctgggattgctgggattacaggcgtgagccactgcgcccagcctgtttttatgtactttgttgagtgggaacgagatgtatagatgatcctcttgaaaggggtataataggcatgatgactcactcctgtaatcccagcactttgggaggccaaggtgggaggatcagttgagcccaggagtagtttgagaccagcctgggccatatagtgagaccctcatctctacaaaaaatttaaaaattagccaggtgtggtgatggacacctgtagtcccagctattcaggaggctgaagtgggaggattgcttgagcccaggaggtcaagactgcagtaagctgtgatcctgccattacactgcagcctgggtgacagagcgagatgctgtctcaaaaaaggagtgggagtggtgtataataatgtttccagattgtccttttctttccattgggcataaatcatttccctctgtttatcctaacattttaaaattttgtattttttcaacattatcagtgtctattcaggtgttggaaaaggtttttttttcctcttgactcctatttcaggaatgtcaggaactaagcatggtgggaaatgttcctgctgaatttcagaaggtatatatgaaaagtgccaaaaaaattgagggctgggtatggtggctcacatctataaccccaacactttgagcagttctcctgcttcagcctcccaagtagctggaatcacaggcatgcgctaccatgcctggctaattttttttatttttagtagagacggtttcaacatgttggccagaactcctggcctcaagtgatctgccctcctcagcctcccaaagtgctgggattacaggcacaggccaccgcgcccagcctgaatatacatattttagaaataatagtatttaaatatttttaaatgttaagcattcgtttaatgtgttgtaaaatacaattttcaatctttttttcttagggaaaacaatttaattagtatatggaataatggaaaaggtattcctgttgttgaacacaaagttgaaaagatgtatgtcccagctctcatatttggacagctcctaacttctagtaactatgatgatgatgaaaagaaagtgacaggtagagtattgaggggaaataacatatttgttgctaaaaatatatatatttaaatgactgtctgtggcatgagggttaaagatatggaaataaatctctataattgaatagctctgccagtgattaagaaataaagctgtcaatgagatagtaacaataaaatagtgtttcatatttatttgcccaggtggtcgaaatggctatggagccaaattgtgtaacatattcagtaccaaatttactgtggaaacagccagtagagaatacaagaaaatgttcaaacaggcaagtaaataagtgtcttgtaccttaatgataaatggtagtagtatagccatttataatggcattaatgattggtttaatttaacataatttataagctattgaagtatggaaaattataagcatatatattaggttattaggactcataaatttatgttatttacttccagtttgtgagatgacttgaatttttcatgtttcctattctttacttccatagacatggatggataatatgggaagagctggtgagatggaactcaagcccttcaatggagaagattatacatgtatcacctttcagcctgatttgtctaagtttaaaatgcaaagcctggacaaagatattgttgcactaatggtcagaagagcatatgatattgctggatccaccaaagatgtcaaagtctttcttaatggaaataaactgccagtgagtattttcctggatgttaaggataataagggattttgtaatcattgtcaagtgcaaaattgaattttttcccctcccatatgtttttgtttgtttgtttgtttgtttgtttgagacagagtctcacactgttgcccgggctggagtgcagtggcacgatctcggctcaccgcaacctccacctcccaggttcacgcaattctcctgcctcagcctcccaagtagctgggattacaggtgcctgccaccacacctggctaattttttgtatttttagtagagacaggtttcactatgttggccaggctggtctcgaacaccagacctcatgatccacccgtcttggcctcccaaagtgctgggattacaggcatgagccactgcacctggcccaaccatatgtattttcttaccacttctcacatatgttcttgaaaagagaatggtatgccacattttttaatcagctcattttaaacttaccgaaggaatttctttctcaaagaaacacctaaaataaatatttcatgtcctttttttattttcctttttctttcttttcttgataacctcgctgtgtcacccaggctggagtacagtgatgcaatcacggctcactacagcctggacctcccaggctcaagcgatcatcccacctcagcttctggagtagctggaaatgcaggcagcaccaccatgcccagctaatttttttttttctttttaatagaggtggggatctcactatgttgcccagggtggtcttgaactcctgggctcaagtgatccacccacctcggcctgtgtcctttaatgaccattcccttatgcctatcagtgaacatcattgcattggttttggaaagtcctcatagtctatcattgaacctattttttaataactttcttaatactgttacctttaattcctgtacaggtaaaaggatttcgtagttatgtggacatgtatttgaaggacaagttggatgaaactggtaactccttgaaagtaatacatgaacaagtaaaccacaggtgggaagtgtgtttaactatgagtgaaaaaggctttcagcaaattagctttgtcaacagcattgctacatccaaggtaattttattcttaaattattaatcatgatttatctttacatatatgtgttcttattgtttttaatatataaagtggacttgaatattgggctagcttagtataaaggaggttaaattagtttttaatgtttgattattataattttgaggatactgagttttacagtttggtatttttccttattagggtggcagacatgttgattatgtagctgatcagattgtgactaaacttgttgatgttgtgaagaagaagaacaagggtggtgttgcagtaaaagcacatcaggtatgtgcttttggcagttttctttttctaaagtcaaggaagaagagaaaggctataaataaagcatgagtacatttttagtggcttaatatcaacttctattgcaggtgaaaaatcacatgtggatttttgtaaatgccttaattgaaaacccaacctttgactctcagacaaaagaaaacatgactttacaacccaagagctttggatcaacatgccaattgagtgaaaaatttatcaaagctgtgagtacttagaggaaaataaaaatagaaacacctgactttattttccattgcacttcttagctctgcagaaacaatgattcttctcatagtgagcttctccaagtcttcccaatctgaaaaggaagtaaaaaagggctttactttaactgatttaccaaagacttaatgaccgtctatatttcagtatttcccaattacattttaccattaagcttagatcacttttgaattaatctagctgtttaacaaacaccctcacttaaatgcctaagacttgctttcagtcaacacatccaaaattgaatttgttacctccatactcactgatttgcccatacaagcagccccccactctccaacaaaaaaacaacttcctatcttagtaaaaagccccaaccaacctctaggttgtataaacaagaaagctgggagccttcctttatttcccctcctctctaatccggtcaataagaatcatctcttggatgctgcagtagcttctcaccattatctcttttttggtttactacaataggttcttaaccttcatactggttaagtcctttccttggaatgcttttgagtgacttttgtgttaaaacacccatttttatcttcactctcatttgaaatctttcaatgacttccactcagggaaagtccaaattccataatttggccaacaagaaagatctgctgtaatctaattacacctacttctccaactcatctcagtgccagtttttcgtatattgtcctgttgcttttaaattactgaaaagcacagtgctcttcccctcctcagagtttattcacatgctaatccctctgcatgaaatacatccttttcacctggctactttaggtcttgtcctttcctcaggaaagcctttactttctacccttccccccacctaagttggttccaatataatattgaacataccttattagcaaacttcttgcttatccataacacttataacactgtaacttattttatttctgtctttttttttaggcagagtctcgctctgtcgcccaggctggaatgcagtggcacgatctcggctcactgcaacctccgcctcctaggtttcagtgattctcttgcctcagcctcccgagtagctgagattacaggcatgcatcaccatgcctggctaatttttttgttgttgtattttcagtagagacagggtttcaccatgttggtcaggctggtctcaaactcctgacctcaaatgatccacccatctcggccttccaaagtgttgggattacagccatgagccactgtgcccagtcttttttttttttttttttgagacagagtcttgctgtgttgcccaggctacagtgcagtgacgtaatcctggctcattgcaatctctgcctcccaggttcaagcgattctccttgcctcctgagtagctgggattctccagcctcctgagtagcggggattacagacatgcaccatcacatccagctaatttttgtatttttaatagagatggggttttaccatgttggccaggctggtcttgaactcctggcctcacttgatccaccatcctcggcctcctaaagtgctgtgattacaggcgtaagccactgcacccagctatgtctgtcttctatgttgtgtcttaaacttgatgaggacaagtgtctcaatttgttttatttgtgtctaattttatttatttatttttaatttttttttttggagacagcttctcactgtttcccaggctggagtgccgtggcttgatctcagttcactgcaacccgcgcctcccaggttaaagcaattctcctgcctcagcctcccaagcagctaggattacagccatttcaccaccaccatgcctggctacccttttttttttttttttttttttttttgagacggagtttcacttttgtcacccaggctggagtgcaatggtgcgatcttggctcgctgcaacctctacctcctgggttcaagcgattctcctgcctcagcctcccgagtagctggaattacaggtgcccaccaccacgccagctaatttttgtatttttagtagagccggggtttcgccatgttggccaggccggtctcaaactcctgacctcaggtgttctgcccaccttggcctcctaaagtgctgggattataggcgtgagccaccgtgcctggtctaatttgttttaaccactatatctccaacaagtagctcagtgctagcacaatataattatatagtaaatatttattgaacgaatgaaccaaaaggagcagctccctcagtggtgataacctgacatgggaagatgtgccaccctctatccagaaattattgttctacatctttttaatttttgaatcatttttatttgtattaaggctcatttgtattctagatttctgatagatcccttcttccctaatatgatccctaatatgaatcttctcgttttcaggccattggctgtggtattgtagaaagcatactaaactgggtgaagtttaaggcccaagtccagttaaacaagaagtgttcagctgtaaaacataatagaatcaagggaattcccaaactcgatgatgccaatgatgcaggtatatatttaataatgtttccaaacttttaagtcttatagttgttattttattcattaatggcataccacggatatttatttttcccttgacagaataactatattcaacagaataacttgttaaaaatcggcccgtttcctattatggaagatttaggtcatttccatgttataaataatattgaggtgattattttggagtataaaacaagaatgtttatattatgatctattacctaacaaataattttgctcattatatagtaaattgtgttttatcacaaggctataaacagcatgttcaagttagtatatttgaggttgaactaaatgtgctaatattaatatgtatatttttattttagggggccgaaactccactgagtgtacgcttatcctgactgagggagattcagccaaaactttggctgtttcaggccttggtgtggttgggagagacaaatatggggttttccctcttagaggaaaaatactcaatgttcgagaagcttctcataagcaggtagaatataagacgatcttcagaatctaaatctaatttataatacaagactttatgcttatatttaattccctcattaggcattttaaaatatattttagacaatttgtgcttattttgagaaattaggtacattgtagcctattttaacagacctttctgatgtagtaaattataagctaatagctcaaaatactggagctcaagaaaatccaagcaacatatactgttaaatttctttgttcttttcaaatttataaacgatgctttttttggtatatgtccatttcagatcatggaaaatgctgagattaacaatatcatcaagattgtgggtcttcagtacaagaaaaactatgaagatgaagattcattgaagacgcttcgttatgggaagataatgattatgacagatcaggtcagatttgttattaaatttttagattgttcaactaaattaagcatgtcttaatttaatttcattgttttttgccatgaaaataaattagttaaataggagctttattcatcatctctaatcaacatctaatcagatatgcttatatcatatgtatgttgcaaatacaggttaagtgagtctggatttgaacagaccttttttgattcccatagaaaatttgacaaattgccagtaggtcagtcataatatttttttatttctaaacaattctttgtttgtttgagatggagtttcgcccttgtcgcccaggctggagtgcaatggtgcaatcttggctcactgcaacctccgcctcatgggttcaagcgattctcctgcctcagcctcccgagtagctgggattgcaggcggatgccaccacacccaactaatttttgtatttttagtggagacagggtttcaccatgttggccaggctggtctcgaacgcctgacctcaggcgatccgcctgcctcggcctcccaaagttctgggattacagatgttagctaccacgcccagcctaacagttcttttgaactttggctttcaaatctttctaggaccaagatggttcccacatcaaaggcttgctgattaattttatccatcacaactggccctctcttctgcgacatcgttttctggaggaatttatcactcccattgtaaaggtacgctaatttctaagtaccatcatggatattttaagaccctactcctcaaacctggatatacatataagccccgtcacatgtagtggtatacaggggaccccacagtgtacaaagccacagtcattgttttttatcacaggtatctaaaaacaagcaagaaatggcattttacagccttcctgaatttgaagagtggaagagttctactccaaatcataaaaaatggaaagtcaaatattacaaaggtttgtaatgaaacccatatagaacttctcattttattatacaccctgtacaagactatatgaaggaacttggtatttttggttttataggtttgggcaccagcacatcaaaggaagctaaagaatactttgcagatatgaaaagacatcgtatccagttcaaatattctggtcctgaagatgatgctgctatcagcctggtaggtttgagttgtattttatatacattctaattttagaaatcactactttagccagttgaaacatttacatttttgtataagactccgtatcaaaaaaaaaagaatgttttatagaatagaatgtttccagtaagcatatcccagagaaggaatcagatattttagaaaattttgatttttggctgggcttggtggctcatgcctataattctaggactttgggaggccaaggcaggtggatcacctgagatcaagagttggagaccaacctgtccaacatggtgaaaccccctctctactaaaaatacgaaaattggccgagcgtagtggctcacgcctgtaatcccagcactttgggaggcagaggcgagcagatcacaaggtcaggagatcaagaccatcctggctaacaaggtgaaaccccatctctgctaaaaatacaaaaaattagccggccgtggtggcacgcagctatagtcccagctacttgggaagctgaggcaggagaatcgcttgaacccgagaggcagaggttgcagtgagccgagatcaggccactgctctccagcctgggcaacagagtgagactccatctcaaaaaaaaaggaaaaaggccaggcgtggtggctcacgcctgtaatgccagtactttggaaggctaagtcgggcagatcacaacgtcatgagatggagaccatcctggctaacacggtgaaaccccgtctctactaaaaataaaaaaaaataaaaaataaaaaattagccgggcttggtgacacgcgcctgtaatcccagctactccggaggctgaggcaggagaatggcgtgaacccaggaggcggagcttgcagtgagctgagatggtgccactgcactccagcctgggcgacagagcgagactctgtctcaaaaaaaaaaaaaaacaaaaaaggtcgggcgcagtggctcacaccagtaatcatagcactttgggaggccgaggcgggtggatcacctgagctcgggagttcaagaccagcctgaccaacatggtgaaaccccatctctactaaaaatacaaaatattagctgggtgtggtggcacgtgcctgtaatcccagctacacgggaggctgaggcaggagaatcacttgaacttgggaagtggaggttgtgggagccgaaatcgtgccatcacattccacaagagtgaaactccatttcaaaaaaaaaaaaatacaaaaattagctgggcgtggtggcgggcgcctttaatcccaggggctcaggaggctgaggcaggagaatcgcttgaacctaggacgcagaggttgcagtgagccaagatgacaccattgcactccaggctgggcaagagaacagtaactcccgtctcaaaaaaaaaaaaaaaggaaaacagtatttttgtttttctgttgttcgttctttgtagaaggatattttggaacctattagcctattagtaccagtgacatcttttcatcctttaatttataggcctttagcaaaaaacagatagatgatcgaaaggaatggttaactaatttcatggaggatagaagacaacgaaagttacttgggcttcctgaggtaaaagttttaaatatatgccacaaaatggattgttagactgaccttttggtattgatactatagcaaattaaacttactgaatagttattttagtaaaaaatttgatatgatagagttgtgcagcagttaaatttgctttatctttaaaacatataaaatatttctgtattattcaatattttttaaagacttaagaattacagaaataattcttagttctgaaaggtttctgtttgatttttgtgttgagcattgtttagaccggtagactcatgtaatactatttggtccttcaggattacttgtatggacaaactaccacatatctgacatataatgacttcatcaacaaggaacttatcttgttctcaaattctgataacgagagatctatcccttctatggtggatggtgagttccaatttgttagtctgttttcattgtaagatggaaatcaaattccaaaattggttaaattgaggatacttacgtttgctcttatttcattttaaaggtttgaaaccaggtcagagaaaggttttgtttacttgcttcaaacggaatgacaagcgagaagtaaaggttgcccaattagctggatcagtggctgaaatgtcttcttatcatcatggtgaggtaaacacacaatccatgtttccagaaagcattatatcagaaatccctgcagagtcattctcaaaacagatttggtgaaagtataagctcatatattcatagtaaagaacaagttggtaaaacctgtgaaagaataaaactgcttgccctttgatttctgaaagtaatattcctgcagggatattcataagtctgcaaaggtctacattaaaaaatgcttagtatgtttattataactttgtgatgtcaaaatttagacctccactgatttgaaaattgttataataaattgtgacacatgtatgcatggaatcagtgctgtaacttcagtaaaactgtgtgcactgacactgaggaatatccagggtctattctcaggtgaggaaaaaaagagacagaacaataactaaaaatgtctacaaggatatgttccaaattgttcactttagttacccctgagaactggaaagggcaactttcactttttatatgtgtctgaattgtttgactttacaccaatgttttgtgattactttttgcattaattatgtaaaacagttttaaagaactagattgcctacaggtttctagaattttaaatataatggttgtcttgttgctttcaatcaaaatatagtagggatacaataaattttcacccaacgaacatttgttgaatgcctaccatgtaccaggtatacaaaaataaataaaaatatgttccttatccccaagatataataacaatttagccagagggccaatacacacacagctatagaataaagtgatacatgctagaatagaaatgaacacagcattgtaacacaaaggaggaaatatttcattttgctgaggataaggagaagaactatgccttcaaagttgagtgggatttaatagaaattgttggacggcagccctaaacttggagccagaagttgtaggttcaagtactatttctgccatctattaatctgaggaaagtcactaaacaactagacctcacaaaatgttacaggattatagtgaggttaagtcataatgtatttgtattagtaaaagcctcagcttaatgaatctttttttccccacagatgtcactaatgatgaccattatcaatttggctcagaattttgtgggtagcaataatctaaacctcttgcagcccattggtcagtttggtaccaggctacatggtggcaaggattctgctagtccacgatacatctttacaatgctcaggtaggtatgctttcactttctagtgacttgccatttccagttttgaaacaatgtgagatttagttcaccaaaaatacaaaaactcagttaatcagaagatactggcatctagtggcagaggccaggggtgaggcttatacaggaaagcccccccataacaattatccagtgcaacatgtcaattatgctaaggttgagaaatcctgatgtagagcttgggactgagtaaaggagactttgattcttcctaaatatcacaattgaaattttactcctttacaagcctatggaaagtatattatgaatggtcaattttcagctgtcatcccagtcaccctttcagtggcattcaaacagttactaattgctccatccttcttgaaactctttatttggcctctatgactgtagactcttctggttctcttctaccttattagctttctctgctgggtccttctcaccttcccaacctctaaatactggcccattccagggctccttcatcagatctctttccaatgcctacactcaattcccaaatgatctcccattcatccgttctatacctttaaatctccagtcttggtctctcccctgaactcccaacttttatgtctgactgccctcttaacatttcccacttggatatctagttaagtatctcaaacataagtctaaaaccaaatccttagttttcctgtctcagtaaatggaatgccatgccatccttctaattgctcaggctaaaaaccttgtcacccttgactcctttgcttgatacttaacatccagttcctcagccaatcctataggatctgcattggaaaaatactaagccacccccatctcatctaaactattgtaacatatcatcttttcttaaagcagccaattattttttaaaaaataagtcactctgattcagaaccttcaactgctcagagtaaaatctaaagacctattttagctaaccagacattatgtgatccgtgtccccaccttccatgtcttttgattccttctcataccatctcctctccagccacactgtccttggcctatactcaaatataccgagcacatttcctcttcagggccttttacttactggttcctttgcctgcaacactctttctctagatatccacatggttcagtccctcagtttgttttttgttgtttttttttcttttgagatggagttttgctctttttgcccaggctggagtgcagtggcgcaatttcggctcattgcaacctctgcctcctgggttcaagtgattctcctgcctcagcctcccgtgttgctgggattacaggtacacgccaccatgctcagctaattttatatttttagtagagatgaggtttcaccatgttggccaggttggtcttgaactcctgacctcaggtgatctgcctccctcagcctcccaaagtgctgggattacaggcgcgagttactgcacctggcgccctcagtgtcttttctctgctcaaatgttttcctatcagaaacggcttaactttccttagcacctcttcagtcactttcccactttttcttctcttgtttatcccaccacctaacaaaatatgtgttaattgcaaacttctactggactgtgagctccttgaggattgagactttatgtccatgtgcgtgacacatagcaagtgctcaataaatattgttaaatgtatattagtcagcaagtaaaaccaagataattttcatgtataatacatgtcctttccataaaagttaagtgtgaaattcagtatttttgtaaaataacattctttgtttcttttctttagctctttggctcgattgttatttccaccaaaagatgatcacacgttgaagtttttatatgatgacaaccagcgtgttgagcctgaatggtacattcctattattcccatggtgctgataaatggtgctgaaggaatcggtactgggtggtcctgcaaaatccccaactttgatgtgcgtgaaattgtaaataacatcaggcgtttgatggatggagaagaacctttgccaatggtaagtattctgtgtgtgttaagagccttaacttttccttgggtttcagttgaatagattgtatgattaaagatgatgcaaacataaaatttaatttcatactttagatttattgttaaacgctgttgtgatttccgtctagtatcacatttagaaaacatactgtttttttaaatgccaatttagcttccaagttacaagaacttcaagggtactattgaagaactggctccaaatcaatatgtgattagtggtgaagtagctattcttaattctacaaccattgaaatctcagagcttcccgtcagaacatggacccaggtaaataattatggatttcttttttaggtttgtgatcaaaagaaataccttttgtactattgctgttgactattcagaagcctatattttagaaacttcaggattatttctttctttagacatacaaagaacaagttctagaacccatgttgaatggcaccgagaagacacctcctctcataacagactatagggaataccatacagataccactgtgaaatttgttgtgaagatgactgaagaaaaactggcagaggcagagagagttggactacacaaagtcttcaaactccaaactagtctcacatgcaactctatggtatgtatttgttttgtgagatgtacacgtttcaatttatattaaagattaatatcaattttaaaagtatatcttagtctacctttataaagtttagccaatttgtctattcttggctctaatcttgttttcctttttcaggtgctttttgaccacgtaggctgtttaaagaaatatgacacggtgttggatattctaagagacttttttgaactcagacttaaatattatggattaagaaaagaatggctcctaggaatgcttggtgctgaatctgctaaactgaataatcaggctcgctttatcttagagaaaatagatggcaaaataatcattggtatgttttgggaataataactgcttactaaaactctagttacgccaaactttctttaaacatttttggtgagagtaatggtggagaaagaggcaaaatccatttaattcacctggtcacttatacacgcatttctttaaaatatctgattaggtatttatagtttgaaagagatgatgtttccttgactgagcatcttgagaaatcaagatttagttgacaattagacatgaggagaatagagagctagaagaccttgcataaactgattgaccaagagaatagatacactaatcatgtctacaggaacagaaaataaaagagacagagaagagataataaatctgatggtaaaaaaaaaaaaaggcaggaagattacgaatggcttctactctctgggtgtggtggcgcatgcctgtaatctcagcacttgagctggggaggtcaaggctgcagtgagcctaggtagtgccactgcactccagcctggacacaagagtgagagagaccctgtctccaaaaaaaaatgatttgatcatatatgatttgactgcccccttgtggtaatttacatttgtcaatggtttagggagacttgcctgtataccgggatatacaaatttatgcaagcacgaagacagtttaatttccagttttaaacttttgacttgtgtaaaaccttatccccttcctaaaatattactcaaaatcattatctctgtttacttttttaaaaatagaaaataagcctaagaaagaattaattaaagttctgattcagaggggatatgattcggatcctgtgaaggcctggaaagaagcccagcaaaaggtaatcttgggtggcacttttctatattgttaaagactatgtattgtacaatgtttatgtttcacattttctaataaagctagattttaattaatcctttaggttccagatgaagaagaaaatgaagagagtgacaacgaaaaggaaactgaaaagagtgactccgtaacagattctggaccaaccttcaactatcttcttgatatgcccctttggtatttaaccaaggaaaagaaagatgaactctgcaggctaagaaatgaaaaagtgagttgatagtaggatggtacatgctgcttagttttgttctattctaatggtaaaagtaaaattgtgtgttgcatgaaaatgctgcttgtatacatatattaactcaattttgtaattatcttatgaaggaacaagagctggacacattaaaaagaaagagtccatcagatttgtggaaagaagacttggctacatttattgaagaattggaggtatgtagtttataatgcccatgttagaatttttattaatgaaataatatattccagcagtatacctttgtcaagatagttcacaattggcaataaaaagaaaataagaggcataaggataaattccaaatagcgtataaaagaacagattattggccaggtgcagtggctcacgctggtaatcccagcattttggaaggctgagttgggtggatcacttgaggtcaggggttcaagaccagcctggccaacatggtaaaaccccatttctactaaaaacacaaaaattagccaggcatgatggtgcataccagttactcaggaggctgaggcaggagaattgcttgaaccgagaggcagaggttgcagtgaaccgagatcacaccactgcactccagcctaggcaacacagcaagactccgtctcaaaaaaaaaaaaaaaaaaaaagggacagattattaaatactacttagaatacaaggccgggtgcggtggctcacacctacaatcccagcactttgggaggccgaagcaggcagatcacctgaggttgggagttcgagaccagcctgaccaacatggagaaaccctgtctctactaaaattgcaaaattagccaggcgtagtggcacatgcctgtaatcccagctacttgggaggctgaggcaggagaatcacttgaactcgggaggcagatgttgctgtgagctaagatcacaccattgcactccagcctgggcaacaagagtggaactccgtctcaaaaaaaaaaaaaaaaatactatttagaatacagtaaatgataccaggagactgcccagacattcagacatttctggacaaaaaaagaaaagagcaggagttgatttttgataaagggaagaacatattaggactgagaagataaaaagagcctaaatgtggaagaagaccaccaacccagtcccccaagtcaacttaaaaggacaaagccacagggcatggtggctcatgcctgtaatcccagcactctgggaggctgaggtgggcagatcacttgaggtcaggagttcgagaccagcctggacaacatggtgaaaccctgtctctactaaaaatacaaaaattagttgggtgtggtggcatccgcctgtaatcccagctactcaggaggttgaggcaagaaaattgcttgaacccagaaggcagaagttgcagtgagccgagattgcgccactgtactccagcctgggtgacagagcaagactccatctaaaaaaaaaaaaaaaaaaaggagaaagcagggctatccagatggtttcagaaggttatttgttacttaaatcttcctaataacatttgtttctcaacattattttaacataagaaaaaaggccagcactgtggctcacacctgtaatcccaatgtgctttgggaggctgaggcaggaggatcacttgagattaggagttcaaaaccagcttgggtaacgtagcaagacctcatctctacaaaatgttttttttaaatagccaggcatggtggtgtgggcctatagtctcagctactccggaggccaaggtgggaggatcacttgagaccaggagttcaaggctgcagtgaactatgatcatgtaccattgtactcagcctgcgtgtctcttaaaaaaaaaaaaacaaaaaaaaaaccaccaaaagctttatttcttaaatttaacatgcaaatttgtattactgcacttcagtatagatgcctagctcattgtaatcagttggttggataagtgtgcatcacttcctttcatattttgtatgtgagtttacaaatatatatgtatatacatgttttatttttagtttctcccttgttttcctcaaaaaggttaagtggtagaatcaggaataaaacccagtccttctgtctccacaccttccttttcaaggtgtgttttaattacatcttcactattgttgatagtatactaggttatggagcaatttatcactagtttttaagatgtaacaaaatgaaatcagtttaactgttgggtgtttagatattgtaatgtttttgttaacatttaaacttgctttcttatatcccttctgtaggctgttgaagccaaggaaaaacaagatgaacaagtcggacttcctgggaaaggggggaaggccaaggggaaaaaaacacaaatggctgaagttttgccttctccgcgtggtcaaagagtcattccacgaataaccatagaaatgaaagcagaggcagaaaagaaaaataaaaagaaaattaaggtaatactcttgtggtggctcacacctataatcccagcactttgggggccaaggtgggtggatcacttgaggtcagtggttcaagaccagcctcccaacatggttgaaaccctgtctctactaaaaattaaaaaaattaggcgggcatggtggcaggcgcctgtaatcccagctactcgggagactgaggcagaagaatcgcttgaacccgggaggcagaggttgcagtgagccaaggtcacaccactgcactccagcctggccaacagagcaaaactccatctcaaaaaaaaaaaagaggtaatactcttaaaaatattatgcataaaatattgatgcatatgtccacttaaaattgaaaaaaaaatgtataaaatttttttgttgttgttgttgagatggagtctcgctgtgttacccaggctggagtgcaatggcaggatctcagctcacagcaacctccgcctcccgtattcaagcgattctcgtgcctcagcctcccaagtagttgggggcacatcaccacacccagctaatttttgtatttttagtagagatggggtttcaccatcttggccaggctggtctcgaactcctgacctcaagagatctgcccaccacggcctcccaaagtgctgggattacaggcgtgagccaccgctcccagccaaaaatgtataaaatctctaatcccatagttcagaatttattctatatccaaccctaactttgctatcttttagaatgaaaatactgaaggaagccctcaagaagatggtgtggaactagaaggcctaaaacaaagattagaaaagaaacagaaaagagaaccaggtattacaacatttttagaaaaatggtataaagtgaagattaaattgtgtttgttacctagtacattccacatagatgttcaaaacctttttgttgaattgagaaacgtgaaagaggactgtatctcaattctacttggacctctttttttttttttttttttttttgagacagtttcactcttgttgcccaggctggagtgcaatagcacaatctcggctcactgcaatcttcacctcctgggttcaagcgattctcctgcctcagcttcctgagtagctgggattataggtgcatgccaccacgcctggctgctttttgtatttttagtagagacggggtttcaccatgttagtcaggctggtctcaaactcctgaccttgtgatttgcccgccttggcctcccaaagtgctgggattacaggcgtgagccactgcccctggcctggacctctttaatatcgtccttgggatttgattcattatagaaaatattttgtaatgaagtagtttgttaatttgaattccttaattgggatctaagaaaatacccagtttattagatttcattttctagccacttacgaggttctagtatacaaagtttcttaagaaaagtgactacatattagtaatgtttgtagtatctctagagttcaacatcatgcttcggatataaataatgtccactcaatgaactaaatagaattaataattctgttaatcatttgataatgcctttactgtctactttttttgaaacatctttattgagatattcacatgccaaatttgcccatttaacgtataccatttggctgggcgccatggcttatgcctgtaatcccagcactttgggaggctgaggctggcggatcacttgaggtcaggagttcaagaccaccctggccaacatggtgaaaccccatctctactaaaaatacaaaaattagttgagtgtggtggtgcgtgcctgtagtcccagctactcgagaggctgaggcacgagtgtcgcttgaacccaggaggcagaggttgcagagagccaagactgtgccattgcactccagcctgggtgacagagcaagactccaactcaaaaataaaaataaagtatgccattcagtggttttagtatagacaaacagttttaatacaatttattgccccccaagaaaactgaactcattaacaatcatttgccatttgccaccaactcctctccactgggtgaccactaaaaaatctaggggcatttcctataaattaaatcatataatatgtggtcttttggagctgacttctttacttaacacattttcaagggtaacccatgtggcagcccatatattagtacttcattctttttttttttttttttttgaggcagagtttcgctctgtcgcccaggctggcatgcagtatgcagtagtagtgagatctcggctcactgcaacctccgcctgctaggttcaggcaattctcctgcctcagcctcccgaggagctgggattacaggcacgcaccaccatgcccaaataatttttgtatttttagtagaaacagggtttcaccatgttggccaggatggtctcaaactcctgacctcaagcgatctgcccacctcggcctcccattttgctaagcccgtgaaggtaaacctctgtgttttcttctaagcattttatagttttagctcttacatttaggtctgtgatccactttgaattagtttttgtatatggtgtaaggaaggtgtccaacctgattctttgcaggtagatatccaattatttcagtatcatatgttaaaaagacttctttctacaatgtttgtcttcacccttgtcaaaaatcaattgactataaatgttaaggtttatgtatggagtctcaattctgttccactgattgagctatatatgtccttgagtatctactttttttgagacagggtttcactcttgcccaggctggtcttgaaatcctggctcaagcaatccttctatctcagcctcccaagtagctgggactataggcactcgccaccatgactagctaatttctaaaagttttttgtattttgtagagacagtccttttttctttttttttctttttttgagacaagatttcactctgtctcccaggctggagtacagtggcatgatcacagcccactgcagcctcgacctcccaggctcaaacaatcctcctgcctctgcctccgcctccctagtggctggaaccacaggcgcactccaccatgcccattaatttctgtattttttgtagagatggggttttgccatgttgaccaggctggtctttagctcctgagctcaagcgatccgcctacctcaccctcccaaagtgctgagattacaggtgtgagccaccgcacccagccaagacagggtcttatatcttaccccagctgatctcaaacccctgggctcaagtaatcctcccgccccagcctccctaagtgctgggattacaggcatgagccactgtgcccggccacatttatttatgagattaagtgaccaagctatatgcaccacaaaaacagagaccatatctgtctcttttcacgattgtattctcagaactatcacaggagttaataaatttgaaggatggatggatgatagatggagaaaagtgggtctaatatataaaggtgattaatttctatctcccctctaggtacaaagacaaagaaacaaactacattggcatttaagccaatcaaaaaaggaaagaagagaaatccctggtctgattcagaatcagataggagcagtgacgaaagtaattttgatgtccctccacgagaaacagagccacggagagcagcaagtaaggaaaactaaagaacattatagataaactgtaagagtggaatggccaatatacttcagggtactttgccccagaaattactgtgtcttaatagagcagtaaattatgttaaagctttgaaacactgaagcatctttcaggagatttaaaaactaaaatattcatgaaatgttatgtcaacctatgaattgtttctcctactaccctctttgatttttttgtatatggtttttttactagcaaaaacaaaattcacaatggatttggattcagatgaagatttctcagattttgatgaaaaaactgatgatgaagattttgtcccatcagatgctagtccacctaagaccaaaacttccccaaagcaagtatcttatctaatatgggttttgtcatgattgtttctaatatattgttttttgcttgacacatttagaattggttgtcaggatttttttttttaattctagacttagtaacaaagaactgaaaccacagaaaagtgtcgtgtcaggtatgtatttaagtaaaagtagtgagacatctgccttactgggacactcagatccaactgggttctaatcctggctacattatccaagtacttcctttggaaataaatttgagttcttattaacattatgttgatactgttcactgggtttagtttcacatgtaaataggtactgcattcagtatactaaaagtaatcatagtgcttgccaggtactttgctaggtgctagagatacaaaaaataaaaactgttcttgctgataaaaaattcagaaattagaacaaagttttaagagtatgttttaaatattccagaaaagtcacatatttggtgtttaaataggaattcataccagggacaaagcagaaaatggattttcatgtgttgcttaaatgtactattttcctttccatttgaaataatagaccttgaagctgatgatgttaagggcagtgtaccactgtcttcaagccctcctgctacacatttcccagatgaaactgaaattacaaacccagttcctaaaaagaatgtgacagtgaagaagacagcagcaaaaagtaagcctaaatctttgagatgggttaatgttgcaattacctaactggtttccacgtgtctatttcaatttttttattgccaaaacttactattgatattacagattaaatgttttccaattggaagcaatttcttttcgatctttataatcaaaattagtagtcaaggctgttccaaaacagtaagttatctctattgattgttcagttacagatcaaactccttgttctactcttttccctccttctcactactgcacttgactagtcaaaaaacaaaacaaaagccaggcagagtggctcatgcctgtaatcccagcactttgggaggctaaaacaagaggattgcttgaggccaggagttcaaggctgcagtgagctatatgatcaagccactgcactccagcctgagtgagagagcaagaccctgtctccaaaaataaggtagtccaaatgtttaaaatcagtgagtttctctcagtaccaggcttcatctagttcatttctgtgacaaggataggatttataagcaattgcaatgtttaacgtaaaacgtattcttgaaattgaattaagtttaaggctgggtgcagtggctcatgcctgtaatcccagcactttgggaggccaaggtgggtggatcacctgaggtcaggagttcgagactagcctggccaacatagtgaaacctcatctctactaaaaacacaaaattagccaggtgtggtggcacatgcctgtagtcagctactcgggaggctgaaacaggagaatcacttgaacccaggaggcagaggctacagtgagccgagatcgtgccactgcactccagcctgggcgagatagagcaagatgctgtctcaaaaaaaaaaaaaaaaaaatagaatgtttaagaatactttgattctctgttttcacctctcttagattgtcttttcctatgttaaatatacagtcatcacattgctgaagaaagttcgcaatgagaacaattcatctaagagtggctgtgactaggtcaggcgcggttgctcatgcctgtaatcccagcactttgggaggccgaggcgagtggatcacctgaggtcaggagtttgagaccagcttgaccaacatggtggaatcccatctctactaaaaatacaaaaaattagccgggtgtggtggcacacgcctgtaatcccagctactcaggaggctgaggcaggagaatcgcttgaacccaggaggcagaggttgcagtgagccgagataacaccactgcactccagcctggacgatagagtgagaccccatctcaaaaaaagagcagctgtgacaaatgcctgtattgaattgcaggtcagtcttccacctccactaccggtgccaaaaaaagggctgccccaaaaggaactaaaagggatccagctttgaattctggtgtctctcaaaagcctgatcctgccaaaaccaagaatcgccgcaaaaggaagccatccacttctgatgattctgactctaattttgagaaaattgtttcgaaagcagtcacaagcaaggtgagtgttgatcctagtcagtccttttgctgtagatgttctgaaacacgtaactaagccattgctcttaaaaatttggcatatctttaagaaaattaactctcatattctgttagcttttactgtacatatttagttttaacaaagttaaatatgccacttatttgggcaatggaagagttggccttagatctgcttcttattacttggtagaaaatagaaaactccttgaatatagtgtcttgatacatttttttacattacaattatgttgtcagatttacaatgtgcaagttacctgggcttttctcttttagaaatccaagggggagagtgatgacttccatatggactttgactcagctgtggctcctcgggcaaaatctgtacgggcaaagaaacctataaagtacctggaagagtcagatgaagatgatctgttttaaaatgtgaggcgattattttaagtaattatcttaccaagcccaagactggttttaaagttacctgaagctcttaacttcctcccctctgaatttagtttggggaaggtgtttttagtacaagacatcaaagtgaagtaaagcccaagtgttctttagctttttataatactgtctaaatagtgaccatctcatgggcattgttttcttctctgctttgtctgtgttttgagtctgctttcttttgtctttaaaacctgatttttaagttcttctgaactgtagaaatagctatctgatcacttcagcgtaaagcagtgtgtttattaaccatccactaagctaaaactagagcagtttgatttaaaagtgtcactcttcctccttttctactttcagtagatatgagatagagcataattatctgttttatcttagttttatacataatttaccatcagatagaactttatggttctagtacagatactctactacactcagcctcttatgtgccaagtttttctttaagcaatgagaaattgctcatgttcttcatcttctcaaatcatcagaggccgaagaaaaacactttggctgtgtctataacttgacacagtcaatagaatgaagaaaattagagtagttatgtgattatttcagctcttgacctgtcccctctggctgcctctgagtctgaatctcccaaagagagaaaccaatttctaagaggactggattgcagaagactcggggacaacatttgatccaagatcttaaatgttatattgataaccatgctcagcaatgagctattagattcattttgggaaatctccataatttcaatttgtaaactttgttaagacctgtctacattgttatatgtgtgtgacttgagtaatgttatcaacgtttttgtaaatatttactatgtttttctattagctaaattccaacaattttgtactttaataaaatgttctaaacattgcaaccca

SEQ ID NO: 14 - DNA, 5753 bp, Homo sapiens
gattggctggtctgcttcgggcgggctaaaggaaggttcaagtggagctctcctaaccgacgcgcgtctgtggagaagcggcttggtcgggggtggtctcgtggggtcctgcctgtttagtcgctttcagggttcttgagccccttcacgaccgtcaccatggaagtgtcaccattgcagcctgtaaatgaaaatatgcaagtcaacaaaataaagaaaaatgaagatgctaagaaaagactgtctgttgaaagaatctatcaaaagaaaacacaattggaacatattttgctccgcccagacacctacattggttctgtggaattagtgacccagcaaatgtgggtttacgatgaagatgttggcattaactatagggaagtcacttttgttcctggtttgtacaaaatctttgatgagattctagttaatgctgcggacaacaaacaaagggacccaaaaatgtcttgtattagagtcacaattgatccggaaaacaatttaattagtatatggaataatggaaaaggtattcctgttgttgaacacaaagttgaaaagatgtatgtcccagctctcatatttggacagctcctaacttctagtaactatgatgatgatgaaaagaaagtgacaggtggtcgaaatggctatggagccaaattgtgtaacatattcagtaccaaatttactgtggaaacagccagtagagaatacaagaaaatgttcaaacagacatggatggataatatgggaagagctggtgagatggaactcaagcccttcaatggagaagattatacatgtatcacctttcagcctgatttgtctaagtttaaaatgcaaagcctggacaaagatattgttgcactaatggtcagaagagcatatgatattgctggatccaccaaagatgtcaaagtctttcttaatggaaataaactgccagtaaaaggatttcgtagttatgtggacatgtatttgaaggacaagttggatgaaactggtaactccttgaaagtaatacatgaacaagtaaaccacaggtgggaagtgtgtttaactatgagtgaaaaaggctttcagcaaattagctttgtcaacagcattgctacatccaagggtggcagacatgttgattatgtagctgatcagattgtgactaaacttgttgatgttgtgaagaagaagaacaagggtggtgttgcagtaaaagcacatcaggtgaaaaatcacatgtggatttttgtaaatgccttaattgaaaacccaacctttgactctcagacaaaagaaaacatgactttacaacccaagagctttggatcaacatgccaattgagtgaaaaatttatcaaagctgccattggctgtggtattgtagaaagcatactaaactgggtgaagtttaaggcccaagtccagttaaacaagaagtgttcagctgtaaaacataatagaatcaagggaattcccaaactcgatgatgccaatgatgcagggggccgaaactccactgagtgtacgcttatcctgactgagggagattcagccaaaactttggctgtttcaggccttggtgtggttgggagagacaaatatggggttttccctcttagaggaaaaatactcaatgttcgagaagcttctcataagcagatcatggaaaatgctgagattaacaatatcatcaagattgtgggtcttcagtacaagaaaaactatgaagatgaagattcattgaagacgcttcgttatgggaagataatgattatgacagatcaggaccaagatggttcccacatcaaaggcttgctgattaattttatccatcacaactggccctctcttctgcgacatcgttttctggaggaatttatcactcccattgtaaaggtatctaaaaacaagcaagaaatggcattttacagccttcctgaatttgaagagtggaagagttctactccaaatcataaaaaatggaaagtcaaatattacaaaggtttgggcaccagcacatcaaaggaagctaaagaatactttgcagatatgaaaagacatcgtatccagttcaaatattctggtcctgaagatgatgctgctatcagcctggcctttagcaaaaaacagatagatgatcgaaaggaatggttaactaatttcatggaggatagaagacaacgaaagttacttgggcttcctgaggattacttgtatggacaaactaccacatatctgacatataatgacttcatcaacaaggaacttatcttgttctcaaattctgataacgagagatctatcccttctatggtggatggtttgaaaccaggtcagagaaaggttttgtttacttgcttcaaacggaatgacaagcgagaagtaaaggttgcccaattagctggatcagtggctgaaatgtcttcttatcatcatggtgagatgtcactaatgatgaccattatcaatttggctcagaattttgtgggtagcaataatctaaacctcttgcagcccattggtcagtttggtaccaggctacatggtggcaaggattctgctagtccacgatacatctttacaatgctcagctctttggctcgattgttatttccaccaaaagatgatcacacgttgaagtttttatatgatgacaaccagcgtgttgagcctgaatggtacattcctattattcccatggtgctgataaatggtgctgaaggaatcggtactgggtggtcctgcaaaatccccaactttgatgtgcgtgaaattgtaaataacatcaggcgtttgatggatggagaagaacctttgccaatgcttccaagttacaagaacttcaagggtactattgaagaactggctccaaatcaatatgtgattagtggtgaagtagctattcttaattctacaaccattgaaatctcagagcttcccgtcagaacatggacccagacatacaaagaacaagttctagaacccatgttgaatggcaccgagaagacacctcctctcataacagactatagggaataccatacagataccactgtgaaatttgttgtgaagatgactgaagaaaaactggcagaggcagagagagttggactacacaaagtcttcaaactccaaactagtctcacatgcaactctatggtgctttttgaccacgtaggctgtttaaagaaatatgacacggtgttggatattctaagagacttttttgaactcagacttaaatattatggattaagaaaagaatggctcctaggaatgcttggtgctgaatctgctaaactgaataatcaggctcgctttatcttagagaaaatagatggcaaaataatcattgaaaataagcctaagaaagaattaattaaagttctgattcagaggggatatgattcggatcctgtgaaggcctggaaagaagcccagcaaaaggttccagatgaagaagaaaatgaagagagtgacaacgaaaaggaaactgaaaagagtgactccgtaacagattctggaccaaccttcaactatcttcttgatatgcccctttggtatttaaccaaggaaaagaaagatgaactctgcaggctaagaaatgaaaaagaacaagagctggacacattaaaaagaaagagtccatcagatttgtggaaagaagacttggctacatttattgaagaattggaggctgttgaagccaaggaaaaacaagatgaacaagtcggacttcctgggaaaggggggaaggccaaggggaaaaaaacacaaatggctgaagttttgccttctccgcgtggtcaaagagtcattccacgaataaccatagaaatgaaagcagaggcagaaaagaaaaataaaaagaaaattaagaatgaaaatactgaaggaagccctcaagaagatggtgtggaactagaaggcctaaaacaaagattagaaaagaaacagaaaagagaaccaggtacaaagacaaagaaacaaactacattggcatttaagccaatcaaaaaaggaaagaagagaaatccctggtctgattcagaatcagataggagcagtgacgaaagtaattttgatgtccctccacgagaaacagagccacggagagcagcaacaaaaacaaaattcacaatggatttggattcagatgaagatttctcagattttgatgaaaaaactgatgatgaagattttgtcccatcagatgctagtccacctaagaccaaaacttccccaaaacttagtaacaaagaactgaaaccacagaaaagtgtcgtgtcagaccttgaagctgatgatgttaagggcagtgtaccactgtcttcaagccctcctgctacacatttcccagatgaaactgaaattacaaacccagttcctaaaaagaatgtgacagtgaagaagacagcagcaaaaagtcagtcttccacctccactaccggtgccaaaaaaagggctgccccaaaaggaactaaaagggatccagctttgaattctggtgtctctcaaaagcctgatcctgccaaaaccaagaatcgccgcaaaaggaagccatccacttctgatgattctgactctaattttgagaaaattgtttcgaaagcagtcacaagcaagaaatccaagggggagagtgatgacttccatatggactttgactcagctgtggctcctcgggcaaaatctgtacgggcaaagaaacctataaagtacctggaagagtcagatgaagatgatctgttttaaaatgtgaggcgattattttaagtaattatcttaccaagcccaagactggttttaaagttacctgaagctcttaacttcctcccctctgaatttagtttggggaaggtgtttttagtacaagacatcaaagtgaagtaaagcccaagtgttctttagctttttataatactgtctaaatagtgaccatctcatgggcattgttttcttctctgctttgtctgtgttttgagtctgctttcttttgtctttaaaacctgatttttaagttcttctgaactgtagaaatagctatctgatcacttcagcgtaaagcagtgtgtttattaaccatccactaagctaaaactagagcagtttgatttaaaagtgtcactcttcctccttttctactttcagtagatatgagatagagcataattatctgttttatcttagttttatacataatttaccatcagatagaactttatggttctagtacagatactctactacactcagcctcttatgtgccaagtttttctttaagcaatgagaaattgctcatgttcttcatcttctcaaatcatcagaggccgaagaaaaacactttggctgtgtctataacttgacacagtcaatagaatgaagaaaattagagtagttatgtgattatttcagctcttgacctgtcccctctggctgcctctgagtctgaatctcccaaagagagaaaccaatttctaagaggactggattgcagaagactcggggacaacatttgatccaagatcttaaatgttatattgataaccatgctcagcaatgagctattagattcattttgggaaatctccataatttcaatttgtaaactttgttaagacctgtctacattgttatatgtgtgtgacttgagtaatgttatcaacgtttttgtaaatatttactatgtttttctattagctaaattccaacaattttgtactttaataaaatgttctaaacattgcaaccca

SEQ ID NO: 15 - protein, 1531 aa, Homo sapiens
MEVSPLQPVNENMQVNKIKKNEDAKKRLSVERIYQKKTQLEHILLRPDTYIGSVELVTQQMWVYDEDVGINYREVTFVPGLYKIFDEILVNAADNKQRDPKMSCIRVTIDPENNLISIWNNGKGIPVVEHKVEKMYVPALIFGQLLTSSNYDDDEKKVTGGRNGYGAKLCNIFSTKFTVETASREYKKMFKQTWMDNMGRAGEMELKPFNGEDYTCITFQPDLSKFKMQSLDKDIVALMVRRAYDIAGSTKDVKVFLNGNKLPVKGFRSYVDMYLKDKLDETGNSLKVIHEQVNHRWEVCLTMSEKGFQQISFVNSIATSKGGRHVDYVADQIVTKLVDVVKKKNKGGVAVKAHQVKNHMWIFVNALIENPTFDSQTKENMTLQPKSFGSTCQLSEKFIKAAIGCGIVESILNWVKFKAQVQLNKKCSAVKHNRIKGIPKLDDANDAGGRNSTECTLILTEGDSAKTLAVSGLGVVGRDKYGVFPLRGKILNVREASHKQIMENAEINNIIKIVGLQYKKNYEDEDSLKTLRYGKIMIMTDQDQDGSHIKGLLINFIHHNWPSLLRHRFLEEFITPIVKVSKNKQEMAFYSLPEFEEWKSSTPNHKKWKVKYYKGLGTSTSKEAKEYFADMKRHRIQFKYSGPEDDAAISLAFSKKQIDDRKEWLTNFMEDRRQRKLLGLPEDYLYGQTTTYLTYNDFINKELILFSNSDNERSIPSMVDGLKPGQRKVLFTCFKRNDKREVKVAQLAGSVAEMSSYHHGEMSLMMTIINLAQNFVGSNNLNLLQPIGQFGTRLHGGKDSASPRYIFTMLSSLARLLFPPKDDHTLKFLYDDNQRVEPEWYIPIIPMVLINGAEGIGTGWSCKIPNFDVREIVNNIRRLMDGEEPLPMLPSYKNFKGTIEELAPNQYVISGEVAILNSTTIEISELPVRTWTQTYKEQVLEPMLNGTEKTPPLITDYREYHTDTTVKFVVKMTEEKLAEAERVGLHKVFKLQTSLTCNSMVLFDHVGCLKKYDTVLDILRDFFELRLKYYGLRKEWLLGMLGAESAKLNNQARFILEKIDGKIIIENKPKKELIKVLIQRGYDSDPVKAWKEAQQKVPDEEENEESDNEKETEKSDSVTDSGPTFNYLLDMPLWYLTKEKKDELCRLRNEKEQELDTLKRKSPSDLWKEDLATFIEELEAVEAKEKQDEQVGLPGKGGKAKGKKTQMAEVLPSPRGQRVIPRITIEMKAEAEKKNKKKIKNENTEGSPQEDGVELEGLKQRLEKKQKREPGTKTKKQTTLAFKPIKKGKKRNPWSDSESDRSSDESNFDVPPRETEPRRAATKTKFTMDLDSDEDFSDFDEKTDDEDFVPSDASPPKTKTSPKLSNKELKPQKSVVSDLEADDVKGSVPLSSSPPATHFPDETEITNPVPKKNVTVKKTAAKSQSSTSTTGAKKRAAPKGTKRDPALNSGVSQKPDPAKTKNRRKRKPSTSDDSDSNFEKIVSKAVTSKKSKGESDDFHMDFDSAVAPRAKSVRAKKPIKYLEESDEDDLF